(19) United States
(12) Patent Application Publication
(10) Pub. No.: US 2024/0197502 A1
(43) Pub. Date: Jun. 20, 2024

(54) STENT AND STENT DELIVERY SYSTEM

(71) Applicant: OLYMPUS CORPORATION, Tokyo (JP)

(72) Inventors: Kazuhiro KAN, Hachioji-shi, PA (US); Shun NOGUCHI, Machida-shi, PA (US); Kanta SASAKI, Hachioji-shi (JP); Ko TAKITA, Hachioji-shi (JP); Yuka IMAOKA, Hachioji-shi (JP)

(21) Appl. No.: 18/588,630

(22) Filed: Feb. 27, 2024

Related U.S. Application Data
(63) Continuation of application No. PCT/JP2021/032009, filed on Aug. 31, 2021.

(51) Int. Cl. A61F 2/90; A61F 2/88

(52) U.S. Cl. CPC A61F 2/90; A61F 2/88

(57) ABSTRACT

A stent formed by weaving wires includes: a plurality of straight-line crossing portions, which are formed by crossing at least two straight-line portions of the wires and are arranged adjacent to each other in a circumferential direction of the stent; and a plurality of interlocking portions configured by intersecting a peak-shaped bent portion, in which the wire is bent in a first direction side which is one side of a longitudinal axis direction of the stent and becomes convex, and a valley-shaped bent portion, in which the wire is bent in a second direction side which is the other side of the longitudinal axis direction and becomes convex, and arranged so as to be adjacent to each other in the circumferential direction of the stent, wherein the interlocking portions and the straight-line crossing portions are arranged alternately in the longitudinal axis direction.

### CROSS-REFERENCE TO RELATED APPLICATIONS

This application claims priority based on PCT/JP2021/032009 filed on Aug. 31, 2021 and PCT/JP2022/032380 filed on Aug. 29, 2022, the contents of which are hereby incorporated by reference.

### TECHNICAL FILED

The present invention relates to a stent and a stent delivery system.

### BACKGROUND

A procedure is known in which a stent is placed and expanded for stenosis or obstruction (hereinafter referred to as “stenosis, etc.”) that occurs in the digestive tract. A stent delivery system is used to place a stent in a stenosis or the like. A stent delivery system passes through a treatment instrument channel of an endoscope to deliver a stent to a stenosis or the like.

For example, the stent described in U.S. Pat. No. 6,974,472 (Patent Document 1) is constructed by winding a single wire around a pin attached to a jig and weaving it like a fence. The stent is formed with a portion (interlocking portion 60) where two curved shapes are interlocked with each other. Therefore, it has features such that it has a high shape-followability even when it is bent, and easily adapts to the topography of the lumen in which it is placed.

The stent described in Patent Document 1 is constructed by weaving a single wire. Therefore, the portions where the straight wires intersect each other (straight-line crossing portions 70) are positioned adjacent to the interlocking portions 60 in the circumferential direction of the stent. When the stent is bent, the interlocking portion 60 can bend to follow, but the straight-line crossing portion 70 generates a force (axial force) that opposes bending. Therefore, the shape followability of the stent as a whole is impaired. In particular, it is difficult to place a stent in a place where the shape of the lumen is greatly curved. Alternatively, even if the placement is successful, the lumen tends to conform to the shape of the stent, which may put a strain on the lumen, or may cause problems such as the stent moving from the placement position.

In light of the above circumstances, an object of the present invention is to provide a stent that is easy to bend and maintain a bent state, and a stent delivery system that includes the stent.

### SUMMARY

In order to solve the above problems, the present invention proposes the following means.

A stent according to a first aspect of the present invention is a stent formed by weaving wires, including: a plurality of straight-line crossing portions, which are formed by crossing at least two straight-line portions of the wires and are arranged adjacent to each other in a circumferential direction of the stent; and a plurality of interlocking portions configured by intersecting a peak-shaped bent portion, in which the wire is bent in a first direction side which is one side of a longitudinal axis direction of the stent and becomes convex, and a valley-shaped bent portion, in which the wire is bent in a second direction side which is the other side of the longitudinal axis direction and becomes convex, and arranged so as to be adjacent to each other in the circumferential direction of the stent, wherein the interlocking portions and the straight-line crossing portions are arranged alternately in the longitudinal axis direction.

A delivery system according to a second aspect of the present invention includes: an operation portion; an outer tubular member configured to extend distally from the operation portion; an inner tubular member configured to extend distally from the operation portion and located inside the outer tubular member; and the stent which is accommodated between the outer tubular member and the inner tubular member, wherein the operation portion is configured to place the stent by moving the outer tubular member or the inner tubular member in the longitudinal direction.

The stent of the present invention is easy to bend, and it is easy to maintain a bent state.

### BRIEF DESCRIPTION OF THE DRAWINGS

FIG. 1 is a diagram showing the overall configuration of an endoscope system with a stent according to a first embodiment of the present invention.

FIG. 2 is a diagram showing the overall configuration of the stent.

FIG. 3 is a development diagram in which the stent is deployed in the circumferential direction.

FIG. 4 is a development diagram of the stent containing a partial enlarged diagram.

FIG. 5 is a diagram showing a crossing between the first straight line and other wires.

FIG. 6 is a diagram showing an example of a transformation of a wire crossing portion.

FIG. 7 is a diagram showing a crossing between the first line and other wires in the transformation example.

FIG. 8 is a diagram showing other deformation examples of a wire crossing portion.

FIG. 9 is a diagram showing a crossing between the first line and other wires in the transformation example.

FIG. 10 is a development diagram in which a stent according to a second embodiment of the present invention is deployed in the circumferential direction.

FIG. 11 is a diagram showing an example of the arrangement of the placement of the first straight-line crossing portion and the second straight-line crossing portion.

FIG. 12 is a diagram showing other deformation examples of the placement of the first straight-line crossing portion and the second straight-line crossing portion.

FIG. 13 is a diagram showing other deformation examples of the placement of the first straight-line crossing portion and the second straight-line crossing portion.

FIG. 14 is a development diagram in which a stent according to a third embodiment of the present invention is deployed in the circumferential direction.

FIG. 15 is a diagram showing an example of the arrangement of the placement of the first straight-line crossing portion and the second straight-line crossing portion.

FIG. 16 is a diagram showing other deformation examples of the placement of the first straight-line crossing portion and the second straight-line crossing portion.

FIG. 17 is a development diagram in which a stent according to a fourth embodiment of the present invention is deployed in the circumferential direction.

FIG. 18 is a diagram showing an example of the arrangement of the placement of the first straight-line crossing portion and the second straight-line crossing portion.

FIG. 19 is a diagram showing other deformation examples of the placement of the first straight-line crossing portion and the second straight-line crossing portion.

FIG. 20 is a development diagram in which a stent according to a fifth embodiment of the present invention is deployed in the circumferential direction.

FIG. 21 is a diagram showing an example of a deformation of the placement of the first straight-line crossing portion and the second straight-line crossing portion.

FIG. 22 is a diagram showing other deformation examples of the placement of the first straight-line crossing portion and the second straight-line crossing portion.

FIG. 23 is a diagram showing other deformation examples of the placement of the first straight-line crossing portion and the second straight-line crossing portion.

FIG. 24 is a diagram showing other deformation examples of the placement of the first straight-line crossing portion and the second straight-line crossing portion.

FIG. 25 is a diagram showing a deformation example of the placement of a plurality of interlocking portions.

FIG. 26 is a development diagram in which a stent according to a sixth embodiment of the present invention is expanded in the circumferential direction.

FIG. 27 is an enlarged view of the area R3 shown in FIG. 26.

FIG. 28 is a development diagram in which a stent according to a seventh embodiment of the present invention is deployed in the circumferential direction.

FIG. 29 is an enlarged view of the area R4 shown in FIG. 28.

FIG. 30 is a development diagram of a deformation example of the stent.

FIG. 31 is a development diagram in which a stent according to an eighth embodiment of the present invention is expanded in the circumferential direction.

FIG. 32 is a development diagram in which a stent according to a ninth embodiment of the present invention is deployed in the circumferential direction.

FIG. 33 is a development diagram of the first wire that is woven in the stent manufacturing method, developed in the circumferential direction, according to a tenth embodiment of the present invention.

FIG. 34 is a development diagram in which the first wires and second wires are developed in the stent manufacturing method.

FIG. 35 is a diagram showing a stent according to an eleventh embodiment of the present invention.

FIG. 36 is a diagram showing a reduced diameter stent.

FIG. 37 is a development diagram in which the braided first wires are deployed in the circumferential direction in the manufacturing method of the stent.

FIG. 38 is a development diagram in which the braided first wires and second wires are deployed in the circumferential direction in the manufacturing method of the stent.

FIG. 39 is a diagram showing a deformation example of the stent.

FIG. 40 is a development diagram in which a stent according to a twelfth embodiment of the present invention is deployed in the circumferential direction.

FIG. 41 is a diagram showing a loop hitting part of the stent.

FIG. 42 is a diagram showing a loop hitting part of the stent.

FIG. 43 is a diagram showing a loop hitting part of the stent.

FIG. 44 is a diagram showing a loop hitting part of the stent.

FIG. 45 is a diagram showing a loop hitting part of the stent.

FIG. 46 is a diagram showing a loop hitting part of the stent.

FIG. 47 is a development diagram of a transformation example of the stent.

FIG. 48 is a development diagram of other deformation examples of the stent.

FIG. 49 is a development diagram of other deformation examples of the stent.

FIG. 50 is a development diagram of another deformation example of the stent.

FIG. 51 is a development diagram of other deformation examples of the stent.

FIG. 52 is a diagram showing how to knit an alternate example of the stent.

FIG. 53 is a diagram showing a knitting method of the end region of the alternative example of the stent.

FIG. 54 is a development diagram of a deformation example of a stent in the first embodiment.

FIG. 55 is a development diagram of a deformation example of a stent in the ninth embodiment.

FIG. 56 is a diagram showing a difference between a first arrangement embodiment and stent characteristics based on a second arrangement embodiment.

FIG. 57 shows an embodiment of a stent device delivery system with associated a stent device.

FIG. 58A shows a schematic view of the stent device with the stent body in a collapsed state.

FIG. 58B shows a schematic view of the stent device with the stent body in an expanded state.

FIG. 59 is a magnified view of an embodiment of a stent body and showing an aspect of the stent wires.

FIG. 60 is a magnified view of an embodiment of a stent body and showing an aspect of the stent wires.

FIG. 61A is a schematic view of a stent device and showing the arrangement of stent wires in a region of the stent body.

FIG. 61B is a schematic view of a stent device in magnified view and showing the arrangement of stent wires in a region of the stent body.

FIG. 61C is a schematic views of a stent device and showing the arrangement of stent wires in a region of the stent body.

FIG. 61D is a schematic views of a stent device in magnified view and showing the arrangement of stent wires in a region of the stent body.

FIG. 62A is a schematic view of a stent device placed within a patient's body under straight and bent configurations.

FIG. 62B is a schematic view of a stent device placed within a patient's body under straight and bent configurations.

FIG. 62C is a schematic view of a stent device placed within a patient's body under straight and bent configurations.

FIG. 63A is a schematic view showing an aspect of the stent wires having different interlocking features when the stent body is under different force conditions.

FIG. 63B is a schematic view showing an aspect of the stent wires having different interlocking features when the stent body is under different force conditions.

FIG. 64A is a table showing details of different types of loops and the relative ranking of each with respect to axial shortening and bending.

FIG. 64B is a schematic view showing an embodiment of the structures of the interlocking portions of the stent wire.

FIG. 64C is a schematic view showing an embodiment of the structures of the interlocking portions of the stent wire.

FIG. 65 is a table showing details of factors that may affect the functions of each loops.

FIG. 66A is a schematic view showing an embodiment of the structures of the interlocking portions of the stent wire.

FIG. 66B is a schematic view showing an embodiment of the structures of the interlocking portions of the stent wire.

FIG. 67A is a schematic view showing an embodiment of the structures of the interlocking portions of the stent wire.

FIG. 67B is a schematic view showing an embodiment of the structures of the interlocking portions of the stent wire.

FIG. 68 is a schematic view showing the location, distribution, and positional relationship of the interlocking portions in a stent device.

FIG. 69 is a schematic view showing the allocation of the interlocking portions in a stent device and showing details and arrangement of interlocking blocks in one embodiment.

FIG. 70A is a schematic view showing the allocation of the interlocking portions in a stent device and showing details and arrangement of interlocking blocks in other embodiment.

FIG. 70B is a schematic view showing the allocation of the interlocking portions in a stent device and showing details and arrangement of interlocking blocks in other embodiment.

FIG. 71A is a schematic view of the interlocking portions and a comparison picture of an example stent device.

FIG. 71B is a schematic view of the interlocking portions and a comparison picture of an example stent device.

FIG. 71C is a schematic view of the interlocking portions and a comparison picture of an example stent device.

FIG. 71D is a schematic view of the interlocking portions and a comparison picture of an example stent device.

FIG. 72A is a schematic view of the interlocking portions showing the effects of different interlocking portions to the flexibility of the stent device.

FIG. 72B is a schematic view of the interlocking portions showing the effects of different interlocking portions to the flexibility of the stent device.

FIG. 72C is a schematic view of the interlocking portions showing the effects of different interlocking portions to the flexibility of the stent device.

FIG. 73 is a schematic view of the interlocking portions showing the effects to the flexibility of the stent device for another interlocking portion.

FIG. 74 illustrates an example of a method for manufacturing of the stent device.

FIG. 75 illustrates an example of a method for manufacturing of the stent device.

FIG. 76 shows a related art stent device.

FIG. 77 shows a related art stent device.

### DETAILED DESCRIPTION

### First Embodiment

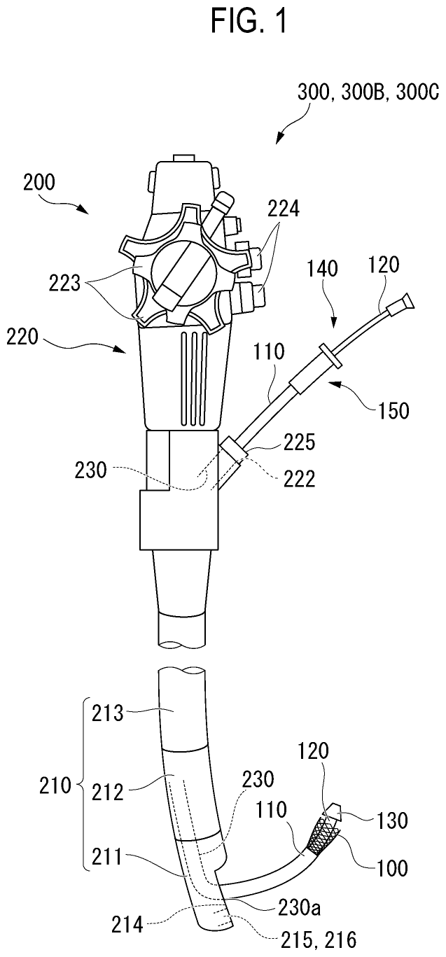

An endoscope system 300 having a stent 100 according to a first embodiment of the present invention will be described with reference to FIGS. 1 to 5. FIG. 1 is a diagram showing the overall configuration of the endoscope system 300.

### [Endoscope System 300]

The endoscope system 300 includes an endoscope 200 and a stent delivery system 150 inserted through a channel of the endoscope 200.

### [Endoscope 200]

The endoscope 200 is a known side-viewing flexible endoscope, and includes an elongated insertion portion 210 and an operation portion 220 provided at the proximal end of the insertion portion 210. Note that the endoscope 200 may be a direct-view flexible endoscope.

The insertion portion 210 includes a distal end rigid portion 211 provided at the distal end portion, a bendable bent portion 212 provided at the proximal end side of the distal end rigid portion 211, and a flexible tube portion 213 provided at the proximal end side of the bent portion 212. An imaging unit 216 having a light guide 215 and a CCD is provided on the side surface of the distal end rigid portion 211 in a state of being exposed to the outside.

The insertion portion 210 is formed with a treatment instrument channel 230 through which an endoscopic treatment instrument such as the stent delivery system 150 is inserted. A distal end portion 230a of the treatment instrument channel 230 is open on the side surface of the distal end rigid portion 211. A proximal end portion of the treatment instrument channel 230 extends to the operation portion 220.

A raising base 214 is provided on the distal end hard portion 211 of the treatment instrument channel 230. A proximal end portion of the raising base 214 is rotatably supported by the distal end rigid portion 211. An elevator operating wire (not shown) fixed to the distal end of the elevator 214 extends through the insertion portion 210 toward the proximal end.

The bent portion 212 is configured to be freely bendable in the vertical and horizontal directions. The distal end of the operation wire is fixed to the distal end side of the bent portion 212. The operation wire extends through the insertion portion 210 to the operation portion 220.

A knob 223 for operating the operation wire and a switch 224 for operating the imaging unit 216 and the like are provided on the proximal end side of the operation portion 220. The user can bend the bent portion 212 in a desired direction by operating the knob 223.

A forceps port 222 that communicates with the treatment instrument channel 230 is provided on the distal end side of the operation portion 220. A user can insert an endoscopic instrument such as the stent delivery system 150 through the forceps port 222. A forceps plug 225 is attached to the forceps port 222 to prevent leakage of bodily fluids.

### [Stent Delivery System 150]

The stent delivery system 150 is elongated as a whole and includes the stent 100, an outer tubular member 110, an inner tubular member 120, and an operation portion 140.

The outer tubular member 110 is made of resin or the like in a cylindrical shape and has flexibility. The outer tubular member 110 can be inserted through the treatment instrument channel 230 of the endoscope 200.

The inner tubular member 120 has an outer diameter smaller than the inner diameter of the outer tubular member 110 and can be passed through the inner space (lumen) of the outer tubular member 110. The inner tubular member 120 is made of resin or the like and has flexibility. A tip 130 having an outer diameter larger than that of the outer tubular member 110 is provided at the tip of the inner tubular member 120.

The stent 100 is housed at the distal end of the stent delivery system 150, as shown in FIG. 1. The stent 100 is accommodated in the gap between the inner tubular member 120 and the outer tubular member 110 in a state in which the inner tubular member 120 is passed through the inside thereof and the diameter thereof is reduced.

The operation portion 140 is connected to the proximal end sides of the outer tubular member 110 and the inner tubular member 120, and is configured to allow the outer tubular member 110 to move relative to the inner tubular member 120 in the longitudinal direction. By operating the operation portion, the operator moves the outer tubular member 110 with respect to the inner tubular member 120 to expose the accommodated stent 100, and as a result, the stent 100 can be placed. In addition, when the stent is exposed, the operator can move the outer tubular member 110 in the opposite direction relative to the inner tubular member 120, thereby allowing the stent 100 to be re-accommodated.

### [Stent 100]

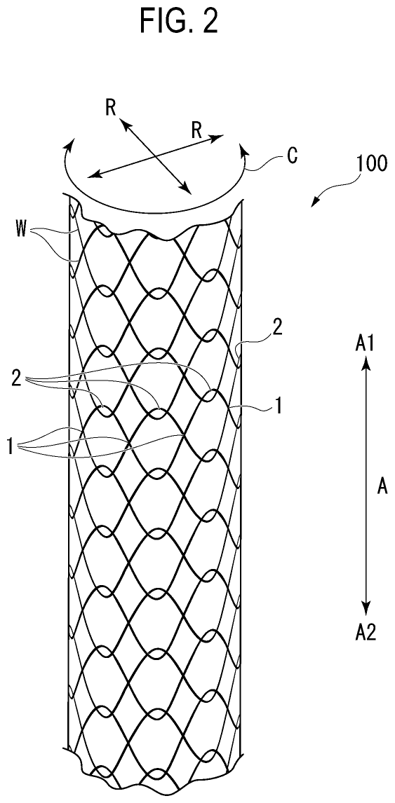

FIG. 2 is a diagram showing the overall configuration of the stent 100.

The stent 100 is formed by weaving wires and has a cylindrical shape. The stent 100 is indwelled in a digestive system body lumen such as the bile duct, esophagus, duodenum, small intestine, large intestine, etc., and is used mainly for the purpose of expanding and maintaining the lumen.

The stent 100 of this embodiment is not a so-called covered stent whose outer peripheral surface side is covered with a resin film or the like, but an uncovered stent that is not covered with a film or the like. However, the stent 100 can also be used as a covered stent by being covered with a resin film or the like.

In the following description, one of the longitudinal axis directions (axial directions) A of the stent 100 is called “first direction A1”, and the other of the longitudinal axis directions A of the stent 100 is called “second direction A2”.

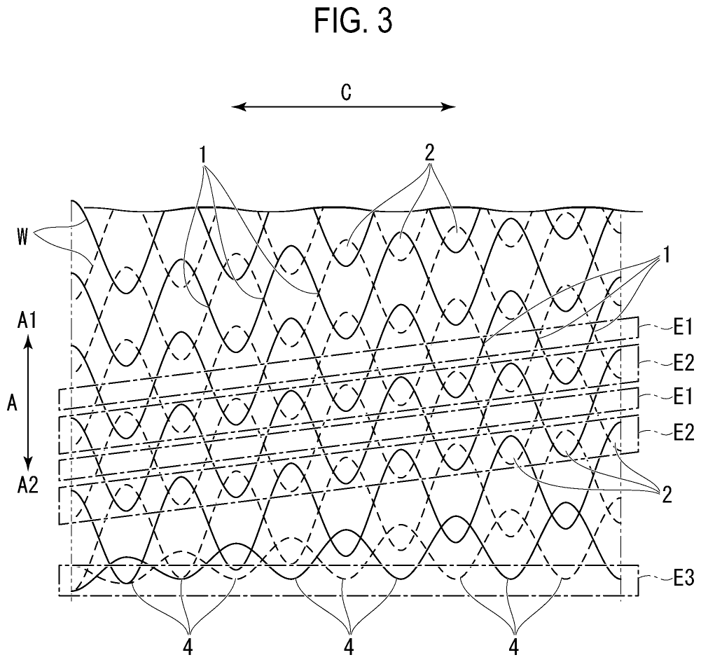

FIG. 3 is a developed view of the stent 100 deployed in the circumferential direction C.

The stent 100 is formed in the shape of a circular tube having meshes on its peripheral surface formed by wires W that extend obliquely in the circumferential direction C while repeatedly bending. The stent 100 has a plurality of straight-line crossing portions 1 and a plurality of interlocking portions 2.

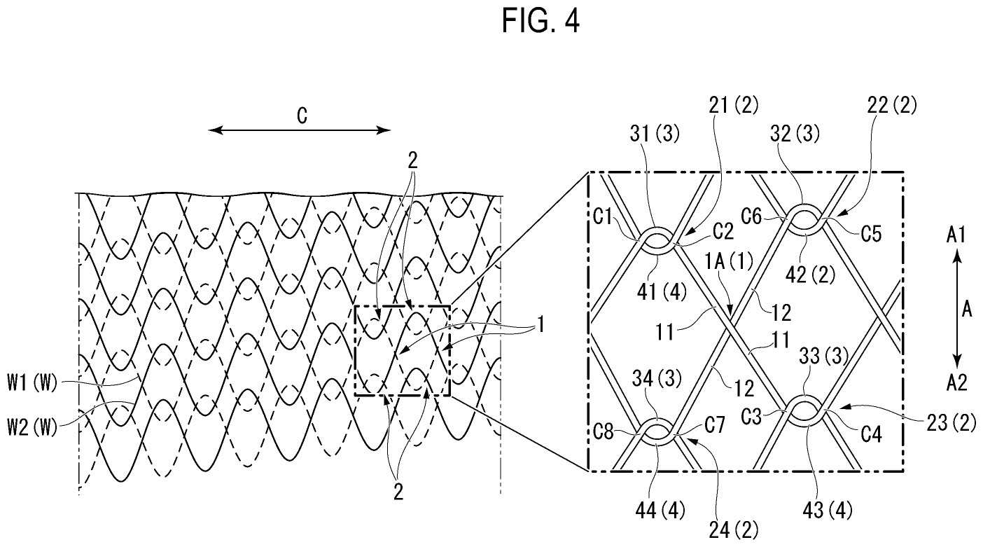

FIG. 4 is an exploded view of the stent 100 including a partially enlarged view.

The straight-line crossing portion 1 is formed by straight-line crossing of straight-line portions 10 of the wire W. Each straight-line portion 10 is a substantially straight-line portion of the wire W and includes a gently curved portion.

Each interlocking portion (engaging portion) 2 is formed by intersecting a peak-shaped bent portion 3 and a valley-shaped bent portion 4. The peak-shaped bent portion (peak) 3 is a convex portion in which the wire W extending obliquely in the circumferential direction C is folded back in the longitudinal axis direction A and convexes in the first direction A1. The valley-shaped bent portion (valley) 4 is a convex portion (concave in the first direction A1 side) in which the wire W extending in the circumferential direction is folded back in the longitudinal direction A and bent in the second direction A2 side (concave in the first direction A1 side). In the interlocking portion 2, the peak-shaped bent portion 3 and the valley-shaped bent portion 4 intersect each other in a hook shape, so that the peak-shaped bent portion 3 and the valley-shaped bent portion 4 are connected so as to be relatively movable although they cannot be separated.

As shown in FIG. 3, first regions E1 in which the plurality of straight-line crossing portions 1 are arranged and second regions E2 in which the plurality of interlocking portions 2 are arranged are alternately arranged in the longitudinal axis direction A. Each first region E1 is spirally arranged along the longitudinal axis direction A. In addition, each second region E2 is spirally arranged along the longitudinal axis direction A.

An end region E3 of the stent 100 on the second direction A2 side is arranged along the circumferential direction C without the valley-shaped bent portion 4 intersecting the peak-shaped bent portion 3. In addition, the end region (not shown) of the stent 100 on the first direction A1 side is arranged along the circumferential direction C without the peak-shaped bent portions 3 intersecting the valley-shaped bent portions 4.

The end portion of the valley-shaped bent portion 4 in the end region E3 on the second direction A2 side may be arranged spirally without intersecting the peak-shaped bent portion 3 as shown in FIG. 3, and may be aligned with respect to the longitudinal axis direction A. The same applies to the ends of the peak-shaped bent portions 3 on the first direction A1 side in the end regions (not shown) of the stent 100 on the first direction A1 side. For example, as in the end regions of the stent 100 shown in FIG. 34 and the end region of a stent 100K shown in FIG. 38, the positions of the ends with respect to the longitudinal axis direction A can be aligned by adjusting the length of the longitudinal axis direction A of the peak-shaped bent portion 3 and the valley-shaped bent portion 4 in the end region.

### [Central Straight-Line Crossing Portion 1A]

A first straight-line portion 11 and a second straight-line portion 12, which are the straight-line portions 10, intersect at the “central straight-line crossing portion 1A”, which is the straight-line crossing portion 1, as shown in FIG. 4. Specifically, when viewed from the radial direction R (see FIG. 2) of the stent 100, the first straight-line portion 11 and the second straight-line portion 12 intersect at the central straight-line crossing portion 1A. At the central straight-line crossing portion 1A, the first straight-line portion 11 passes outside the second straight-line portion 12 in the radial direction R.

### [First Interlocking Portion 21]

On the first direction A1 side of the first straight-line portion 11, a “first peak 31”, which is the peak-shaped bent portion 3, continues. The first peak 31 intersects with a first valley 41 that is the valley-shaped bent portion 4 to form the “first interlocking portion 2” that is the interlocking portion 2.

Specifically, when viewed from the radial direction R of the stent 100, the first peak 31 and the first valley 41 intersect at a first crossing portion C1 and a second crossing portion C2 closer to the central straight-line crossing portion 1A than the first crossing portion C1. At the first crossing portion C1, the first peak 31 passes through the outer side of the first valley 41 in the radial direction R. At the second crossing portion C2, the first peak 31 passes through the inner side of the first valley 41 in the radial direction R.

### [Second Interlocking Portion 22]

A “second peak 32”, which is the peak-shaped bent portion 3, is connected to the first direction A1 side of the second straight-line portion 12. The second peak 32 intersects with a second valley 42 that is the valley-shaped bent portion 4 to form the “second interlocking portion 22” that is the interlocking portion 2.

Specifically, when viewed from the radial direction R of the stent 100, the second peak 32 and the second valley 42 intersect at a fifth crossing portion C5 and a sixth crossing portion C6 closer to the central straight-line crossing portion 1A than the fifth crossing portion C5. At the fifth crossing portion C5, the second peak 32 passes inside the second valley 42 in the radial direction R. At the sixth crossing portion C6, the second peak 32 passes outside the second valley 42 in the radial direction R.

### [Third Interlocking Portion 23]

On the second direction A2 side of the first straight-line portion 11, a “third valley 43”, which is the valley-shaped bent portion 4, continues. The third valley 43 intersects with a third peak 33 that is the peak-shaped bent portion 3 to form the “third interlocking portion 23” that is the interlocking portion 2.

Specifically, when viewed from the radial direction R of the stent 100, the third peak 33 and the third valley 43 intersect at a fourth crossing portion C4 and a third crossing portion C3 closer to the central straight-line crossing portion 1A than the fourth crossing portion C4. The third valley 43 passes inside the third peak 33 in the radial direction R at the third crossing portion C3. The third valley 43 passes outside the third peak 33 in the radial direction R at the fourth crossing portion C4.

### [Fourth Interlocking Portion 24]

On the second direction A2 side of the second straight-line portion 12, a “fourth valley 44”, which is the valley-shaped bent portion 4, continues. The fourth valley 44 intersects with a fourth peak 34 that is the peak-shaped bent portion 3 to form the “fourth interlocking portion 24” that is the interlocking portion 2.

Specifically, when viewed from the radial direction R of the stent 100, the fourth peak 34 and the fourth valley 44 intersect at an eighth crossing portion C8 and a seventh crossing portion C7 closer to the central straight-line crossing portion 1A than the eighth crossing portion C8. The fourth valley 44 passes outside the fourth peak 34 in the radial direction R at the seventh crossing portion C7. The fourth valley 44 passes inside the fourth peak 34 in the radial direction R at the fourth crossing portion C4.

### [Arrangement of Straight-Line Crossing Portion 1 and Interlocking Portion 2]

The first interlocking portion 21 and the second interlocking portion 22 are arranged at different positions in the longitudinal axis direction A. Specifically, the second interlocking portion 22 is arranged on the first direction A1 side of the first interlocking portion 21 in the longitudinal axis direction A. Also, the first interlocking portion 21 is arranged in the longitudinal axis direction A between the second interlocking portion 22 and the central straight-line crossing portion 1A.

The third interlocking portion 23 and the fourth interlocking portion 24 are arranged at different positions in the longitudinal axis direction A. Specifically, the third interlocking portion 23 is arranged on the first direction A1 side of the fourth interlocking portion 24 in the longitudinal axis direction A. Further, the third interlocking portion 23 is arranged between the central straight-line crossing portion 1A and the fourth interlocking portion 24 in the longitudinal axis direction A.

The central straight-line crossing portion 1A is arranged between the first interlocking portion 21 and the second interlocking portion 22 in the circumferential direction C. Further, the central straight-line crossing portion 1A is arranged between the third interlocking portion 23 and the fourth interlocking portion 24 in the circumferential direction C.

### [Wire W]

The first peak 31, the first straight-line portion 11, and the third valley 43 are continuous parts of the wire W extending in a zigzag along the circumferential direction C, and are parts of the wire W indicated by broken lines in FIG. 4. The wire W indicated by the dashed line in FIG. 4 is also referred to as “first wire W1”.

The second peak 32, the second straight-line portion 12, and the fourth valley 44 are continuous parts of the wire W extending in a zigzag along the circumferential direction C, and are parts of the wire W indicated by solid lines in FIG. 4. The wire W indicated by the solid line in FIG. 4 is also referred to as “second wire W2”.

The first wire W1 and the second wire W2 may be one continuous wire, or may be different wires.

### [Crossing Portion of Wires W]

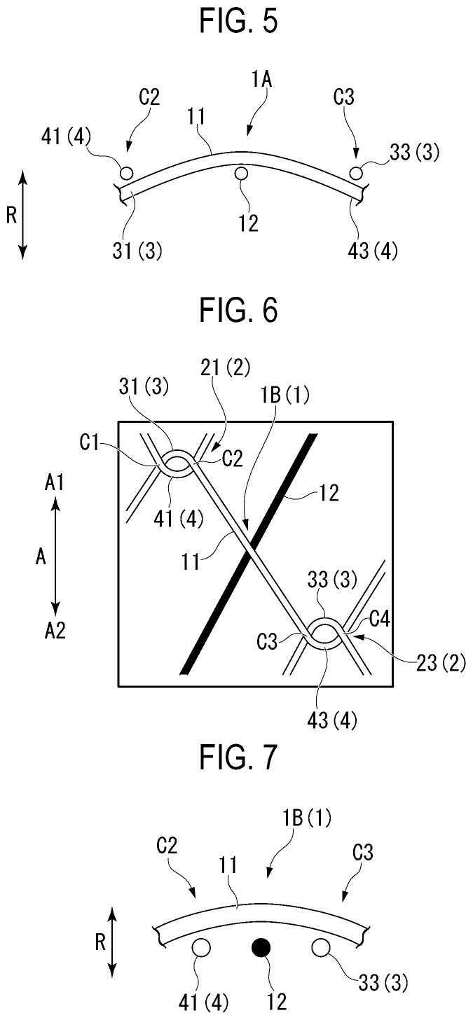

FIG. 5 is a diagram showing the crossing portion of the first straight-line portion 11 and another wire W.

In the first straight-line portion 11, the first peak 31 continuous in the first direction A1 passes inside the first valley 41 in the radial direction R at the second crossing portion C2. Further, in the first straight-line portion 11, the third valley 43 continuous in the second direction A2 passes inside the third peak 33 in the radial direction R at the third crossing portion C3. Therefore, as shown in FIG. 5, the first straight-line portion 11 sandwiched between the second crossing portion C2 and the third crossing portion C3 has a convex shape. As a result, the frictional force of the wires W intersecting at the second crossing portion C2, the third crossing portion C3 and the central straight-line crossing portion 1A increases, and the stent 100 tends to maintain its bent state.

### [Other Straight-Line Crossing Portions 1 and Interlocking Portions 2]

The central straight-line crossing portion 1A and the four interlocking portions 2 (the first interlocking portion 21, the second interlocking portion 22, the third interlocking portion 23 and the fourth interlocking portion 24) connected to the central straight-line crossing portion 1A have the above configuration. The other straight-line crossing portion 1 in the stent 100 and the four interlocking portions 2 connected to the straight-line crossing portion 1 have the same configuration.

### [Operation of Stent Delivery System 150]

A method for placing a stent using the endoscope system 300 including the stent delivery system 150 will be described by taking a procedure for placing the stent 100 in the bile duct as an example.

The operator inserts the insertion portion 210 of the endoscope 200 into the patient's body cavity through a natural opening such as the mouth. At that time, the operator bends the bent portion 212 by operating the knob 223 or the like as necessary.

The operator passes the guidewire through the treatment instrument channel 230 of the endoscope 200 and inserts the guidewire into the bile duct while observing with the endoscope 200. Subsequently, the operator operates the guidewire under X-ray fluoroscopy to break through the narrowed site in the bile duct, and moves the distal end of the guidewire to the liver side of the narrowed site (target position).

The operator inserts the proximal end of the guide wire protruding from the forceps plug 225 of the endoscope 200 into a through-hole of the tip 130 of the stent delivery system 150.

The operator advances the stent delivery system 150 along the guidewire by pushing the stent delivery system 150 while holding the guidewire. The distal end of stent delivery system 150 protrudes from the distal end of treatment instrument channel 230 of endoscope 200. After the distal end of the stent delivery system 150 breaks through the stenosis site (target position), the operator advances and retracts the stent delivery system 150 to determine the indwelling position of the stent 100. Note that the operator may insert the stent delivery system 150 into the treatment instrument channel 230 without using a guide wire.

After determining the target position of the stent 100, the operator retracts the outer tubular member 110 with respect to the inner tubular member 120. As a result, as shown in FIG. 1, the stent 100 is gradually exposed and expanded from the distal end side.

When the stent 100 is completely exposed, the stent 100 expands as a whole and the inner diameter of the stent 100 becomes larger than the outer diameter of the inner tubular member 120. Along with this, the locking between the stent 100 and the inner tubular member 120 is released.

After the locking between the stent 100 and the inner tubular member 120 is released, when the operator retracts the inner tubular member 120, the stent 100 remains at the indwelling position and the inner tubular member 120 is removed from the stent 100.

When the operator pulls out the stent delivery system 150 excluding the stent 100 from the body, the placement procedure of the stent 100 is completed.

The stent 100 of the present embodiment has a plurality of interlocking portions 2 and has high shape followability even when bent. Further, as shown in FIG. 5, since the first straight-line portion 11 sandwiched between the second crossing portion C2 and the third crossing portion C3 is convex, the frictional force of the wires W intersecting at the second crossing portion C2, the third crossing portion C3, and the central straight-line crossing portion 1A is increased, and the stent 100 can easily maintain its bent state. As a result, the stent 100 is easy to bend and easy to maintain in a bent state.

According to the stent 100 of the present embodiment, the second regions E2 are arranged in a spiral shape, so the stent 100 can be knitted without arranging the straight-line crossing portions 1 in the second regions E2. That is, in the stent 100, it is not necessary to arrange the straight-line crossing portion 1 at a position adjacent to the interlocking portion 2 in the circumferential direction C. Therefore, in the second region E2, the stent 100 has only the interlocking portions 2 having high conformability, and is easy to bend. The first regions E1 where the straight-line crossing portions 1 have a large frictional force (locking force) between the intersecting wires W are arranged alternately with the second regions E2 in the longitudinal axis direction A. Therefore, the stent 100 can preferably maintain its curved shape.

The first embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### (Modification 1-1)

The crossing mode of the wires W is not limited to the crossing mode shown in FIG. 5. FIG. 6 is a diagram showing a modification of the intersecting manner of the wires W. FIG. 7 is a diagram showing crossing portions of the first straight-line portion 11 and another wire W in the modification. In the modification shown in FIGS. 6 and 7, the first straight-line portion 11 has the first peak 31 continuous in the first direction A1 passing outside the first valley 41 in the radial direction R at the second crossing portion C2. Further, in the first straight-line portion 11, the third valley 43 continuous in the second direction A2 passes outside the third peak 33 in the radial direction R at the third crossing portion C3. Therefore, as shown in FIG. 7, the first straight-line portion 11 sandwiched between the second crossing portion C2 and the third crossing portion C3 has an arc shape. As a result, the frictional force of the wires W intersecting at the second crossing portion C2, the third crossing portion C3 and the central straight-line crossing portion 1B becomes smaller than in the above embodiment, and the stent 100 becomes more bendable.

### (Modification 1-2)

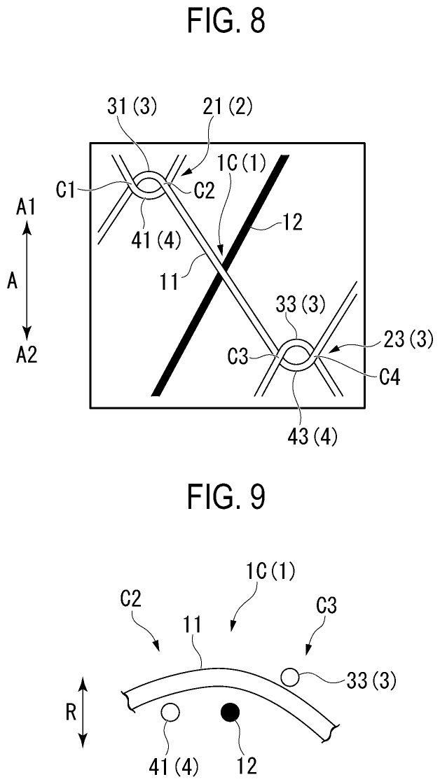

FIG. 8 is a diagram showing another modification of the wire W crossing mode. FIG. 9 is a diagram showing crossing portions of the first straight-line portion 11 and another wire W in another modification. In the modification shown in FIGS. 8 and 9, the first straight-line portion 11 has the first peak 31 continuous in the first direction A1 passing outside the first valley 41 in the radial direction R at the second crossing portion C2. Further, in the first straight-line portion 11, the third valley 43 continuous in the second direction A2 passes inside the third peak 33 in the radial direction R at the third crossing portion C3. As a result, the frictional force of the wires W intersecting at the second crossing portion C2, the third crossing portion C3, and the central straight-line crossing portion 1C becomes smaller than in the above embodiment, and larger than that in Modification 1-1.

### Second Embodiment

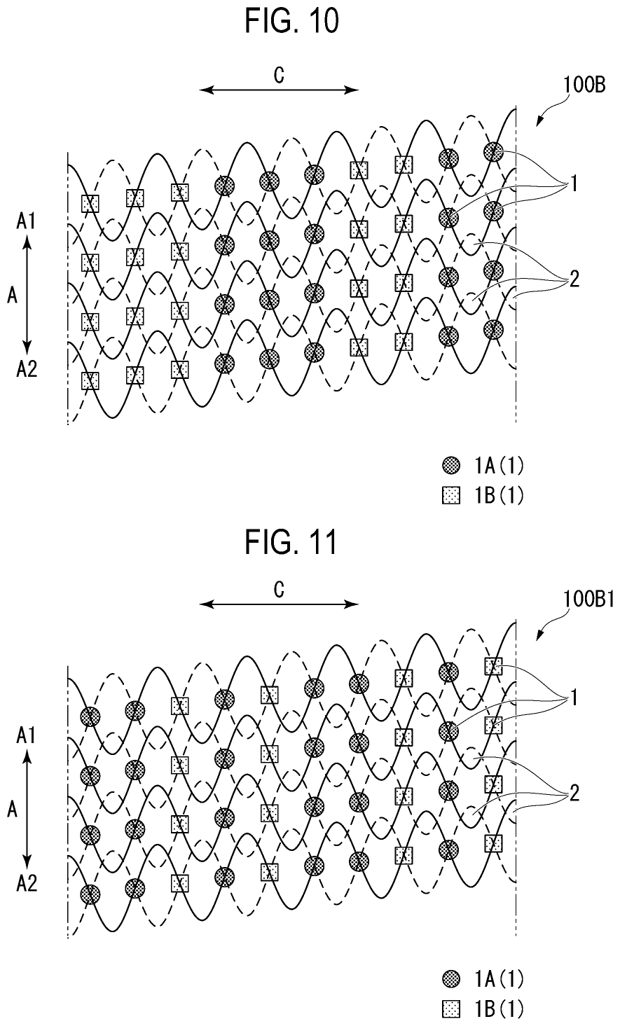

A second embodiment of the present invention will be described with reference to FIG. 10. In the following description, the same reference numerals are given to the same configurations as those already described, and redundant descriptions will be omitted. A stent 100B according to the second embodiment is housed in a stent delivery system 150, like the stent 100 according to the first embodiment.

In the stent 100 according to the first embodiment, the crossing portions of the wires W at the straight-line crossing portion 1 and the four interlocking portions 2 connected to the straight-line crossing portion 1 are the same at any location. In the stent 100B according to the second embodiment, the crossing manner of the wire W at the straight-line crossing portion 1 and the four interlocking portions 2 connected to the straight-line crossing portion 1 differs depending on the location.

### [First Straight-Line Crossing Portion 1A]

The central straight-line crossing portion 1A of the stent 100 according to the first embodiment will be referred to as “first straight-line crossing portion 1A” in the following description. Specifically, as shown in FIG. 5, in the first straight-line portion 11 forming the first straight-line crossing portion 1A, the first peak 31 extending in the first direction A1 passes inside the first valley 41 in the radial direction R at the second crossing portion C2. In addition, in the first straight-line portion 11 forming the first straight-line crossing portion 1A, the third valley 43 extending in the second direction A2 passes inside the third peak 33 in the radial direction R at the third crossing portion C3.

### [Second Straight-Line Crossing Portion 1B]

The central straight-line crossing portion 1B shown in Modification 1-1 of the first embodiment will be referred to as “second straight-line crossing portion 1B” in the following description. Specifically, as shown in FIG. 7, the first straight-line portion 11 forming the second straight-line crossing portion 1B has a first peak 31 extending in the first direction A1 and a first valley at the second crossing portion C2. 41 in the radial direction R. In addition, in the first straight-line portion 11 forming the second straight-line crossing portion 1B, the third valley 43 continuing on the second direction A2 side passes through the outside of the third peak 33 in the radial direction R at the third crossing portion C3.

The second straight-line crossing portion 1B may be a straight-line crossing portion 1 having a different wire W crossing mode such as the central straight-line crossing portion 1C shown in Modification 1-2.

FIG. 10 is a developed view of the stent 100B deployed in the circumferential direction C.

The stent 100B has a plurality of straight-line crossing portions 1 and a plurality of interlocking portions 2, like the stent 100 according to the first embodiment. The plurality of straight-line crossing portions 1 include a first straight-line crossing portion 1A and a second straight-line crossing portion 1B.

The first straight-line crossing portion 1A is arranged continuously in the longitudinal axis direction A. Two or three first straight-line crossing portions 1A are continuously arranged in the circumferential direction C. Moreover, the second straight-line crossing portion 1B is arranged continuously in the longitudinal axis direction A. Two or three second straight-line crossing portions 1B are continuously arranged in the circumferential direction C. Further, the first straight-line crossing portions 1A and the second straight-line crossing portions 1B are alternately arranged in the circumferential direction C.

In the stent 100B, the number of first straight-line crossing portions 1A and the number of second straight-line crossing portions 1B are substantially equal.

According to the stent 100B of this embodiment, the first straight-line crossing portion 1A where the frictional force of the crossing wires W is high is arranged continuously in the longitudinal axis direction A. Further, the second straight-line crossing portion 1B where the frictional force of the crossing wires W is low is arranged continuously in the longitudinal axis direction A. Further, the first straight-line crossing portions 1A and the second straight-line crossing portions 1B are alternately arranged in the circumferential direction C. As a result, the stent 100B can achieve both high shape followability and high shape retention.

The second embodiment of the present invention has been described above in detail with reference to the drawings, but the specific configuration is not limited to this embodiment, and design changes and the like are also included within the scope of the present invention. Also, the constituent elements shown in the above-described embodiment and modifications can be combined as appropriate.

### (Modification 2-1)

The arrangement of the first straight-line crossing portion 1A and the second straight-line crossing portion 1B is not limited to the arrangement shown in FIG. 10. FIG. 11 is a diagram showing a stent 100B1 that is a modification of the arrangement of the first straight-line crossing portion 1A and the second straight-line crossing portion 1B. In the stent 100B1, the number of the first straight-line crossing portions 1A is greater than the number of the second straight-line crossing portions 1B. Therefore, in the stent 100B1, the frictional force of the intersecting wires W is higher than in the stent 100B, and the stent 100B1 is less likely to be crushed in the longitudinal direction A.

### (Modification 2-2)

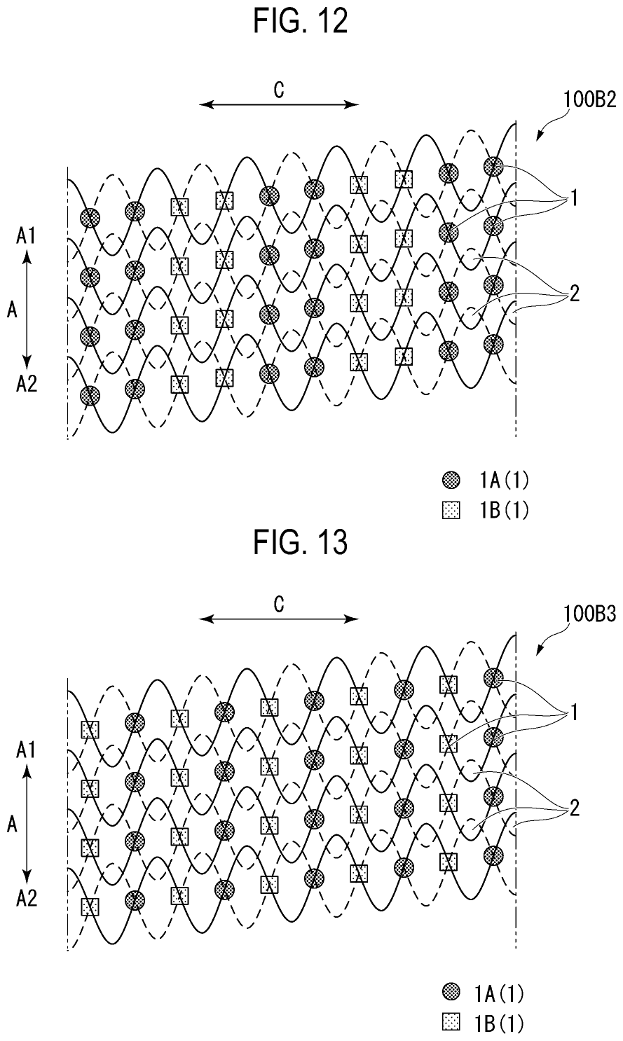

FIG. 12 is a diagram showing a stent 100B2 that is a modification of the arrangement of the first straight-line crossing portion 1A and the second straight-line crossing portion 1B. Two first straight-line crossing portions 1A are continuously arranged in the circumferential direction C. Further, two second straight-line crossing portions 1B are continuously arranged in the circumferential direction C.

### (Modification 2-3)

FIG. 13 is a diagram showing a stent 100B3 that is a modification of the arrangement of the first straight-line crossing portion 1A and the second straight-line crossing portion 1B. The first straight-line crossing portion 1A and the second straight-line crossing portion 1B are arranged alternately one by one in the circumferential direction C. The stent 100B3 has higher shape followability and shape retention than the stent 100B.

### Third Embodiment

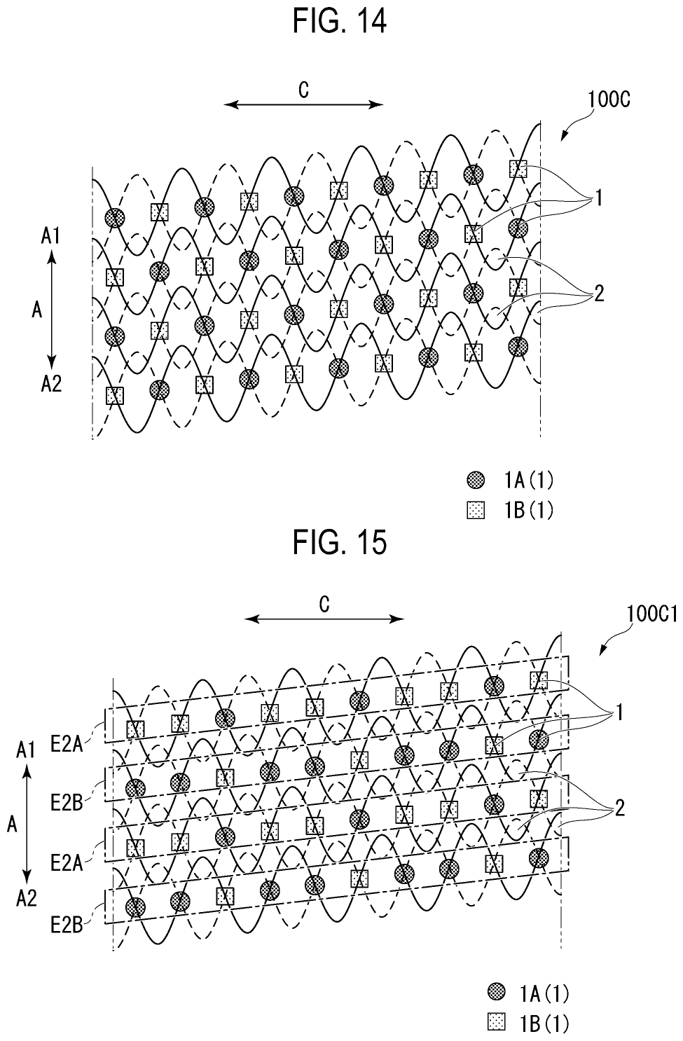

A third embodiment of the present invention will be described with reference to FIG. 14. In the following description, the same reference numerals are given to the same configurations as those already described, and redundant descriptions will be omitted. A stent 100C according to the third embodiment differs from the stent 100B according to the second embodiment only in the arrangement of the first straight-line crossing portion 1A and the second straight-line crossing portion 1B.

FIG. 14 is a developed view of the stent 100C deployed in the circumferential direction C.

The stent 100C has the plurality of straight-line crossing portions 1 and the plurality of interlocking portions 2, like the stent 100 according to the first embodiment. The plurality of straight-line crossing portions 1 include a first straight-line crossing portion 1A and a second straight-line crossing portion 1B.

The first straight-line crossing portion 1A and the second straight-line crossing portion 1B are alternately arranged one by one in the longitudinal axis direction A. Also, the first straight-line crossing portion 1A and the second straight-line crossing portion 1B are alternately arranged in the circumferential direction C one by one.

According to the stent 100C of this embodiment, the first straight-line crossing portion 1A where the frictional force of the crossing wires W is high and the second straight-line crossing portion 1B where the frictional force of the crossing wires W is low are alternately arranged both in the longitudinal direction A and in the circumferential direction C. Therefore, it is possible to achieve both high shape followability and high shape maintainability.

The third embodiment of the present invention has been described above in detail with reference to the drawings, but the specific configuration is not limited to this embodiment, and design changes and the like are included within the scope of the present invention. Also, the constituent elements shown in the above-described embodiment and modifications can be combined as appropriate.

### (Modification 3-1)

The arrangement of the first straight-line crossing portion 1A and the second straight-line crossing portion 1B is not limited to the arrangement shown in FIG. 14. FIG. 15 is a diagram showing a stent 100C1 that is a modification of the arrangement of the first straight-line crossing portion 1A and the second straight-line crossing portion 1B. In the stent 100C1, the first straight-line crossing portion 1A and the second straight-line crossing portion 1B are alternately arranged in the longitudinal axis direction A one by one.

The stent 100C1 includes a second region E2A in which one first straight-line crossing portion 1A and two continuous second straight-line crossing portions 1B are arranged in the circumferential direction C, and a second region E2B in which two consecutive first straight-line crossing portions 1A and one second straight-line crossing portion 1B are arranged in the circumferential direction C. The second region E2A and the second region E2B are alternately arranged in the longitudinal direction A one by one. The stent 100C1 has higher shape followability than the stent 100C.

### (Modification 3-2)

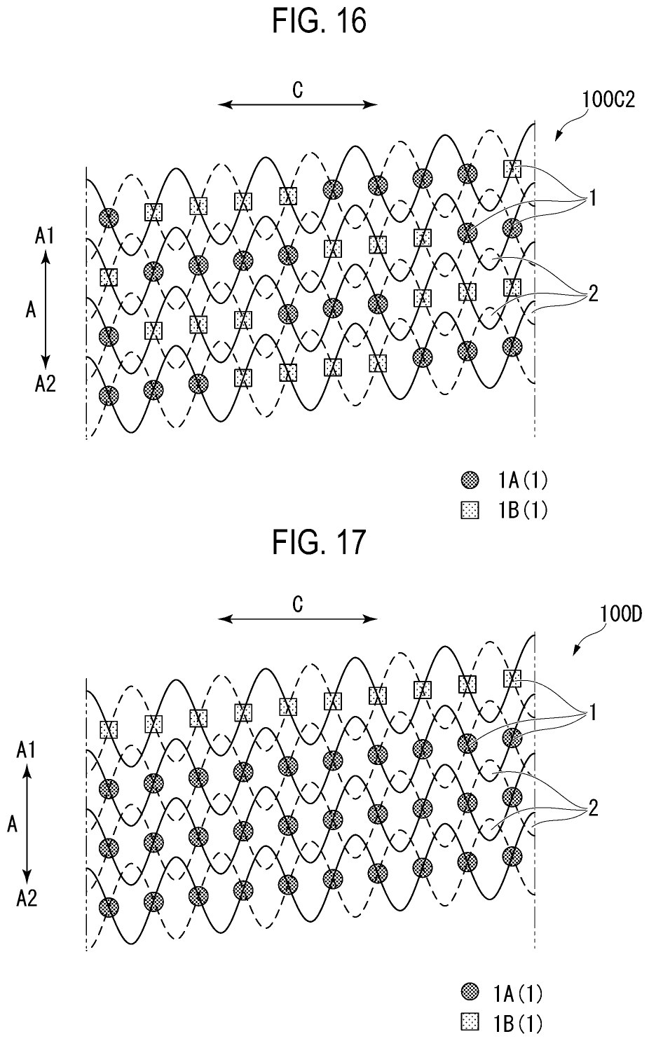

FIG. 16 is a diagram showing a stent 100C2 that is a modification of the arrangement of the first straight-line crossing portion 1A and the second straight-line crossing portion 1B. Three or four first straight-line crossing portions 1A are continuously arranged in the circumferential direction C. In addition, three or four second straight-line crossing portions 1B are continuously arranged in the circumferential direction C. The stent 100C2 can achieve both high shape followability and high shape retention.

### Fourth Embodiment

A fourth embodiment of the present invention will be described with reference to FIG. 17. In the following description, the same reference numerals are given to the same configurations as those already described, and redundant descriptions will be omitted. A stent 100D according to the fourth embodiment differs from the stent 100B according to the second embodiment only in the arrangement of the first straight-line crossing portion 1A and the second straight-line crossing portion 1B.

FIG. 17 is a developed view of the stent 100D deployed in the circumferential direction C.

The stent 100D has a plurality of straight-line crossing portions 1 and a plurality of interlocking portions 2, like the stent 100 according to the first embodiment. The plurality of straight-line crossing portions 1 include a first straight-line crossing portion 1A and a second straight-line crossing portion 1B.

The first straight-line crossing portions 1A are arranged continuously in the circumferential direction C. Moreover, the second straight-line crossing portion 1B is arranged continuously in the circumferential direction C. The first straight-line crossing portions 1A and the second straight-line crossing portions 1B are alternately arranged in the longitudinal axis direction A.

According to the stent 100D of the present embodiment, the first straight-line crossing portions 1A where the frictional force of the crossing wires W is high are arranged continuously in the circumferential direction C, so that it is easy to maintain a certain shape retention. As a result, the stent 100D can partially retain a portion having a very high shape followability while maintaining a certain shape retention property.

The fourth embodiment of the present invention has been described above in detail with reference to the drawings, but the specific configuration is not limited to this embodiment, and design changes and the like are included within the scope of the present invention. Also, the constituent elements shown in the above-described embodiment and modifications can be combined as appropriate.

### (Modification 4-1)

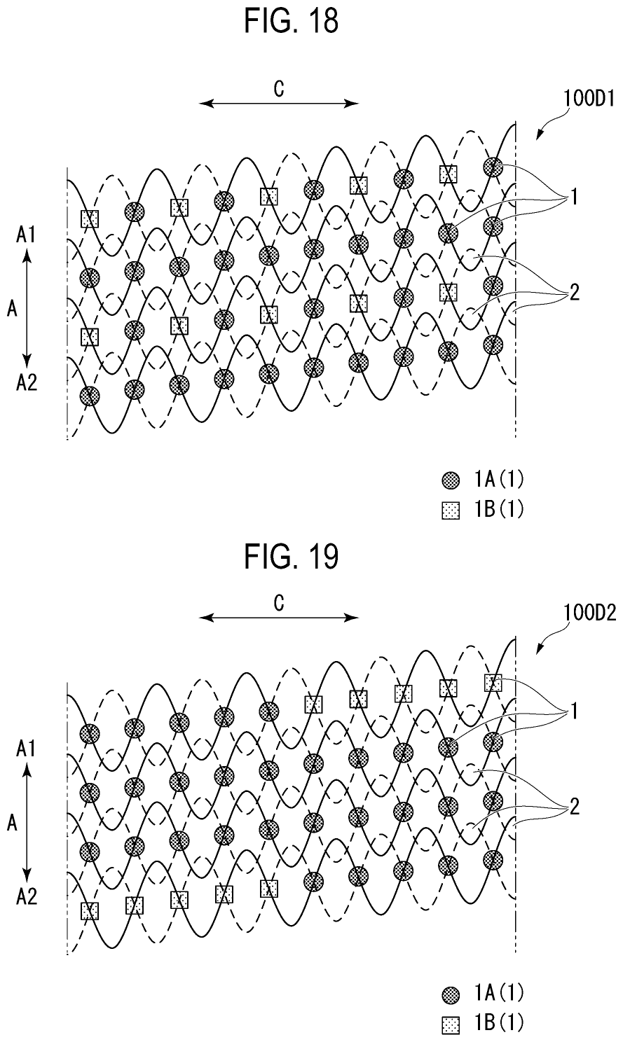

The arrangement of the first straight-line crossing portion 1A and the second straight-line crossing portion 1B is not limited to the arrangement shown in FIG. 17. FIG. 18 is a diagram showing a stent 100D1 that is a modification of the arrangement of the first straight-line crossing portion 1A and the second straight-line crossing portion 1B. In the stent 100D1, the first straight-line crossing portions 1A are alternately arranged in the circumferential direction C. In the stent 100D1, only the second straight-line crossing portion 1B is not arranged continuously in the circumferential direction C. The second straight-line crossing portions 1B and the first straight-line crossing portions 1A are alternately arranged in the circumferential direction C one by one. The stent 100D1 can partially retain a portion with extremely high shape followability while maintaining constant shape retention.

### (Modification 4-2)

FIG. 19 is a diagram showing a stent 100D2 that is a modification of the arrangement of the first straight-line crossing portion 1A and the second straight-line crossing portion 1B. In the stent 100D2, the first straight-line crossing portions 1A are arranged continuously in the circumferential direction C. In the stent 100D2, only the second straight-line crossing portion 1B is not continuously arranged in the circumferential direction C. The five continuous second straight-line crossing portions 1B are arranged adjacent to the five continuous first straight-line crossing portions 1A in the circumferential direction C. In the stent 100D2, the part where the second straight-line crossing portion 1B where the frictional force of the intersecting wires W is low is arranged intensively becomes a part that is easily curved. The stent 100D2 can partially enhance shape followability while maintaining high shape retention in a curved state.

### Fifth Embodiment

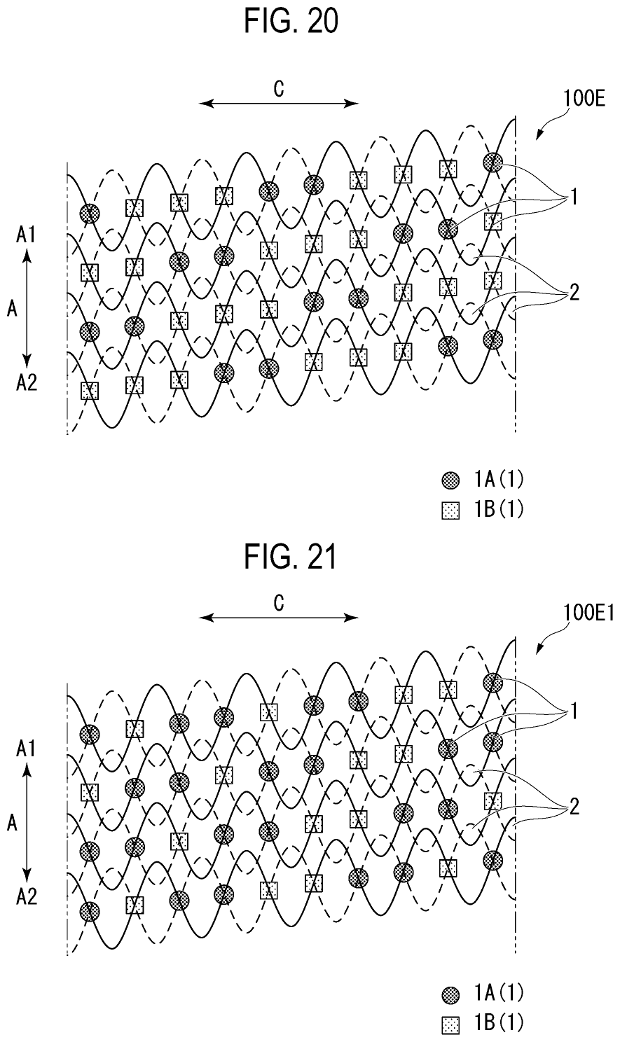

A fifth embodiment of the present invention will be described with reference to FIG. 20. In the following description, the same reference numerals are given to the same configurations as those already described, and redundant descriptions will be omitted. A stent 100E according to the fifth embodiment differs from the stent 100B according to the second embodiment only in the arrangement of the first straight-line crossing portion 1A and the second straight-line crossing portion 1B.

FIG. 20 is a developed view of the stent 100E deployed in the circumferential direction C.

The stent 100E has a plurality of straight-line crossing portions 1 and a plurality of interlocking portions 2, like the stent 100 according to the first embodiment. The plurality of straight-line crossing portions 1 include a first straight-line crossing portion 1A and a second straight-line crossing portion 1B.

The two first straight-line crossing portions 1A continuous in the circumferential direction C are arranged spirally along the longitudinal axis direction A. Further, three second straight-line crossing portions 1B continuous in the circumferential direction C are arranged spirally along the longitudinal axis direction A.

According to the stent 100E of this embodiment, since the first straight-line crossing portion 1A and the second straight-line crossing portion 1B are arranged in a spiral shape along the longitudinal axis direction A, it is possible to prevent twisting and improve shape followability.

The fifth embodiment of the present invention has been described above in detail with reference to the drawings, but the specific configuration is not limited to this embodiment, and design changes and the like are included within the scope of the present invention. Also, the constituent elements shown in the above-described embodiment and modifications can be combined as appropriate.

### (Modification 5-1)

The arrangement of the first straight-line crossing portion 1A and the second straight-line crossing portion 1B is not limited to the arrangement shown in FIG. 20. FIG. 21 is a diagram showing a stent 100E1 that is a modification of the arrangement of the first straight-line crossing portion 1A and the second straight-line crossing portion 1B. Two first straight-line crossing portions 1A continuous in the circumferential direction C are arranged spirally along the longitudinal axis direction A. Two second straight-line crossing portions 1B continuous in the circumferential direction C are arranged spirally along the longitudinal axis direction A.

### (Modification 5-2)

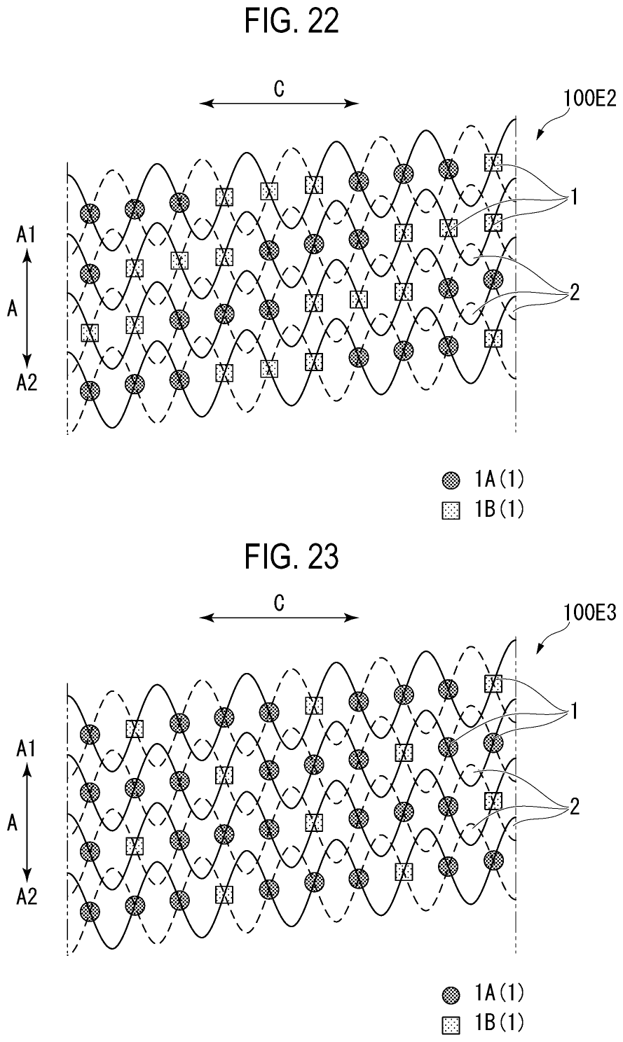

FIG. 22 is a diagram showing a stent 100E2 that is a modification of the arrangement of the first straight-line crossing portion 1A and the second straight-line crossing portion 1B. The three first straight-line crossing portions 1A continuous in the circumferential direction C are arranged spirally along the longitudinal axis direction A. Further, three second straight-line crossing portions 1B continuous in the circumferential direction C are arranged spirally along the longitudinal axis direction A.

### (Modification 5-3)

FIG. 23 is a diagram showing a stent 100E3 that is a modification of the arrangement of the first straight-line crossing portion 1A and the second straight-line crossing portion 1B. The three first straight-line crossing portions 1A continuous in the circumferential direction C are arranged spirally along the longitudinal axis direction A. Moreover, one second straight-line crossing portion 1B continuous in the circumferential direction C is arranged spirally along the longitudinal axis direction A.

### (Modification 5-4)

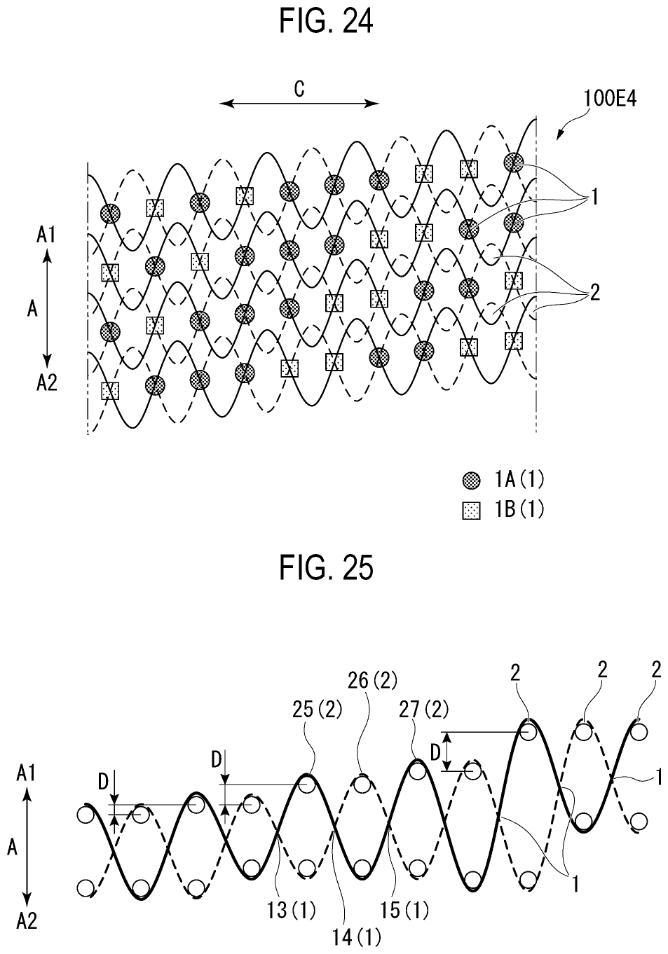

FIG. 24 is a diagram showing a stent 100E4 that is a modification of the arrangement of the first straight-line crossing portion 1A and the second straight-line crossing portion 1B. Three or two first straight-line crossing portions 1A continuous in the circumferential direction C are arranged spirally along the longitudinal axis direction A. Two second straight-line crossing portions 1B continuous in the circumferential direction C are arranged spirally along the longitudinal axis direction A.

### (Modification 5-5)

In the above embodiment, the plurality of interlocking portions 2 are spirally arranged along the longitudinal axis direction A. The distance D in the longitudinal axis direction A between the interlocking portions 2 adjacent in the circumferential direction C is substantially the same. However, the arrangement mode of the plurality of interlocking portions 2 is not limited to this. FIGS. 25A and 25B are diagrams showing a modification of the arrangement of the plurality of interlocking portions 2. The distance D in the longitudinal direction A between the interlocking portions 2 adjacent to each other in the circumferential direction C does not have to be a constant distance. Moreover, the plurality of interlocking portions 2 may be arranged in a spiral shape as a whole. The same is true for the plurality of straight-line crossing portions 1.

As shown in FIG. 25, the plurality of interlocking portions 2 include a fifth interlocking portion 25, a sixth interlocking portion 26, and a seventh interlocking portion 27. The sixth interlocking portion 26 is adjacent to the fifth interlocking portion 25 in the circumferential direction C. The seventh interlocking portion 27 is adjacent to the sixth interlocking portion 26 in the circumferential direction C. The positions of the fifth interlocking portion 25 and the sixth interlocking portion 26 in the longitudinal direction A are substantially the same, and the positions of the sixth interlocking portion 26 and the seventh interlocking portion 27 in the longitudinal direction A are different.

As shown in FIG. 25, the plurality of straight-line crossing portions 1 include a third straight-line crossing portion 13, a fourth straight-line crossing portion 1 F4, and a fifth straight-line crossing portion 15. The fourth straight-line crossing portion 14 is adjacent to the third straight-line crossing portion 13 in the circumferential direction C. The fifth straight-line crossing portion 15 is adjacent to the fourth straight-line crossing portion 14 in the circumferential direction C. The positions of the longitudinal axis direction A of the third straight-line crossing portion 13 and the fourth straight-line crossing portion 14 are substantially the same, and the positions of the longitudinal axis direction A of the fourth straight-line crossing portion 14 and the fifth straight-line crossing portion 15 are different.

### Sixth Embodiment

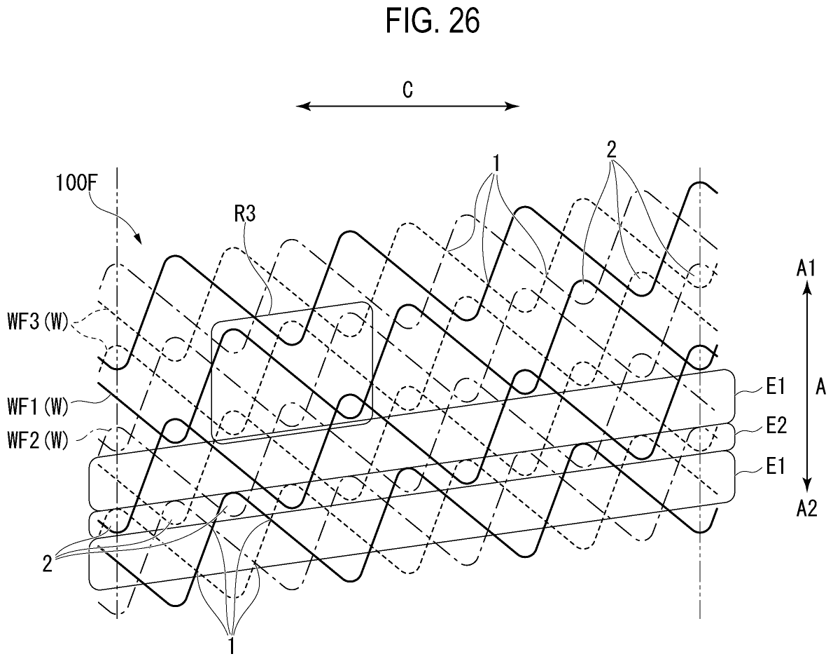

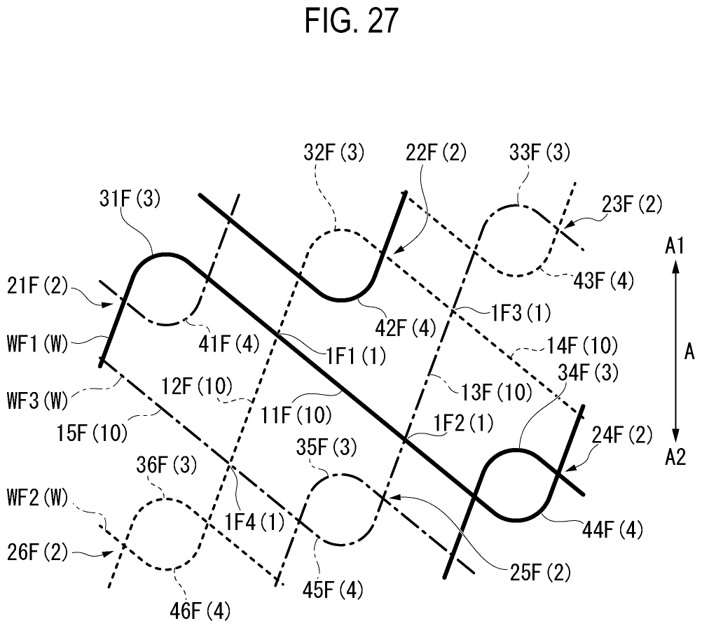

A sixth embodiment of the present invention will be described with reference to FIGS. 26 to 27. In the following description, the same reference numerals are given to the same configurations as those already described, and redundant descriptions will be omitted. A stent 100F according to the sixth embodiment is housed in a stent delivery system 150, like the stent 100 according to the first embodiment.

FIG. 26 is a developed view of the stent 100F deployed in the circumferential direction C.

The stent 100F is formed in the shape of a circular tube having meshes on its peripheral surface by means of wires W that extend obliquely in the circumferential direction C while repeatedly bending. The stent 100F has a plurality of straight-line crossing portions 1 and a plurality of the interlocking portions 2.

As shown in FIG. 26, first regions E1 in which the plurality of straight-line crossing portions 1 are arranged and second regions E2 in which the plurality of interlocking portions 2 are arranged are alternately arranged in the longitudinal axis direction A. The first region E1 is spirally arranged along the longitudinal axis direction A. In addition, the second region E2 is spirally arranged along the longitudinal axis direction A.

In the end region of the stent 100F on the second direction A2 side, the ends of the valley-shaped bent portion 4 on the second direction A2 side are arranged spirally without crossing the peak-shaped bent portion 3, as shown in FIG. 26, or may be aligned with each other in the longitudinal axis direction A. The same applies to the ends of the peak-shaped bent portions 3 on the first direction A1 side in the end regions of the stent 100F on the first direction A1 side. For example, as in the end regions of the stent 100 shown in FIG. 34 and the stent 100K shown in FIG. 38. Thus, the positions of the ends with respect to the longitudinal axis direction A can be aligned.

### [First Straight-Line Crossing Portion 1F1]

FIG. 27 is an enlarged view of region R3 shown in FIG. 26.

A first straight-line portion 11F and a second straight-line portion 12F, which are the straight-line portions 10, intersect at the “first straight-line crossing portion 1F1”, which is the straight-line crossing portion 1, when viewed from the radial direction R of the stent 100F.

### [Second Straight-Line Crossing Portion 1F2]

The first straight-line portion 11F and the third straight-line portion 13F, which are the straight-line portions 10, intersect at the “second straight-line crossing portion 1F2”, which is the straight-line crossing portion 1, when viewed from the radial direction R of the stent 100F. The second straight-line crossing portion 1F2 is arranged on the second direction A2 side with respect to the first straight-line crossing portion 1F1.

### [Third Straight-Line Crossing Portion 1F3]

The third straight-line portion 13F and the fourth straight-line portion 14F, which are the straight-line portions 10, intersect at the “third straight-line crossing portion 1F3”, which is the straight-line crossing portion 1, when viewed from the radial direction R of the stent 100F. The third straight-line crossing portion 1F3 is arranged on the first direction A1 side with respect to the second straight-line crossing portion 1F2.

### [Fourth Straight-Line Crossing Portion 1F4]

The second straight-line portion 12F and the fifth straight-line portion 15F, which are the straight-line portions 10, intersect at the “fourth straight-line crossing portion 1F4”, which is the straight-line crossing portion 1, when viewed from the radial direction R of the stent 100F. The fourth straight-line crossing portion 1F4 is arranged on the second direction A2 side with respect to the first straight-line crossing portion 1F1.

The first straight-line crossing portion 1F1, the second straight-line crossing portion 1F2, the third straight-line crossing portion 1F3, and the fourth straight-line crossing portion 1F4 are arranged in the same first region E1.

### [First Interlocking Portion 21F]

A “first peak 31F”, which is the peak-shaped bent portion 3, is connected to the first direction A1 side of the first straight-line portion 11F. The first peak 31F intersects with the first valley 41F, which is the valley-shaped bent portion 4, to form the “first interlocking portion (upper interlocking portion) 21F”, which is the interlocking portion 2.

### [Second Interlocking Portion 22F]

The “second peak 32F”, which is the peak-shaped bent portion 3, is connected to the first direction A1 side of the second straight-line portion 12F and the fourth straight-line portion 14F. The second peak 32F intersects with the second valley 42F, which is the valley-shaped bent portion 4, to form the “second interlocking portion 22F”, which is the interlocking portion 2.

### [Third Interlocking Portion 23F]

The “third peak 33F”, which is the peak-shaped bent portion 3, continues on the first direction A1 side of the third straight-line portion 13F. The third peak 33F intersects with the third valley 43F, which is the valley-shaped bent portion 4, to form the “third interlocking portion 23F”, which is the interlocking portion 2.

### [Fourth Interlocking Portion 24F]

The “fourth valley 44F”, which is the valley-shaped bent part 4, is connected to the second direction A2 side of the first straight-line portion 11F. The fourth valley 44F intersects with the fourth peak 34F, which is the peak-shaped bent portion 3, to form the “fourth interlocking portion (lower interlocking portion) 24F”, which is the interlocking portion 2.

### [Fifth Interlocking Portion 25F]

The “fifth valley 45F”, which is the valley-shaped bent part 4, continues on the second direction A2 side of the third straight-line portion 13F and the fifth straight-line portion 15F. The fifth valley 45F intersects with the fifth peak 35F, which is the peak-shaped bent portion 3, to form the “fifth hook portion 25F”, which is the hook portion 2.

### [Sixth Interlocking Portion 26F]

A “sixth valley 46F”, which is the valley-shaped bent part 4, continues on the second direction A2 side of the second straight-line portion 12F. The sixth valley 46F intersects with the sixth peak 36F, which is the peak-shaped bent portion 3, to form the “sixth interlocking portion 26F”, which is the interlocking portion 2.

### [Crossing Portion of First Straight-Line Portion 11F]

The first straight-line portion 11F is connected to the first interlocking portion (upper interlocking portion) 21F on the first direction A1 side and to the fourth interlocking portion (lower interlocking portion) 24F on the second direction A2 side. The first straight-line portion 11F is located between the first interlocking portion (upper interlocking portion) 21F and the fourth interlocking portion (lower interlocking portion) 24F, and the other two straight-line portions 10 (the second straight-line portion 12F, the third 13F) and straight-line crossing portions 1 (first straight-line crossing portion 1F1, second straight-line crossing portion 1F2).

### [Arrangement of First Interlocking Portion 21F, Second Interlocking Portion 22F, and Third Interlocking Portion 23F]

The first interlocking portion 21F, the second interlocking portion 22F, and the third interlocking portion 23F are arranged along the circumferential direction C and arranged in the same second region E2.

The first interlocking portion 21F and the second interlocking portion 22F are adjacent to each other in the circumferential direction C and arranged at different positions in the longitudinal direction A. Specifically, the second interlocking portion 22F is arranged on the first direction A1 side in the longitudinal axis direction A from the first interlocking portion 21F.

The second interlocking portion 22F and the third interlocking portion 23F are adjacent to each other in the circumferential direction C and arranged at different positions in the longitudinal direction A. Specifically, the third interlocking portion 23F is arranged on the first direction A1 side in the longitudinal axis direction A from the second interlocking portion 22F.

### [Arrangement of Fourth Interlocking Portion 24F, Fifth Interlocking Portion 25F, and Sixth Interlocking Portion 26F]

The fourth interlocking portion 24F, the fifth interlocking portion 25F, and the sixth interlocking portion 26F are arranged along the circumferential direction C and arranged in the same second region E2.

The fourth interlocking portion 24F and the fifth interlocking portion 25F are adjacent to each other in the circumferential direction C and arranged at different positions in the longitudinal direction A. Specifically, the fifth interlocking portion 25F is arranged on the second direction A2 side in the longitudinal axis direction A from the fourth interlocking portion 24F.

The fifth interlocking portion 25F and the sixth interlocking portion 26F are adjacent to each other in the circumferential direction C and arranged at different positions in the longitudinal direction A. Specifically, the sixth interlocking portion 26F is arranged on the second direction A2 side in the longitudinal axis direction A from the fifth interlocking portion 25F.

### [Wire W]

The first peak 31F, the first straight-line portion 11F, and the fourth valley 44F are a continuous part of the wire W extending in a zigzag along the circumferential direction C (first wire WF1). The sixth valley 46F, the second straight-line portion 12F, the second peak 32F, and the fourth straight-line portion 14F are continuous parts of the wire W extending in a zigzag along the circumferential direction C (second wire WF2). Further, the fifth straight-line portion 15F, the fifth valley 45F, the third straight-line portion 13F, and the third peak 33F are continuous parts (third wire WF3) of the wire W extending in a zigzag along the circumferential direction C. The first wire WF1, the second wire WF2 and the third wire WF3 may be one continuous wire or may be different wires.

In this embodiment, the second valley 42F and the fourth peak 34F are formed by the first wire WF1, the third valley 43F and the sixth peak 36F are formed by the second wire WF2, and the first valley 41F and the fifth peak 35F are formed by the third wire WF3.

### [Other Straight-Line Crossing Portions 1 and Interlocking Portions 2]

The straight-line crossing portion 1 (first straight-line crossing portion 1F1, second straight-line crossing portion 1F2, third straight-line crossing portion 1F3 and fourth straight-line crossing portion 1F4), and six interlocking portions 2 (the first interlocking portion 21F, the second interlocking portion 22F, the third interlocking portion 23F, the fourth interlocking portion 24F, the fifth interlocking portion 25F and the sixth interlocking portion 26F) have the above configuration. As shown in FIG. 26, the interlocking portion 2 connected to another straight-line crossing portion 1 in the stent 100F has the same configuration as the above configuration.

The stent 100F of the present embodiment includes a plurality of interlocking portions 2, and has high shape followability even when bent. In the stent 100F, since it is not necessary to arrange the straight-line crossing portion 1 at a position adjacent to the interlocking portion 2 in the circumferential direction C, the force (axial force) against bending is reduced. Furthermore, since the stent 100F has more straight-line crossing portions 1 than the stent 100 of the first embodiment, friction of the strut is likely to occur between the straight-line crossing portion 1 and the interlocking portion 2 when the entire stent is bent, and the shape retention is high.

The sixth embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### Seventh Embodiment

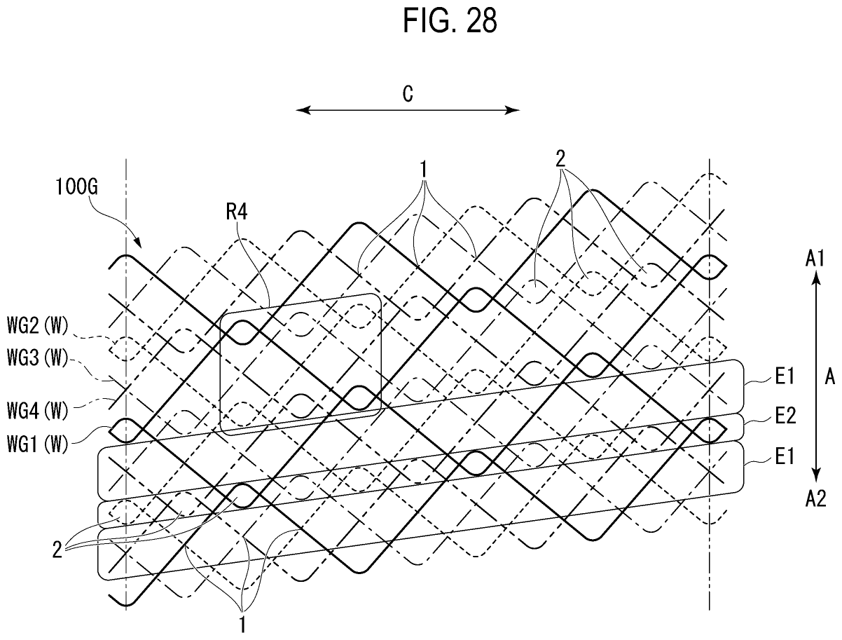

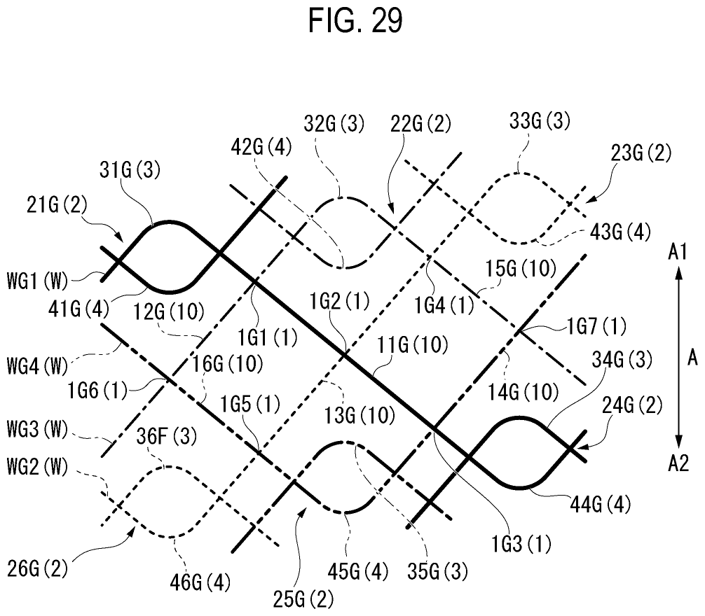

A seventh embodiment of the present invention will be described with reference to FIGS. 28 to 29. In the following description, the same reference numerals are given to the same configurations as those already described, and redundant descriptions will be omitted. A stent 100G according to the seventh embodiment is accommodated in a stent delivery system 150, like the stent 100 according to the first embodiment.

FIG. 28 is a developed view of the stent 100G deployed in the circumferential direction C.

The stent 100G is formed in the shape of a circular tube having meshes on its peripheral surface by means of wires W that extend obliquely in the circumferential direction C while repeating bending. The stent 100G has a plurality of straight-line crossing portions 1 and a plurality of interlocking portions 2.

As shown in FIG. 28, the first regions E1 in which the plurality of straight-line crossing portions 1 are arranged and the second regions E2 in which the plurality of interlocking portions 2 are arranged are alternately arranged in the longitudinal axis direction A. The first region E1 is spirally arranged along the longitudinal axis direction A. In addition, the second region E2 is spirally arranged along the longitudinal axis direction A.

In the end region of the stent 100G on the second direction A2 side, the ends of the valley-shaped bent portion 4 on the second direction A2 side are arranged in a spiral shape without crossing the peak-shaped bent portion 3, as shown in FIG. 28, or may be aligned with each other in the longitudinal axis direction A. The same applies to the ends of the peak-shaped bent portions 3 on the first direction A1 side in the end regions of the stent 100G on the first direction A1 side. For example, as in the end regions of the stent 100 shown in FIG. 34 and the stent 100K shown in FIG. 38. Thus, the positions of the ends with respect to the longitudinal axis direction A can be aligned.

### [First Straight-Line Crossing Portion 1G1]

FIG. 29 is an enlarged view of region R4 shown in FIG. 28.

The first straight-line portion 11G and the second straight-line portion 12G, which are the straight-line portions 10, intersect at the “first straight-line crossing portion 1G1”, which is the straight-line crossing portion 1, when viewed from the radial direction R of the stent 100G.

### [Second Straight-Line Crossing Portion 1G2]

The first straight-line portion 11G and the third straight-line portion 13G, which are the straight-line portions 10, intersect at the “second straight-line crossing portion 1G2”, which is the straight-line crossing portion 1, when viewed from the radial direction R of the stent 100G. The second straight-line crossing portion 1G2 is arranged on the second direction A2 side with respect to the first straight-line crossing portion 1G1.

### [Third Straight-Line Crossing Portion 1G3]

The first straight-line portion 11G and the fourth straight-line portion 14G, which are the straight-line portions 10, intersect at the “third straight-line crossing portion 1G3”, which is the straight-line crossing portion 1, when viewed from the radial direction R of the stent 100G. The third straight-line crossing portion 1G3 is arranged on the second direction A2 side with respect to the second straight-line crossing portion 1G2.

### [Fourth Straight-Line Crossing Portion 1G4]

The third straight-line portion 13G and the fifth straight-line portion 15G, which are the straight-line portions 10, intersect at the “fourth straight-line crossing portion 1G4”, which is the straight-line crossing portion 1, when viewed from the radial direction R of the stent 100G. The fourth straight-line crossing portion 1G4 is arranged on the first direction A1 side with respect to the second straight-line crossing portion 1G2.

### [Fifth Straight-Line Crossing Portion 1G5]

The third straight-line portion 13G and the sixth straight-line portion 16G, which are the straight-line portions 10, intersect at the “fifth straight-line crossing portion 1G5”, which is the straight-line crossing portion 1, when viewed from the radial direction R of the stent 100G. The fifth straight-line crossing portion 1G5 is arranged on the second direction A2 side with respect to the second straight-line crossing portion 1G2.

### [Sixth Straight-Line Crossing Portion 1G6]

The second straight-line portion 12G and the sixth straight-line portion 16G, which are the straight-line portions 10, intersect at the “sixth straight-line crossing portion 1G6”, which is the straight-line crossing portion 1, when viewed from the radial direction R of the stent 100G. The sixth straight-line crossing portion 1G6 is arranged on the second direction A2 side with respect to the first straight-line crossing portion 1G1 and on the first direction A1 side with respect to the fifth straight-line crossing portion 1G5.

### [Seventh Straight-Line Crossing Portion 1G7]

The fourth straight-line portion 14G and the fifth straight-line portion 15G, which are the straight-line portions 10, intersect at the “seventh straight-line crossing portion 1G7”, which is the straight-line crossing portion 1, when viewed from the radial direction R of the stent 100G. The seventh straight-line crossing portion 1G7 is arranged on the second direction A2 side with respect to the fourth straight-line crossing portion 1G4 and on the first direction A1 side with respect to the third straight-line crossing portion 1G3.

The first straight-line crossing portion 1G1, the second straight-line crossing portion 1G2, the third straight-line crossing portion 1G3, the fourth straight-line crossing portion 1G4, the fifth straight-line crossing portion 1G5, the sixth straight-line crossing portion 1G6 and the seventh straight-line crossing portion 1G7 are arranged in the same first region E1.

### [First Interlocking Portion 21G]

A “first peak 31G”, which is the peak-shaped bent portion 3, is connected to the first direction A1 side of the first straight-line portion 11G. The first peak 31G intersects with the first valley 41G, which is the valley-shaped bent portion 4, to form the “first interlocking portion (upper interlocking portion) 21G”, which is the interlocking portion 2.

### [Second Interlocking Portion 22G]

A “second peak 32G”, which is the peak-shaped bent portion 3, is connected to the first direction A1 side of the second straight-line portion 12G and the fifth straight-line portion 15G. The second peak 32G intersects with the second valley 42G, which is the valley-shaped bent portion 4, to form the “second interlocking portion 22G”, which is the interlocking portion 2.

### [Third Interlocking Portion 23G]

A “third peak 33G”, which is the peak-shaped bent portion 3, is connected to the first direction A1 side of the third straight-line portion 13G. The third peak 33G intersects with the third valley 43G, which is the valley-shaped bent portion 4, to form the “third interlocking portion 23G”, which is the interlocking portion 2.

### [Fourth Interlocking Portion 24G]

The “fourth valley 44G”, which is the valley-shaped bent part 4, is connected to the second direction A2 side of the first straight-line portion 11G. The fourth valley 44G intersects with the fourth peak 34G, which is the peak-shaped bent portion 3, to form the “fourth interlocking portion (lower interlocking portion) 24G”, which is the engaging section 2.

### [Fifth Interlocking Portion 25G]

A “fifth valley 45G”, which is the valley-shaped bent part 4, continues on the second direction A2 side of the fourth straight-line portion 14G and the sixth straight-line portion 16G. The fifth valley 45G intersects with the fifth peak 35G, which is the peak-shaped bent portion 3, to form the “fifth hook portion 25G”, which is the hook portion 2.

### [Sixth Interlocking Portion 26G]

A “sixth valley 46G”, which is the valley-shaped bent part 4, continues on the second direction A2 side of the third straight-line portion 13G. The sixth valley 46G intersects with the sixth peak 36G, which is the peak-shaped bent portion 3, to form the “sixth interlocking portion 26G”, which is the interlocking portion 2.

### [Crossing Portion of First Straight-Line Portion 11G]

The first straight-line portion 11G connects with the first interlocking portion (upper interlocking portion) 21G on the first direction A1 side, and connects with the fourth interlocking portion (lower interlocking portion) 24G on the second direction A2 side. Between the first interlocking portion (upper interlocking portion) 21G and the fourth interlocking portion (lower interlocking portion) 24G, the first straight-line portion 11G and the other three straight-line portions 10 (the second straight-line portion 12G, the third straight-line portion 13G, fourth straight-line portion 14G) constitute the straight-line crossing portions 1 (first straight-line crossing portion 1G1, second straight-line crossing portion 1G2, third straight-line crossing portion 1G3), respectively.

### [Arrangement of First Interlocking Portion 21G, Second Interlocking Portion 22G, and Third Interlocking Portion 23G]

The first interlocking portion 21G, the second interlocking portion 22G, and the third interlocking portion 23G are arranged along the circumferential direction C and arranged in the same second region E2.

The first interlocking portion 21G and the second interlocking portion 22G are adjacent to each other in the circumferential direction C and arranged at different positions in the longitudinal direction A. Specifically, the second interlocking portion 22G is arranged on the first direction A1 side in the longitudinal axis direction A from the first interlocking portion 21G.

The second interlocking portion 22G and the third interlocking portion 23G are adjacent to each other in the circumferential direction C and arranged at different positions in the longitudinal direction A. Specifically, the third interlocking portion 23G is arranged on the first direction A1 side in the longitudinal axis direction A from the second interlocking portion 22G.

### [Arrangement of Fourth Interlocking Portion 24G, Fifth Interlocking Portion 25G, and Sixth Interlocking Portion 26G]

The fourth interlocking portion 24G, the fifth interlocking portion 25G, and the sixth interlocking portion 26G are arranged along the circumferential direction C and arranged in the same second region E2.

The fourth interlocking portion 24G and the fifth interlocking portion 25G are adjacent to each other in the circumferential direction C and arranged at different positions in the longitudinal direction A. Specifically, the fifth interlocking portion 25G is arranged on the second direction A2 side in the longitudinal axis direction A from the fourth interlocking portion 24G.

The fifth interlocking portion 25G and the sixth interlocking portion 26G are adjacent to each other in the circumferential direction C and arranged at different positions in the longitudinal direction A. Specifically, the sixth interlocking portion 26G is arranged on the second direction A2 side in the longitudinal axis direction A from the fifth interlocking portion 25G.

### [Wire W]

The first peak 31G, the first straight-line portion 11G, and the fourth valley 44G are a continuous part of the wire W extending in a zigzag along the circumferential direction C (first wire WG1). Also, the sixth valley 46G, the third straight-line portion 13G, and the third peak 33G are a continuous part of the wire W extending in a zigzag along the circumferential direction C (second wire WG2). Further, the second straight-line portion 12G, the second peak 32G, and the fifth straight-line portion 15G are continuous parts of the wire W extending in a zigzag along the circumferential direction C (third wire WG3). The sixth straight-line portion 16G, the fifth valley 45G, and the fourth straight-line portion 14G are continuous portions of the wire W extending in a zigzag along the circumferential direction C (fourth wire WG4). The first wire WG1, the second wire WG2, the third wire WG3, and the fourth wire WG4 may be one continuous wire, or may be different wires.

In this embodiment, the first valley 41G and the fourth peak 34G are formed by the first wire WG1, the third valley 43G and the sixth peak 36G are formed by the second wire WG2, the second valley 42G is formed by the third wire WG3, and the fifth peak 35G is formed by the fourth wire WG4.

### [Other Straight-Line Crossing Portions 1 and Interlocking Portions 2]

The straight-line crossing portion 1 (first straight-line crossing portion 1G1, second straight-line crossing portion 162, third straight-line crossing portion 1G3, fourth straight-line crossing portion 1G4, fifth straight-line crossing portion 1G5, sixth straight-line crossing portion 1G6 and seventh straight-line crossing portion 1G7) and the six interlocking portions 2 (first interlocking portion 21G, second interlocking portion 22G, third interlocking portion 23G, fourth interlocking portion 24G, fifth interlocking portion 25G and sixth interlocking portion 26G) connected to the straight-line crossing portion 1 are configured as described above. As shown in FIG. 28, the interlocking portion 2 connected to another straight-line crossing portion 1 in the stent 100G has the same configuration as the above configuration.

According to the stent 100G of the present embodiment, it has a plurality of interlocking portions 2 and has high shape followability even when bent. In the stent 100G, since it is not necessary to arrange the straight-line crossing portion 1 at a position adjacent to the interlocking portion 2 in the circumferential direction C, the force (axial force) against bending is reduced. Furthermore, since the stent 100G has more straight-line crossing portions 1 than the stent 100F of the sixth embodiment, when the stent as a whole is bent, strut friction is likely to occur between the linear crossing portion 1 and the interlocking portion 2, resulting in higher shape retention.

The seventh embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### (Modification 7-1)

In the sixth embodiment, between the first interlocking portion (upper interlocking portion) 21F and the fourth interlocking portion (lower interlocking portion) 24F, the first straight-line portion 11F and the other two straight-line portions 10 (the second straight-line portion 12F and the third straight-line portion 13F) constitute the straight-line crossing portions 1 (the first straight-line crossing portion 1F1 and the second straight-line crossing portion 1F2) respectively. In the seventh embodiment, between the first interlocking portion (upper interlocking portion) 21G and the fourth interlocking portion (lower interlocking portion) 24G, the first straight line portion 11G and the other three straight line portions 10 (second straight-line portion 12G, third straight-line portion 13G, fourth straight-line portion 14G) respectively constitute the straight-line portion portions 1 (the first straight-line portion 1G1, the second straight-line portion 1G2, and the third straight-line portion 1G3). However, the aspect of the first straight-line portion is not limited to this. The first straight-line portion may form the straight-line crossing portions 1 with four or more other straight-line portions 10 between the first interlocking portion (upper interlocking portion) and the fourth interlocking portion (lower interlocking portion).

### (Modification 7-2)

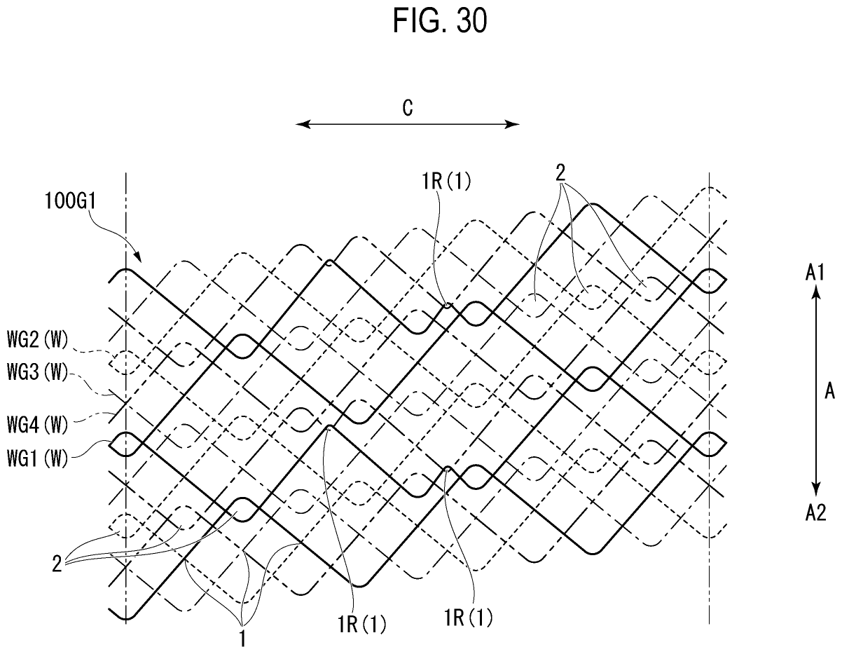

FIG. 30 is an exploded view of a stent 100G1 that is a modification of the stent 100G.

In the stent 100G1, the straight-line crossing portion 1 of the stent 100G is replaced with an interlocking portion 2 (hereinafter also referred to as “replacement interlocking portion 1R”). For example, by changing the routes of the first wire WG1 and the fourth wire WG4 as shown in FIG. 30, a part of the straight-line crossing portion 1 can be replaced with the replacement interlocking portion 1R.

In the end region of the stent 100G1 on the second direction A2 side, the ends of the valley-shaped bent portion 4 on the second direction A2 side are arranged in a spiral shape without crossing the peak-shaped bent portion 3 as shown in FIG. 30, or may be aligned with each other in the longitudinal axis direction A. In addition, the same applies to the ends of the peak-shaped bent portions 3 on the first direction A1 side in the end regions of the stent 100G1 on the first direction A1 side. For example, as in the end regions of the stent 100 shown in FIG. 34 and the stent 100K shown in FIG. 38. Thus, the positions of the ends with respect to the longitudinal axis direction A can be aligned.

### Eighth Embodiment

An eighth embodiment of the present invention will be described with reference to FIG. 1n the following description, the same reference numerals are given to the same configurations as those already described, and redundant descriptions will be omitted. A stent 100H according to the eighth embodiment is housed in a stent delivery system 150, like the stent 100 according to the first embodiment.

FIG. 31 is a developed view of the stent 100H deployed in the circumferential direction C.

The stent 100H is formed in the shape of a circular tube having meshes on its peripheral surface by means of wires W that extend obliquely in the circumferential direction C while repeatedly bending. The stent 100H has a plurality of straight-line crossing portions 1 and a plurality of interlocking portions 2.

As shown in FIG. 31, the first regions E1 in which the plurality of straight-line crossing portions 1 are arranged and the second regions E2 in which the plurality of interlocking portions 2 are arranged are alternately arranged in the longitudinal axis direction A. The first region E1 is spirally arranged along the longitudinal axis direction A. In addition, the second region E2 is spirally arranged along the longitudinal axis direction A.

As shown in FIG. 31, in the state of being developed in the circumferential direction C, the first region E1 and the second region E2, which are alternately arranged along the longitudinal axis direction A toward the second direction A2, are distinguished as second region E2(0), first region E1(1), second region E2(1), first region E1(2), second region E2(2), first region E1(3), second region E2(3), first region E1(4) and a second region E2(4). On both sides of the longitudinal axis direction A across the second region E2(n), the first region E1(n) is arranged on the first direction A1 side, and the first region E1(n+1) is arranged on the second direction A2 side, wherein n is an integer.

In the first region E1(1), a straight-line crossing portion 1 is formed by two wires W (first wire WH1 and second wire WH2), similar to the stent 100 of the first embodiment.

In the first region E1 (2), a straight-line crossing portion 1 is formed by two wires W (third wire WH3 and fourth wire WH4), similar to the stent 100 of the first embodiment. The two wires W forming the first region E1(2) are both different from the wires W forming the first region E1(1).

In the first region E1 (3), a straight-line crossing portion 1 is formed by two wires W (first wire WH1 and second wire WH2), similar to the stent 100 of the first embodiment. The two wires W forming the first region E1(3) are both the same as the wires W forming the first region E1(1).

In the first region E1 (4), a straight-line crossing portion 1 is formed by two wires W (third wire WH3 and fourth wire WH4), like the stent 100 of the first embodiment. The two wires W forming the first region E1(4) are both the same as the wires W forming the first region E1(2).

That is, the first regions E1 arranged on both sides in the longitudinal direction A with the first region E2 interposed therebetween are formed of different wires W. In this embodiment, the first regions E1 formed of different wire groups W are alternately arranged in the longitudinal axis direction A.

In the first direction A1 side end region of the stent 100H, the ends of the peak-shaped bent portion 3 on the first direction A1 side may be spirally arranged without intersecting the valley-shaped bent portion 4 as in the E2(0) region of FIG. 31, or may be arranged so as to align their positions with respect to the longitudinal axis direction A. The same applies to the second direction A2 side end portion of the valley-shaped bent portion 4 in the second direction A2 side end portion region of the stent 100H. For example, as in the end regions of the stent 100 shown in FIG. 34 and the stent 100K shown in FIG. 38, by adjusting the lengths in the longitudinal direction A of the peak-shaped bent portion 3 and the valley-shaped bent portion 4 in the end region, the positions of the ends with respect to the longitudinal direction A can be aligned. Specifically, when there are two wire groups as in this embodiment, the ends of one end region of stent 100H can be aligned by adjusting the length of one wire group. Also, the ends of the other end region of stent 100H can be aligned by adjusting the length of the other wire group.

According to the stent 100H of the present embodiment, it has a plurality of interlocking portions 2 and has a high shape followability even when it is bent. Since the stent 100H does not need to arrange the straight-line crossing portion 1 at a position adjacent to the interlocking portion 2 in the circumferential direction C, the force (axial force) against bending is reduced.

The eighth embodiment of the present invention has been described above in detail with reference to the drawings, but the specific configuration is not limited to this embodiment, and design changes and the like are also included within the scope of the present invention. Also, the constituent elements shown in the above-described embodiment and modifications shown below can be combined as appropriate.

### (Modification 8-1)

In the above embodiment, two types of first regions E1 formed by different wire groups W are alternately arranged in the longitudinal axis direction A. However, the arrangement mode of the first regions E1 formed by different wire groups W is not limited to this. Three or more types of first regions E1 formed of different wire groups W may be alternately arranged in the longitudinal axis direction A. Two or more types of first regions E1 formed of different wire groups W may be arranged irregularly in the longitudinal axis direction A.

### Ninth Embodiment

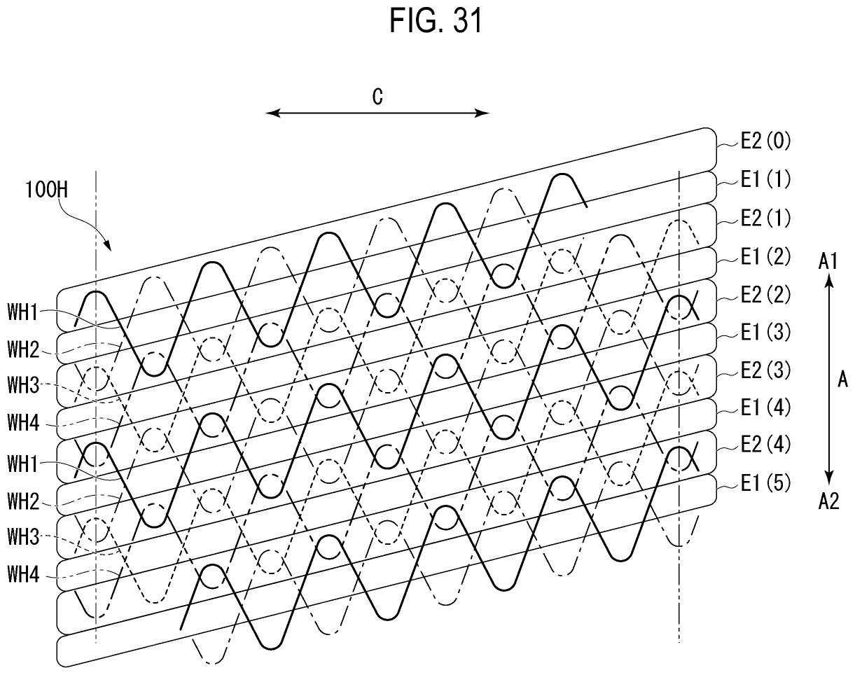

A ninth embodiment of the present invention will be described with reference to FIG. 32. In the following description, the same reference numerals are given to the same configurations as those already described, and redundant descriptions will be omitted. A stent 100I according to the ninth embodiment is housed in a stent delivery system 150, like the stent 100 according to the first embodiment.

FIG. 32 is a developed view of the stent 100I deployed in the circumferential direction C.

The stent 100I is formed in the shape of a circular tube having meshes on its peripheral surface by means of wires W that extend obliquely in the circumferential direction C while repeating bending. The stent 100I has a plurality of straight-line crossing portions 1 and a plurality of interlocking portions 2.

As shown in FIG. 32, first regions E1 in which a plurality of straight-line crossing portions 1 are arranged and second regions E2 in which a plurality of interlocking portions 2 are arranged are alternately arranged in the longitudinal axis direction A. The first region E1 is spirally arranged along the longitudinal axis direction A. In addition, the second region E2 is spirally arranged along the longitudinal axis direction A.

As shown in FIG. 32, in the state of being developed in the circumferential direction C, the first region E1 and the second region E2, which are alternately arranged in the second direction A2 along the longitudinal axis direction A, are distinguished as second region E2 (0), first region E1(1), second region E2(1), first region E1(2), second region E2(2), first region E1(3), second region E2(3), first region E1(4) and second region E2(4). On both sides of the longitudinal axis direction A across the second region E2(n), the first region E1(n) is arranged on the first direction A1 side, and the first region E1(n+1) is arranged on the second direction A2 side, wherein n is an integer.

In the first region E1(1), a straight-line crossing portion 1 is formed by two wires W (first wire WI1 and second wire WI2), like the stent 100 of the first embodiment.

In the first region E1 (2), a straight-line crossing portion 1 is formed by three wires W (the third wire WI3, the second wire WI4, and the fifth wire WI5), as in the stent 100F of the sixth embodiment. The wire W forming the first region E1(2) is different from the wire W forming the first region E1(1). The number (three) of overlapping wires W forming the first region E1(2) is different from the number (two) of overlapping wires W forming the first region E1(1).

In the first region E1(3), a straight-line crossing portion 1 is formed by two wires W (first wire WI1 and second wire WI2), like the stent 100 of the first embodiment. The two wires W forming the first region E1(3) are both the same as the wires W forming the first region E1(1). The number (two) of overlapping wires W forming the first region E1(3) is the same as the number (two) of overlapping wires W forming the first region E1(1).

In the first region E1 (4), a straight-line crossing portion 1 is formed by three wires W (third wire WI3, second wire WI4, and fifth wire WI5) in the same manner as the stent 100F of the sixth embodiment. The three wires W forming the first region E1(4) are the same as the wires W forming the first region E1(2). The number (three) of overlapping wires W forming the first region E1(4) is the same as the number (three) of overlapping wires W forming the first region E1(3).

That is, the first regions E1 arranged on both sides in the longitudinal direction A with the first region E2 interposed therebetween are formed of different wires W and formed by different wire weaving methods. In this embodiment, the first regions E1 formed by different weaving methods of the wires W are alternately arranged in the longitudinal axis direction A.

In the first direction A1 side end region of the stent 100I, the end portions of the first direction A1 side of the peak-shaped bent portion 3 may be arranged in a spiral shape without intersecting with the valley-shaped bent portion 4 as shown in the E2(0) region of FIG. 32, and may be arranged so as to align their positions with respect to the longitudinal axis direction A. The same applies to the second direction A2 side end portion of the valley-shaped bent portion 4 in the second direction A2 side end portion region of the stent 100I. For example, as in the end regions of the stent 100 shown in FIG. 34 and the stent 100K shown in FIG. 38, by adjusting the lengths in the longitudinal direction A of the peak-shaped bent portion 3 and the valley-shaped bent portion 4 in the end regions, the positions of the ends with respect to the longitudinal direction A can be aligned. Specifically, when there are two wire groups, as in this embodiment, the ends of one end region of stent 100I can be aligned by adjusting the length of one wire group. Also, the ends of the other end region of stent 100I can be aligned by adjusting the length of the other wire group.

According to the stent 100I of the present embodiment, it has a plurality of interlocking portions 2 and has a high shape followability even when bent. Since the stent 100I does not need to arrange the straight-line crossing portion 1 at a position adjacent to the interlocking portion 2 in the circumferential direction C, the force (axial force) against bending is reduced. Furthermore, the stent 100I can selectively change the shape retention according to the location by changing the weaving method of the wires W for each first region E1. Furthermore, the stent 100I can selectively change the cell size and the like depending on the expansion force and location. For example, stent 100I can have a smaller cell size to better accommodate ingrowth, or a larger cell size to facilitate special procedures such as stent-in-stent.

The ninth embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### (Modification 9-1)

In the above embodiment, the two types of first regions E1 formed by different weaving methods of the wires W are alternately arranged in the longitudinal axis direction A. However, the arrangement mode of the first regions E1 formed by different weaving methods of the wires W is not limited to this. Three or more types of first regions E1 formed by different weaving methods of the wires W may be alternately arranged in the longitudinal axis direction A. Two or more types of first regions E1 formed by different weaving methods of the wires W may be arranged irregularly in the longitudinal axis direction A.

### Tenth Embodiment

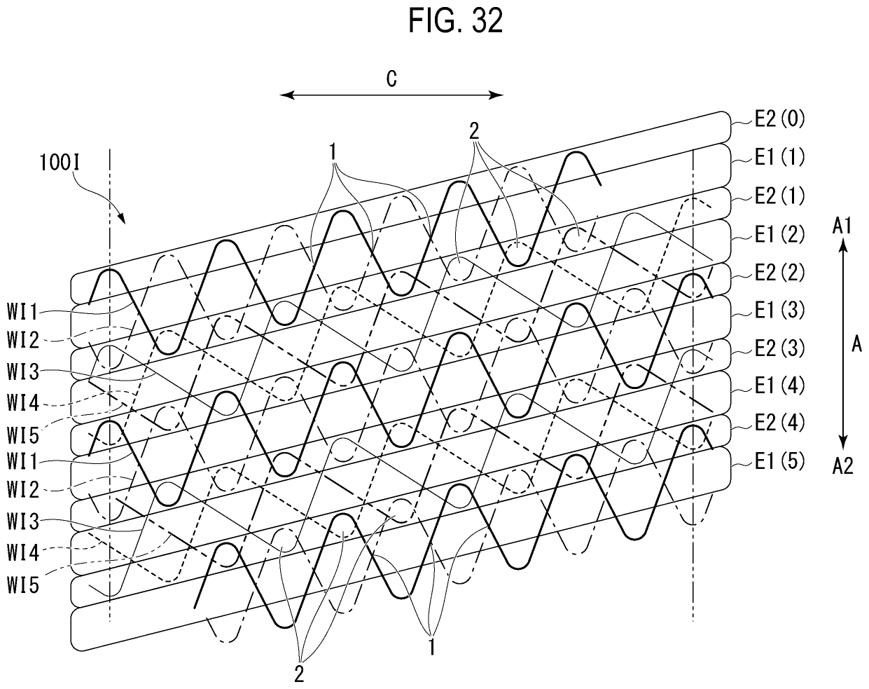

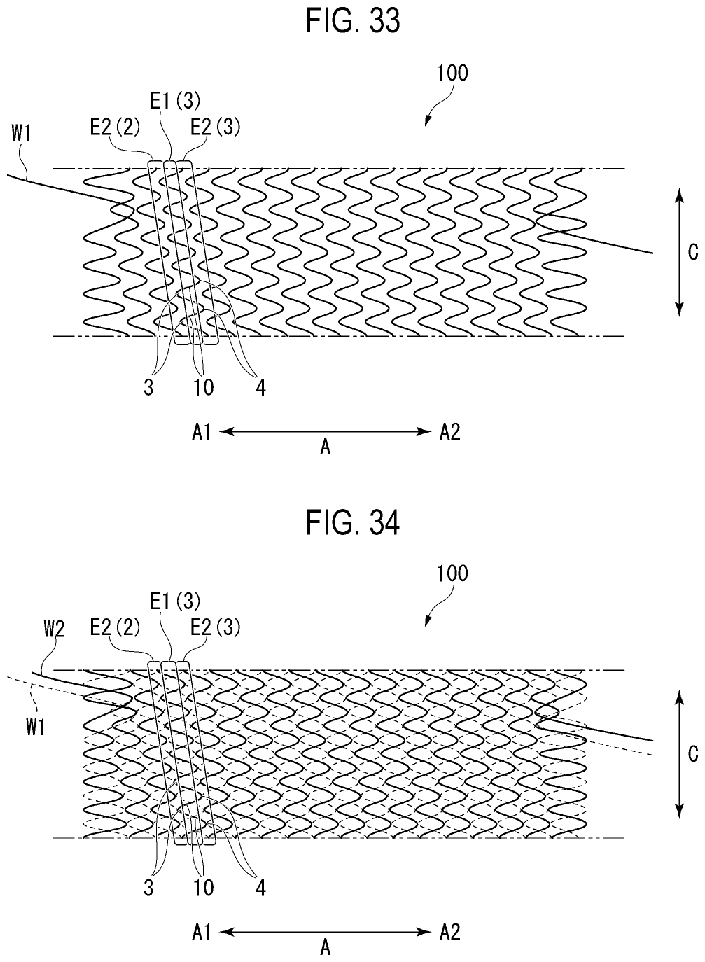

A tenth embodiment of the present invention will be described with reference to FIGS. 33 to 34. In the following description, the same reference numerals are given to the same configurations as those already described, and redundant descriptions will be omitted.

The stent manufacturing method of the present embodiment is a method of manufacturing the stent 100 according to the first embodiment, the stent 100B according to the second embodiment, the stent 100C according to the third embodiment, the stent 100D according to the fourth embodiment, and the stent 100E according to the fifth embodiment. Hereinafter, a method for manufacturing the stent 100 according to the first embodiment will be mainly described.

In the stent manufacturing method of this embodiment, the first wire W1 and the second wire W2 (see FIG. 4) forming the stent 100 are two different wires W.

In the stent manufacturing method of this embodiment, a jig as described in U.S. Pat. No. 6,974,472 is used. The jig is formed in a cylindrical shape and has a plurality of projecting pins on its outer periphery. In the jig used in this embodiment, protruding pins are provided at locations where the interlocking portions 2 of the stent 100 are formed, and are spirally arranged along the longitudinal axis direction.

### <First Wire Weaving Step>

FIG. 33 is a developed view of the knitted first wire W1 developed in the circumferential direction C.

An operator weaves the first wire W1 along a cylindrical jig having a plurality of projecting pins spirally provided. The operator weaves the first wire W1 in a zigzag pattern, so as to form a part of the interlocking portion 2 of the second region E2(n−1) and the second region E2(n) on both sides in the longitudinal direction A with the first region E1(n) interposed therebetween.

Specifically, the operator forms the peak-shaped bent portion 3 of the second region E2(n−1) by hooking the first wire W1 on the protruding pin and bending the first wire W1. Next, the operator similarly forms the valley-shaped bent portion 4 of the second region E2(n), which is continuous with the recently formed peak-shaped bent portion 3 via the straight-line portion 10. Next, the operator similarly forms the peak-shaped bent portion 3 of the second region E2(n−1), which is connected to the recently formed valley-shaped bent portion 4 via the other straight-line portion 10. Next, the operator similarly forms valley-shaped bent portions 4 in the second region E2(n), which are connected to the most recently formed peak-shaped bent portions 3 via other straight-line portions 10. Thereafter, the operator repeats this to alternately form the peak-shaped bent portions 3 and the valley-shaped bent portions 4, and knits the first wire W1 along the longitudinal axis direction A.

When the weaving of the first wire W1 is completed, as shown in FIG. 33, the wires W are not overlapped, and neither the straight-line crossing portion 1 nor the interlocking portion 2 is formed.

### <Second Wire Weaving Step>

FIG. 34 is a developed view of the knitted first wire W1 and second wire W2 developed in the circumferential direction C. The operator weaves the second wire W2 along a columnar jig provided with a plurality of projecting pins spirally. The operator weaves the second wire W2 in a zigzag pattern, so as to form the second region E2(n−1) and the rest of the interlocking portion 2 of the second region E2(n) on both sides in the longitudinal direction A across the first region E1(n).

Specifically, the operator attaches the peak-shaped bent portion 3, which is not formed by the first wire W1 and is in the second region E2(n−1), to the protruding pin with the second wire W2. is hooked to bend the second wire W2. Next, the operator similarly forms the valley-shaped bent portion 4 that is not formed by the first wire W1 and that is the valley-shaped bent portion 4 of the second region E2(n) connected to the immediately formed peak-shaped bent portion 3 via the straight-line portion 10. Next, the operator forms the peak-shaped bent portion 3 in the same manner that is not formed by first wire W1 and that is the peak-shaped bent portion 3 of the second region E2(n−1) that is connected to the valley-shaped bent portion 4 that is formed most recently via another straight portion 10. Next, the operator similarly forms the valley-shaped bent portion 4 that is not formed by the first wire W1 and that is the valley-shaped bent portion 4 of the second region E2(n) connected to the immediately formed peak-shaped bent portion 3 via another straight portion 10. Thereafter, the operator repeats this to alternately form the peak-shaped bent portions 3 and the valley-shaped bent portions 4, and knits the second wire W2 along the longitudinal axis direction A.

When weaving the second wire W2, the operator crosses the first wire W1 with the second wire W2 so that the interlocking portion 2 is formed. The peak-shaped bent portion 3 and the valley-shaped bent portion 4 intersect to form the interlocking portion 2 where the first wire W1 and the second wire W2 intersect. When the second wire W2 is woven, the operator crosses the first wire W1 with the second wire W2 so that the straight-line crossing portion 1 is formed. A straight-line crossing portion 1 is formed by the straight-line portion 10 of the first wire W1 and the straight-line portion 10 of the second wire W2 crossing each other.

### <Marker Mounting Step>

The operator attaches X-ray visibility markers to predetermined locations on the first wire W1 and the second wire W2 as necessary.

### <Washing Step>

The operator cleans the woven first wire W1 and second wire W2 as necessary.

### <Heat Treatment Step>

The operator performs heat treatment on the first wire W1 and the second wire W2 that have been woven, and performs shape memory processing on the first wire W1 and the second wire W2. The first wire W1 and the second wire W2 are, for example, a superelastic alloy whose main material is NiTi. A superelastic alloy composed mainly of NiTi is not permanently deformed when it is woven, and the woven shape is memorized by applying a heat treatment in a woven state.

### <Joining Step>

The operator joins the end of the first wire W1 and the end of the second wire W2 by caulking, laser welding, tight winding, or the like. The operator may remove the first wire W1 and the second wire W2 from the jig, and then join the end of the first wire W1 and the end of the second wire W2. The operator may join the end of the first wire W1 and the end of the second wire W2 before removing the first wire W1 and the second wire W2 from the jig.

In the stent 100 produced by the stent manufacturing method of this embodiment, an even number of interlocking portions 2 are arranged for each second region E2(n). In the stent 100 illustrated in FIG. 34, twelve interlocking portions 2 are arranged for each second region E2(n). The knitting method of the stent by the stent manufacturing method of the present embodiment is also called “even number knitting”.

In the stent 100 produced by the stent manufacturing method of this embodiment, as shown in FIG. 4, the first valleys 41 and the second peaks 32 are formed by the first wire W1. The first peak 31 and the second valley 42 are formed by the second wire W2.

The stent 100B according to the second embodiment, the stent 100C according to the third embodiment, the stent 100D according to the fourth embodiment, and the stent 100E according to the fifth embodiment can also be manufactured by the same manufacturing method.

According to the stent manufacturing method of the present embodiment, it is possible to manufacture the stent 100 or the like, which has a plurality of interlocking portions 2 and has high shape followability even when bent. As shown in FIG. 4, the first valley 41 and the second peak 32 are formed by the first wire W1, and the first peak 31 and the second valley 42 are formed by the second wire W2. Therefore, the stent 100 or the like formed by the stent manufacturing method (even-number knitting) of the present embodiment has a strong skeleton, and can reduce the occurrence of ingrowth and migration.

The tenth embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### Eleventh Embodiment

An eleventh embodiment of the present invention will be described with reference to FIGS. 35 to 38. In the following description, the same reference numerals are given to the same configurations as those already described, and redundant descriptions will be omitted.

FIG. 35 is a diagram showing a stent 100K of this embodiment.

The stent 100K differs from the stent 100 according to the first embodiment in the first wire W1 and the second wire W2. The stent 100K is formed by weaving a first wire WK1 and a second wire WK2 different from the first wire WK1.

The stent 100K is formed in the shape of a circular tube having a mesh on the peripheral surface by the first wire WK1 and the second wire WK2 extending at an angle in the circumferential direction C while repeatedly bending. The stent 100K has a plurality of straight-line crossing portions 1 and a plurality of interlocking portions 2.

The straight-line crossing portion 1 of the stent 100K is formed by crossing the straight-line portion 10 of the first wire WK1 and the straight-line portion 10 of the second wire WK2, similarly to the stent 100 according to the first embodiment.

The interlocking portion 2 of the stent 100K differs from the stent 100 according to the first embodiment in that it includes the interlocking portion 2 where the first wires WK1 cross each other (hereinafter also referred to as an interlocking portion 2K1) and the interlocking portion 2 where the second wires WK2 cross each other (hereinafter also referred to as an interlocking portion 2K2). In the stent 100K, the first peaks 31 and the first valleys 41 forming the first interlocking portion 21 shown in FIG. 4 are formed by the first wire WK1. In the stent 100K, the second peaks 32 and the second valleys 42 forming the second interlocking portion 22 shown in FIG. 4 are formed by the second wire WK2.

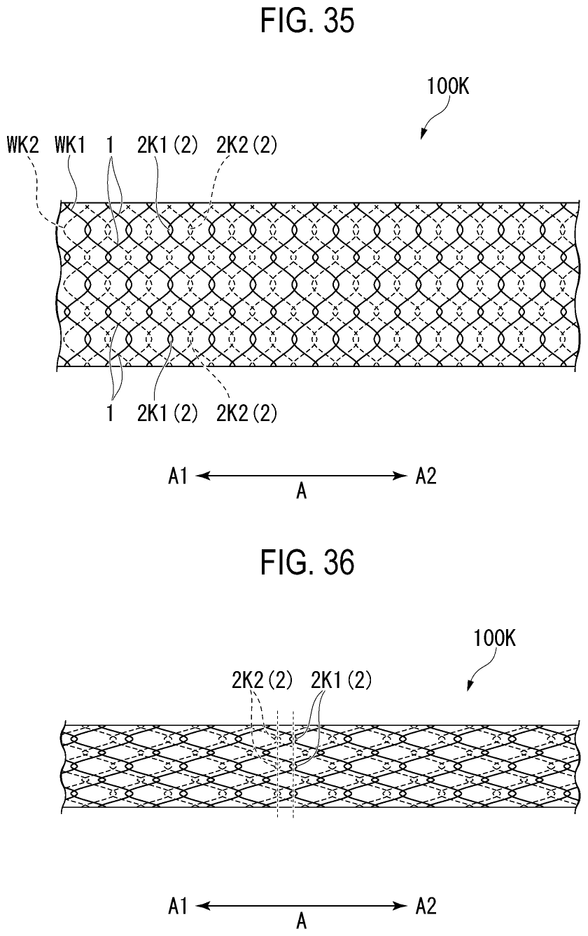

FIG. 36 is a diagram showing a stent 100K with a reduced diameter.

The first wire WK1 and the second wire WK2 that are woven into the stent 100K do not intersect at the interlocking portion 2, so they are relatively movable in the longitudinal axis direction A. Therefore, for example, as shown in FIG. 36, when the stent 100K is reduced in diameter and accommodated in the stent delivery system 150, by shifting the interlocking portion 2K1 where the first wires WK1 intersect and the interlocking portion 2K2 where the second wires WK2 intersect in the longitudinal direction A, the outer diameter of the stent 100K becomes smaller. Therefore, the stent 100K can be smoothly contracted and expanded, and can be easily released and recaptured from the stent delivery system 150.

Next, a method for manufacturing the stent 100K will be explained. In the method for manufacturing a stent of this embodiment, the same jig as in the tenth embodiment is used.

### <First Wire Weaving Step>

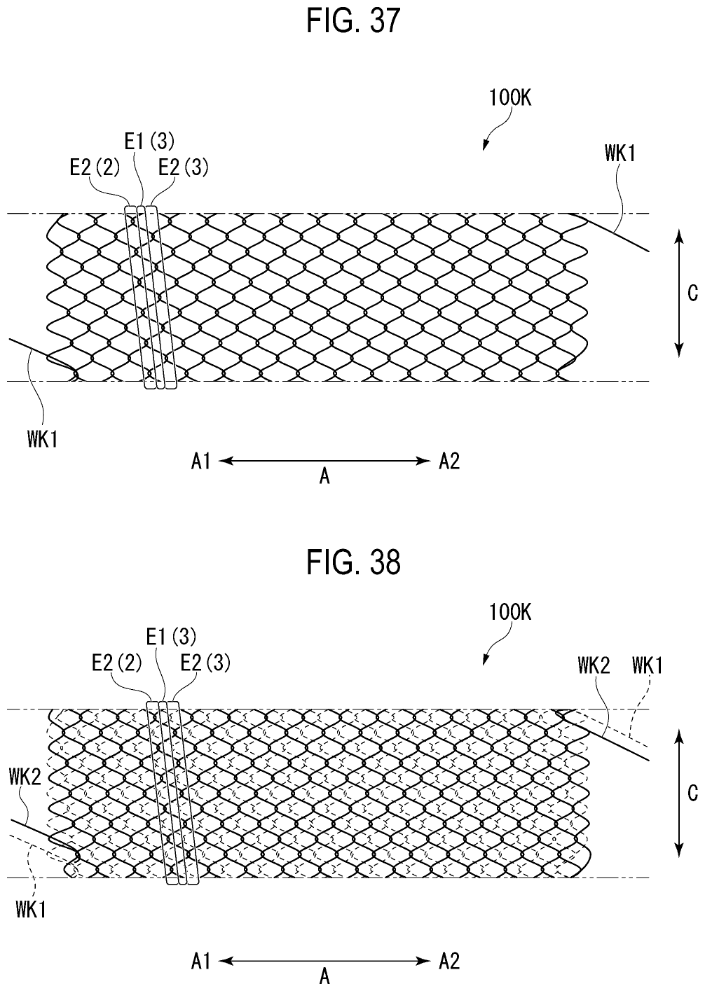

FIG. 37 is a developed view of the knitted first wire WK1 developed in the circumferential direction C.

The operator weaves the first wire WK1 along a cylindrical jig with a plurality of projecting pins spirally provided. The operator weaves the first wire WK1 in a zigzag pattern, so as to form the interlocking portion 2 of the second region E2(n−1) and the second region E2(n) on both sides in the longitudinal direction A with the first region E1(n) interposed therebetween.

Specifically, the operator forms the peak-shaped bent portion 3 of the second region E2(n−1) by hooking the first wire WK1 on the protruding pin and bending the first wire WK1. Next, the operator similarly forms the valley-shaped bent portion 4 of the second region E2(n), which is continuous with the recently formed peak-shaped bent portion 3 via the straight-line portion 10. Next, the operator similarly forms the peak-shaped bent portion 3 of the second region E2(n−1), which is connected to the recently formed valley-shaped bent portion 4 via the other straight-line portion 10. Next, the operator similarly forms valley-shaped bent portions 4 in the second region E2(n), which are connected to the most recently formed peak-shaped bent portions 3 via other straight-line portions 10. Thereafter, the operator repeats this to alternately form the peak-shaped bent portions 3 and the valley-shaped bent portions 4, and knits the first wire WK1 along the longitudinal axis direction A.

When weaving the first wires WK1, the operator crosses the first wires WK1 so that the interlocking portion 2 is formed. The peak-shaped bent portion 3 of the first wire WK1 and the valley-shaped bent portion 4 of the first wire WK1 intersect to form an interlocking portion 2K1 where the first wires WK1 intersect each other.

When the weaving of the first wire WK1 is completed, as shown in FIG. 37, the interlocking portion 2 is formed, but the straight-line crossing portion 1 is not formed.

### <Second Wire Weaving Step>

FIG. 38 is a developed view of the knitted first wire WK1 and second wire WK2 developed in the circumferential direction C. The operator weaves the second wire WK2 along a columnar jig provided with a plurality of projecting pins spirally. The operator weaves the second wire WK2 in a zigzag pattern, so as to form the interlocking portion 2 of the second region E2(n−1) and the second region E2(n) on both sides in the longitudinal direction A with the first region E1(n) interposed therebetween.

Specifically, the operator forms the peak-shaped bent portion 3 of the second region E2(n−1) by hooking the second wire WK2 on the protruding pin and bending the second wire WK2. Next, the operator similarly forms the valley-shaped bent portion 4 of the second region E2(n), which is continuous with the recently formed peak-shaped bent portion 3 via the straight-line portion 10. Next, the operator similarly forms the peak-shaped bent portion 3 of the second region E2(n−1), which is connected to the recently formed valley-shaped bent portion 4 via the other straight-line portion 10. Next, the operator similarly forms valley-shaped bent portions 4 in the second region E2(n), which are connected to the most recently formed peak-shaped bent portions 3 via other straight-line portions 10. Thereafter, the operator repeats this to alternately form the peak-shaped bent portions 3 and the valley-shaped bent portions 4, and knits the second wire WK2 along the longitudinal axis direction A.

When weaving the second wires WK2, the operator crosses the second wires WK2 so that the interlocking portion 2 is formed. The peak-shaped bent portion 3 of the second wire WK2 and the valley-shaped bent portion 4 of the second wire WK2 intersect to form an interlocking portion 2K2 where the second wires WK2 intersect each other. Further, when the operator weaves the second wire WK2, the operator crosses the first wire WK1 with the second wire WK2 so that the straight-line crossing portion 1 is formed. The straight-line crossing portion 1 is configured by the straight-line portion 10 of the first wire WK1 and the straight-line portion 10 of the second wire WK2 crossing each other.

### <Marker Mounting Step>

The operator attaches X-ray visibility markers to predetermined locations on the first wire WK1 and the second wire WK2 as necessary.

### <Washing Step>

The operator cleans the woven first wire WK1 and second wire WK2 as necessary.

### <Heat Treatment Step>

The operator performs heat treatment on the first wire WK1 and the second wire WK2 that are woven, and performs shape memory processing on the first wire WK1 and the second wire WK2. The first wire WK1 and the second wire WK2 are, for example, a superelastic alloy whose main material is NiTi. A superelastic alloy composed mainly of NiTi is not permanently deformed when it is woven, and the woven shape is memorized by applying a heat treatment in a woven state.

### <First Bonding Step>

The operator joins the end of the first wire WK1 to any part of the first wire WK1 by caulking, laser welding, tight winding, or the like. The operator may remove the first wire WK1 and the second wire WK2 from the jig and then join the ends of the first wire WK1. The operator may join the ends of the first wire WK1 before removing the first wire WK1 and the second wire WK2 from the jig.

### <Second Joining Step>

The operator joins the end of the second wire WK2 to any part of the second wire WK2 by caulking, laser welding, tight winding, or the like. The operator may remove the first wire WK1 and the second wire WK2 from the jig and then join the ends of the second wire WK2. The operator may join the ends of the second wire WK2 before removing the first wire WK1 and the second wire WK2 from the jig.

In the stent 100K produced by the stent manufacturing method of this embodiment, an odd number of interlocking portions 2 are arranged for each second region E2(n). In the stent 100K illustrated in FIG. 38, 11 interlocking portions 2 are arranged for each second region E2(n). The knitting method of the stent by the stent manufacturing method of the present embodiment is also called “odd number knitting”.

According to the stent 100K of the present embodiment, it has a plurality of interlocking portions 2 and has a high shape followability even when it is bent. Since the stent 100K does not need to arrange the straight-line crossing portion 1 at a position adjacent to the interlocking portion 2 in the circumferential direction C, the force (axial force) against bending is reduced. Furthermore, in the stent 100K, the first wire WK1 and the second wire WK2 that are woven are relatively movable in the longitudinal direction A. Therefore, the stent 100K can be smoothly contracted and expanded, and can be easily released and recaptured from the stent delivery system 150.

The eleventh embodiment of the present invention has been described above in detail with reference to the drawings, and the specific configuration is not limited to this embodiment, and design changes and the like are also included within the scope of the present invention. Also, the constituent elements shown in the above-described embodiment and modifications shown below can be combined as appropriate.

### (Modification 11-1)

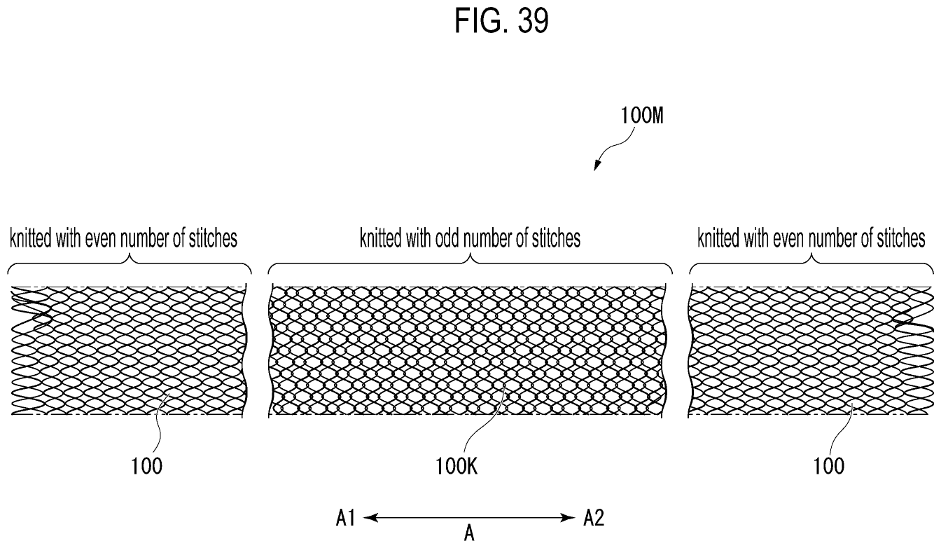

FIG. 39 shows a stent 100M that is a modification of the stent 100K.

The stent 100M is knitted with an odd number of stitches in the central portion in the longitudinal direction A, and is knitted with an even number of stitches at both ends in the longitudinal direction A. The central portion, which is knitted with an odd number of stitches, has a smooth diameter reduction operation similar to the stent 100K, and is easily captured by the stent delivery system 150. As described in the tenth embodiment, both ends knitted by even-numbered knitting have a strong skeleton and can reduce the occurrence of ingrowth and migration. By mixing two types of knitting methods (odd number knitting and even number knitting) with different properties in this way, the stent 100M can have different properties depending on the location.

### Twelfth Embodiment

A twelfth embodiment of the present invention will be described with reference to FIGS. 40 to 55. In the following description, the same reference numerals are given to the same configurations as those already described, and redundant descriptions will be omitted. A stent 100P according to the sixth embodiment is accommodated in a stent delivery system 150, like the stent 100 according to the first embodiment.

FIG. 40 is a developed view of the stent 100P deployed in the circumferential direction C.

The stent 100P is formed in the shape of a circular tube having meshes on its peripheral surface by means of wires W that extend obliquely in the circumferential direction C while repeating bending. The stent 100P has multiple interlocking portions (first interlocking portions) 2 and multiple loop interlocking portions (second interlocking portions) 2P.

The first wire W1 and the second wire extending in a zigzag along the circumferential direction C alternately form the peak-shaped bent portions 3 and the valley-shaped bent portions 4, thereby forming the interlocking portion 2 or the loop interlocking portion 2P. A second region E2 in which the interlocking portion 2 formed by the wire W extending in the circumferential direction C and the loop interlocking portion 2P are arranged is spirally arranged along the longitudinal axis direction A. Here, the second region E2 is an area divided for each round in the circumferential direction C.

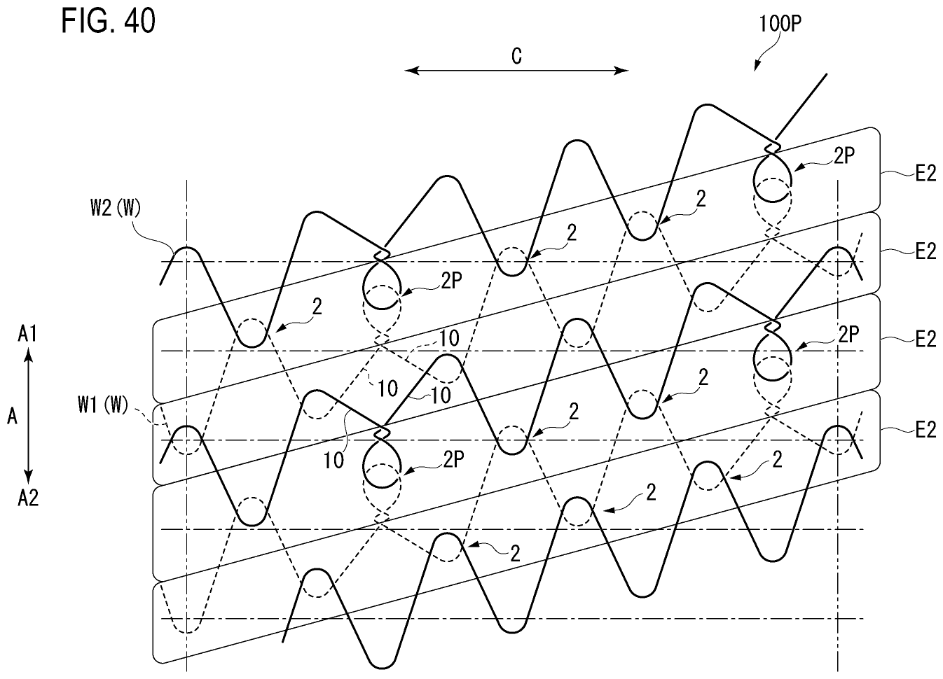

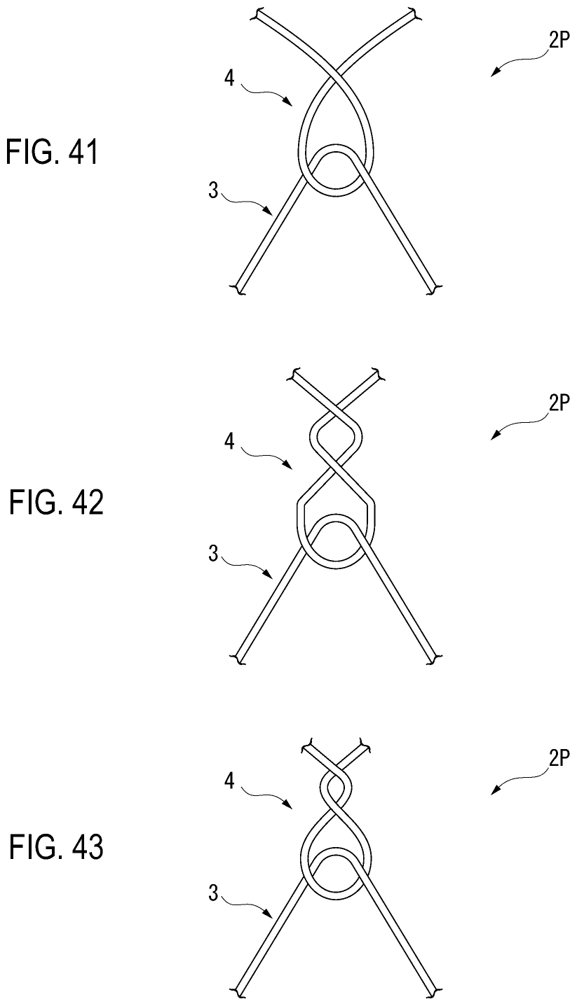

FIGS. 41 to 46 are diagrams showing the loop interlocking portion 2P.

Similarly to the interlocking portion 2, the loop interlocking portion 2P is formed by intersecting the peak-shaped bent portion 3 and the valley-shaped bent portion 4 in a hook shape. At least one of the peak-shaped bent portion 3 and the valley-shaped bent portion 4 in the loop interlocking portion 2P is formed with a loop (torsion). In the loop interlocking portion 2P, relative movement between the peak-shaped bent portion 3 and the valley-shaped bent portion 4 is regulated within a predetermined range by a loop (torsion). The loop interlocking portion 2P may be any of those shown in FIGS. 41 to 46.

A loop interlocking portion 2P shown in FIG. 41 has a single loop (one loop) formed by twisting once in the valley-shaped bent portion 4. The single loop may be formed in the peak-shaped bent portion 3.

The loop interlocking portion 2P shown in FIGS. 42 and 43 has a double loop (two loop) formed in the valley-shaped bent portion 4 by being twisted twice. The details of the double loop (two loop) will be described later. The double loop may be formed in the chevron 3.

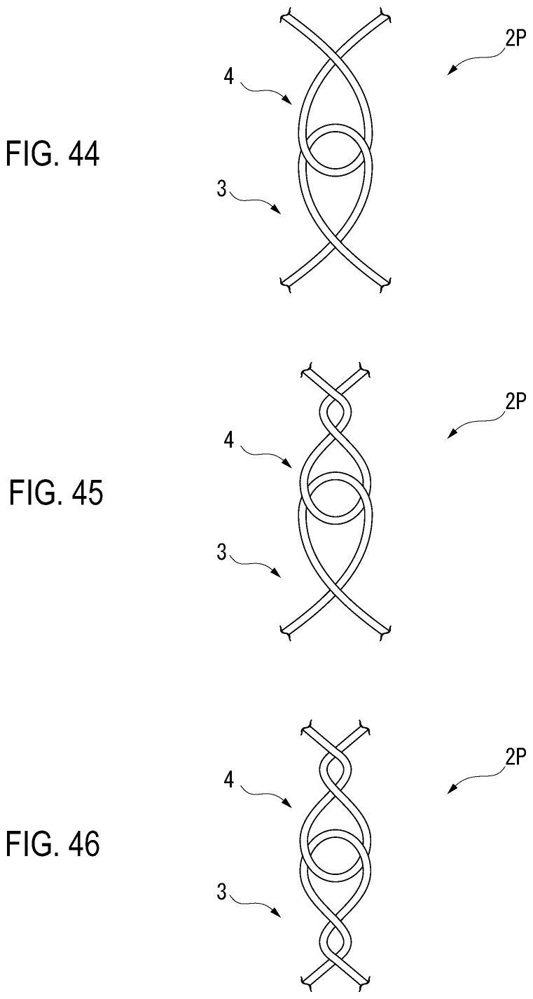

In the loop interlocking portion 2P shown in FIG. 44, a single loop (one loop) formed by twisting once is formed in the peak-shaped bent portion 3 and the valley-shaped bent portion 4.

The loop interlocking portion 2P shown in FIG. 45 has a double loop (two loop) formed in the valley-shaped bent portion 4 and a single loop (one loop) formed in the peak-shaped bent portion 3. The single loop (one loop) may be formed in the valley bent portion 4 and the double loop (two loop) may be formed in the peak bent portion 3.

In the loop interlocking portion 2P shown in FIG. 46, a double loop (two loop) formed by twisting twice are formed in the peak-shaped bent portion 3 and the valley-shaped bent portion 4.

The loop interlocking portions 2P are arranged in the longitudinal axis direction A. The loop interlocking portion 2P is connected to the interlocking portion 2 via the straight-line portion 10 of the wire W, and is not connected to the other loop interlocking portion 2P via the straight-line portion 10 of the wire W.

The second regions E2 in which the loop interlocking portions 2P are arranged and the second regions E2 in which the loop interlocking portions 2P are not arranged are alternately arranged in the longitudinal axis direction A.

According to the stent 100P of this embodiment, the second regions E2 are arranged in a spiral shape, so the stent 100P can be knitted without arranging the straight-line crossing portions 1 in the second regions E2. That is, in the stent 100P, it is not necessary to arrange the straight-line crossing portion 1 at a position adjacent to the interlocking portion 2 in the circumferential direction C. Therefore, in the second region E2, the stent 100P has many interlocking portions 2 with high shape followability, and is easy to bend. Moreover, the stent 100P includes a loop interlocking portion 2P in the second region E2. The loop interlocking portion 2P restricts relative movement between the peak-shaped bent portion 3 and the valley-shaped bent portion 4 within a predetermined range. Therefore, the stent 100P can suitably prevent the occurrence of crushing (axial crushing) in the longitudinal axis direction A.

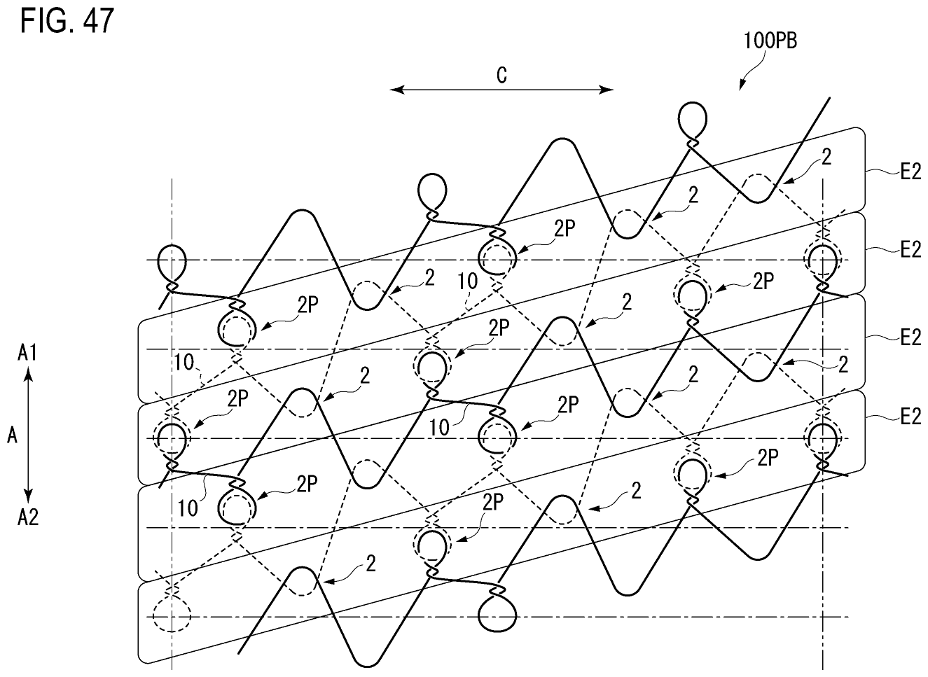

FIG. 47 is an exploded view of a stent 100 PB that is a modification of the stent 100P.

In the stent 100PB, the loop interlocking portion 2P is connected via the straight-line portion 10 of the wire W to the other two loop interlocking portions 2P. In the stent 100PB, the loop interlocking portions 2P are arranged in both of the second regions E2 adjacent in the longitudinal axis direction A. A plurality of continuous loop interlocking portions 2P connected via the straight-line portion 10 are connected along the longitudinal axis direction A in a zigzag manner. Therefore, the stent 100PB can more preferably prevent the occurrence of crushing in the longitudinal axis direction A (axial crushing) compared to the stent 100P.

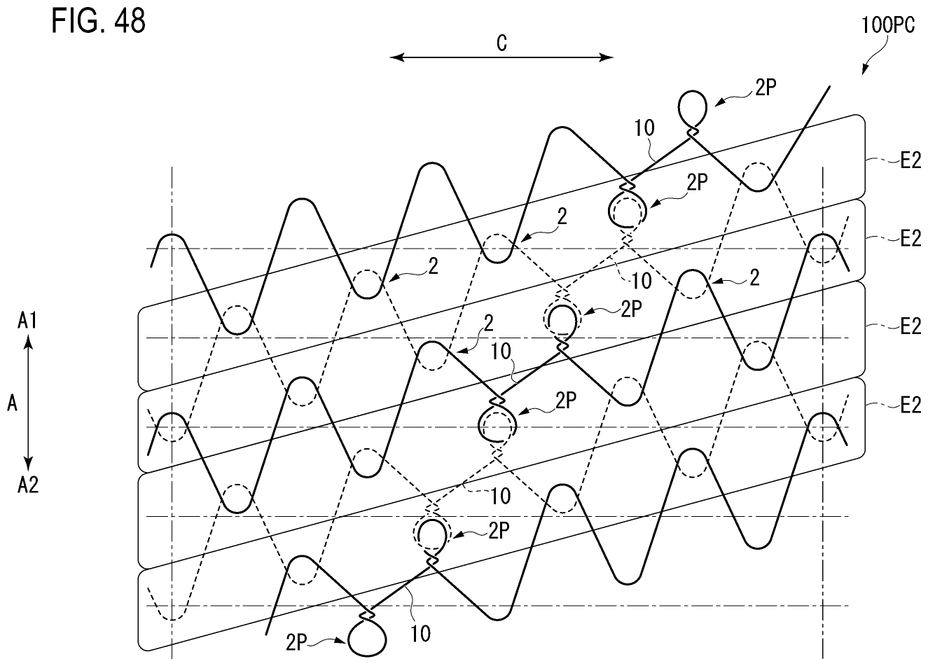

FIG. 48 is an exploded view of a stent 100PC that is a modification of the stent 100P.

In the stent 100PC, the loop interlocking portion 2P is connected via the straight-line portion 10 of the wire W to the other two loop interlocking portions 2P. In the stent 100PC, a plurality of continuous loop interlocking portions 2P connected via the straight-line portion 10 are arranged in a spiral. Therefore, the stent 100PC can more preferably prevent the occurrence of crushing (axial crushing) in the longitudinal axis direction A compared to the stent 100P.

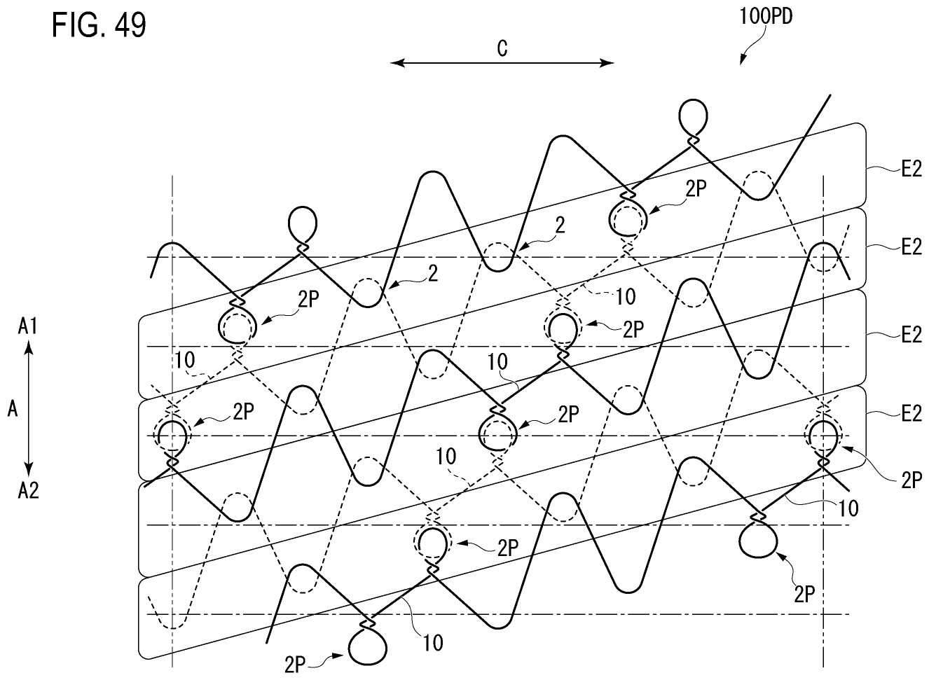

FIG. 49 is an exploded view of a stent 100PD that is a modification of the stent 100P.

In the stent 100PD, the loop interlocking portion 2P is connected via the straight-line portion 10 of the wire W to the other two loop interlocking portions 2P. In the stent 100PD, a plurality of continuous loop interlocking portions 2P connected via the straight-line portion 10 and arranged in a spiral are arranged. Therefore, the stent 100PD can more preferably prevent the occurrence of crushing in the longitudinal axis direction A (axial crushing) compared to the stent 100P.

FIG. 50 is an exploded view of a stent 100PE that is a modification of the stent 100P.

In the stent 100PE, the loop interlocking portion 2P is connected via the straight-line portion 10 of the wire W to the other two loop interlocking portions 2P. In the stent 100PE, a plurality of continuous loop interlocking portions 2P connected via the straight-line portions 10 are arranged in a spiral along the spiral direction opposite to the spiral direction in which the interlocking portions 2 are arranged in the second region E2. Therefore, the stent 100PE can more preferably prevent the occurrence of crushing (axial crushing) in the longitudinal axis direction A compared to the stent 100P.

FIG. 51 is an exploded view of a stent 100PF, which is a modification of the stent 100P.

As with the stent 100P, the stent 100PF does not connect the loop interlocking portion 2P via the straight-line portion 10 of the wire W to other loop interlocking portions 2P. Also, the loop interlocking portions 2P are arranged in the longitudinal axis direction A. The stent 100PF differs from the stent 100P in that at least one loop interlocking portion 2P is arranged in one second region E2. Therefore, the stent 100PF can maintain flexibility to some extent without excessively restricting the movement of the loop interlocking portion 2P in the longitudinal axis direction A while suppressing the occurrence of crushing (axial crushing) in the longitudinal axis direction A compared with the stent 100P.

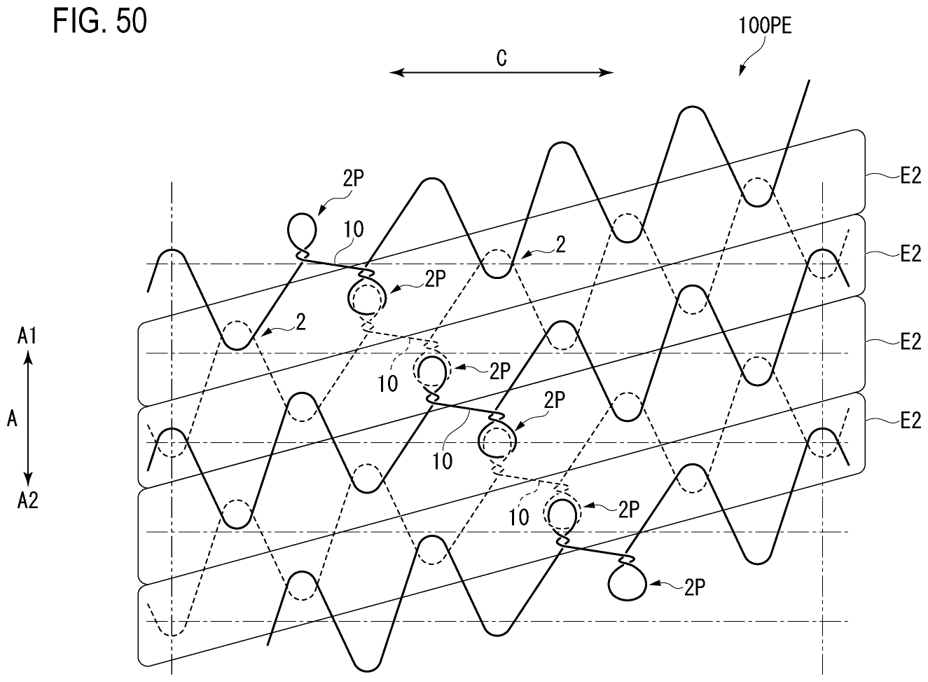

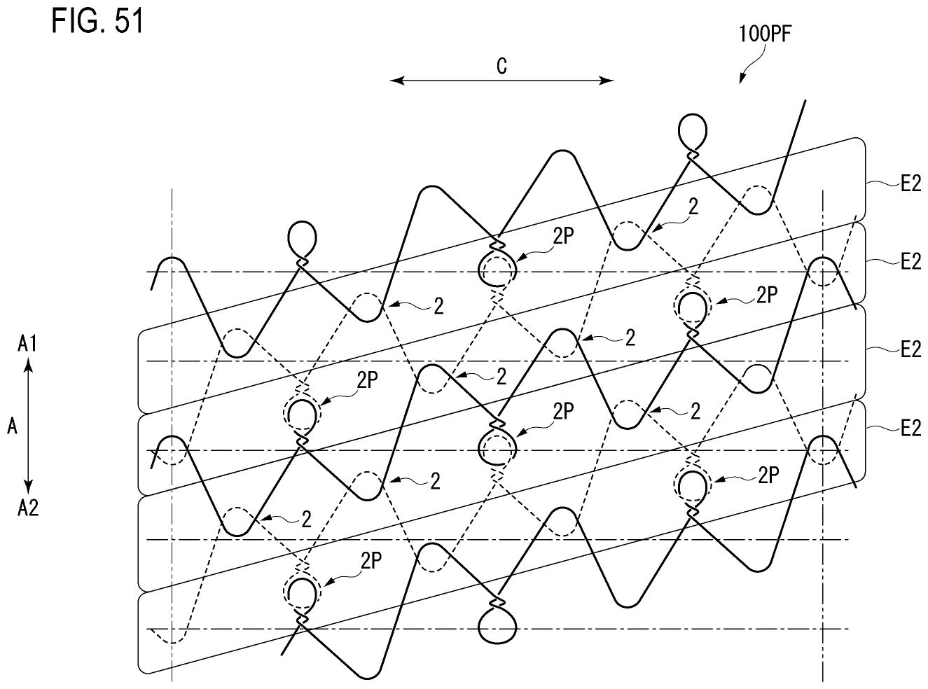

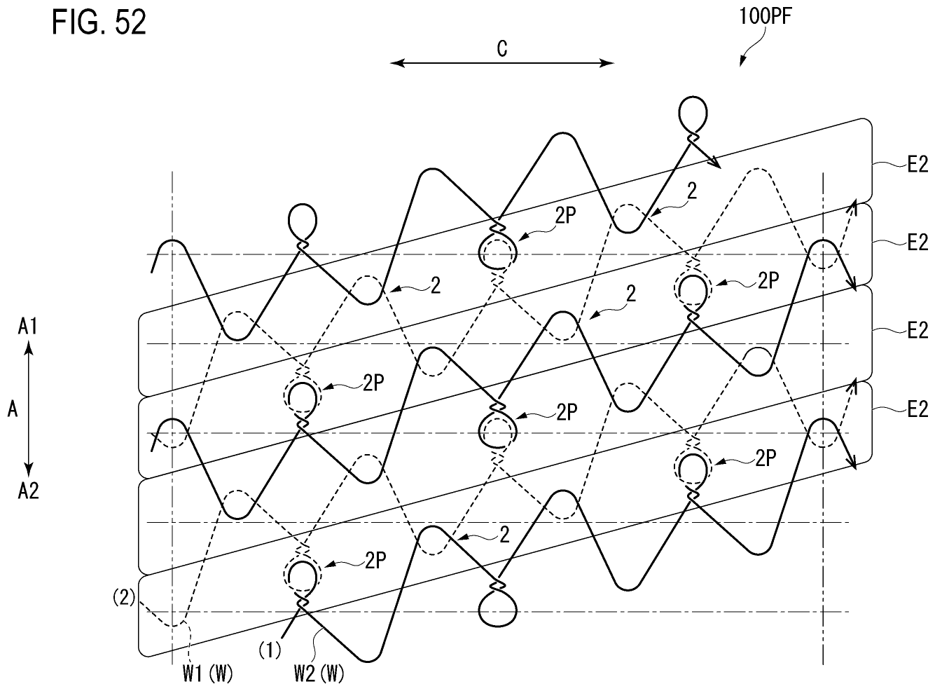

FIG. 52 is a diagram showing how to knit the stent 100PF.

The operator weaves the second wire W2 along a cylindrical jig with a plurality of projecting pins spirally provided. Next, the operator weaves the first wire W1 along a columnar jig provided with a plurality of projecting pins spirally. The operator may weave the first wire W1 first. It should be noted that illustration of the projecting pin is omitted in FIG. 52. Since the two wires W can be woven independently, the operator can easily weave the stent 100PF.

FIG. 53 is a diagram showing how to knit the end region E3 of the stent 100PF. The end region E3 of the stent 100PF on the second direction A2 side is arranged along the circumferential direction C without the valley-shaped bent portion 4 intersecting the peak-shaped bent portion 3. Moreover, the end region (not shown) of the stent 100PF on the first direction A1 side is arranged along the circumferential direction C without the peak-shaped bent portions 3 intersecting the valley-shaped bent portions 4.

The end portions on the second direction A2 side of the valley-shaped bent portions 4 in the end region E3 are aligned with respect to the longitudinal axis direction A as shown in FIG. 53. By aligning the ends, it becomes easy to measure the length of the stent PF in the longitudinal direction A, and it becomes easy to position the stent 100PF when the stent 100PF is placed.

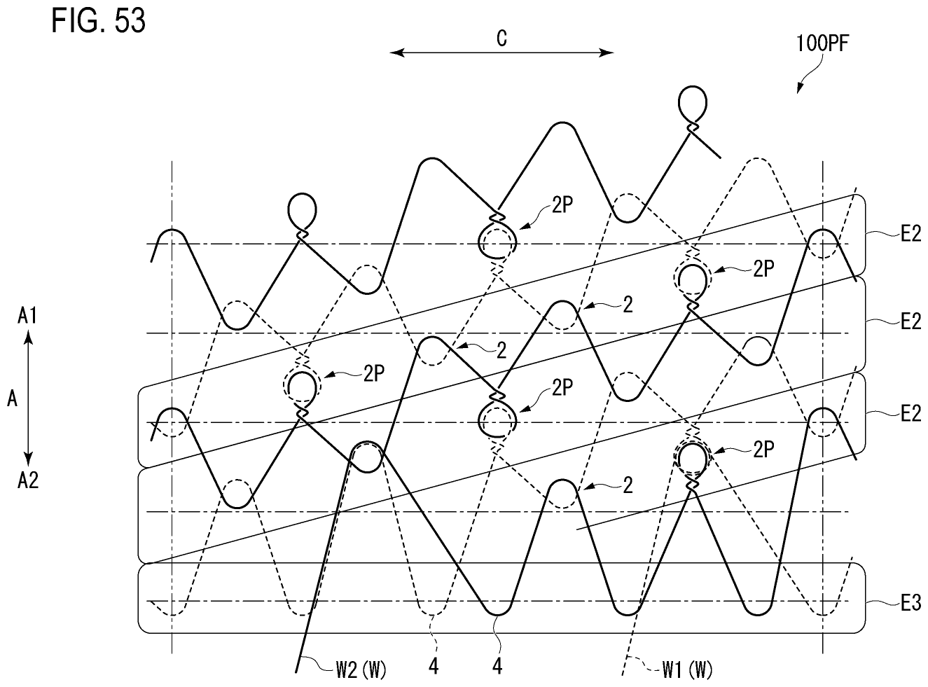

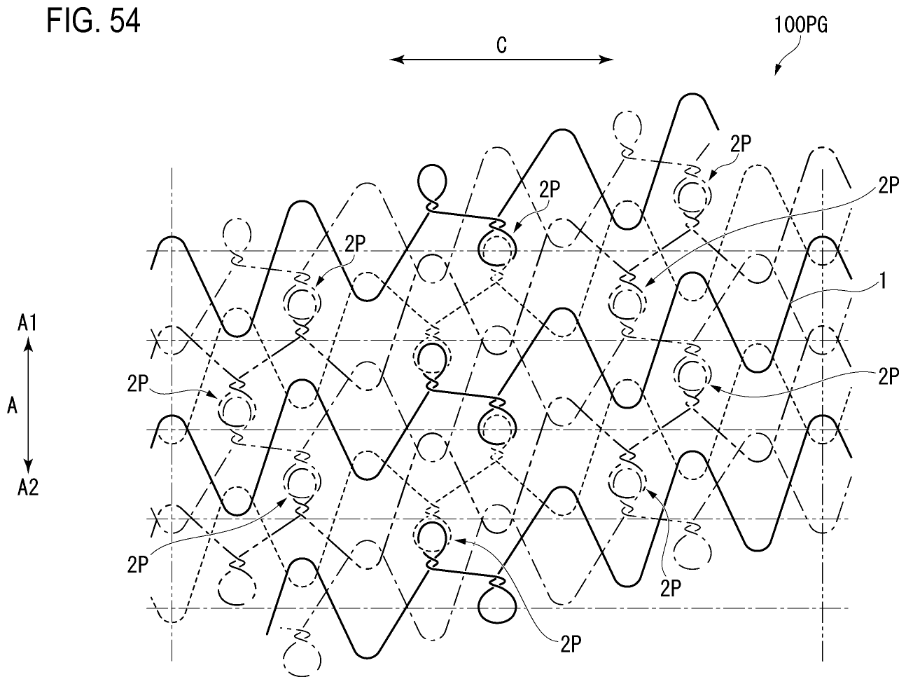

FIG. 54 is an exploded view of a stent 100PG that is a modification of the stent 100 of the first embodiment. A stent 100PG is a stent obtained by replacing part of the interlocking portion 2 of the stent 100 of the first embodiment with a loop interlocking portion 2P. When the stent 100PG is bent as a whole, the stent 100PG maintains its shape due to the friction of the wire W caused by the vertical relationship between the straight-line crossing portions 1 and the interlocking portions 2. The occurrence of crushing in the direction A (axial crushing) can be suppressed.

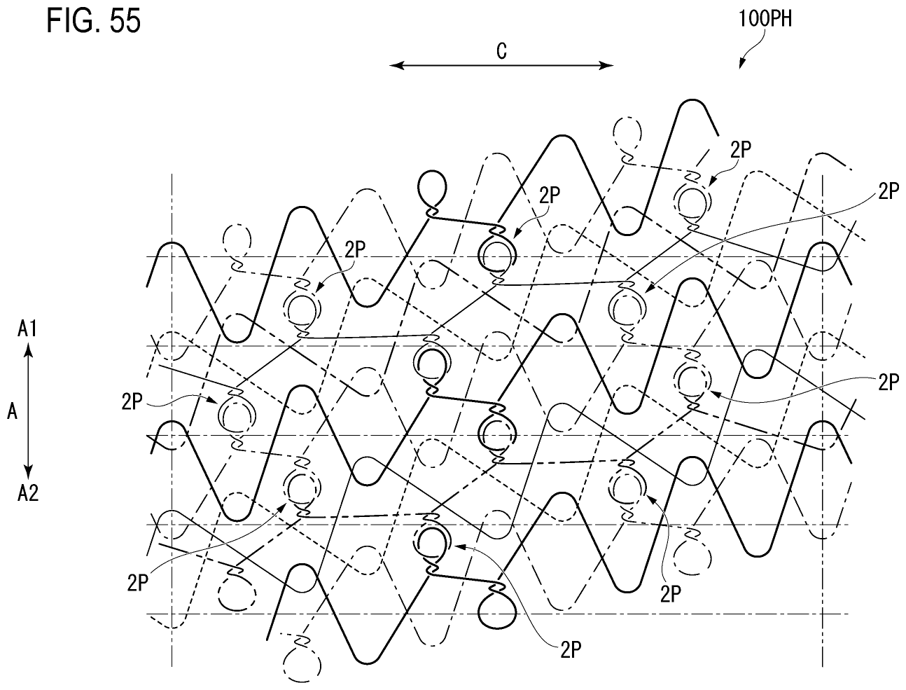

FIG. 55 is an exploded view of a stent 100PH that is a modification of the stent 100I of the ninth embodiment. A stent 100PH is a stent obtained by replacing part of the interlocking portion 2 of the stent 100I of the ninth embodiment with a loop interlocking portion 2P. When the entire stent 100PH is bent, the stent 100PH maintains its shape due to the friction of the wire W caused by the vertical relationship between the straight-line crossing portions 1 and the interlocking portions 2. The occurrence of crushing in the direction A (axial crushing) can be suppressed. Furthermore, the stent 100PH can be adjusted in shape retention, expandability, cell size, etc. by changing the weaving method of the wires W in the first region E1 (the number of overlapping wires W forming the first region E1).

It is desirable that the loop interlocking portions 2P are not arranged adjacent to each other in the second region E2. When the loop interlocking portions 2P are arranged adjacent to each other in the second region E2, a region in which it is difficult to partially move in the longitudinal direction A continuously occurs in the circumferential direction C, so that it is difficult to repel bending (low axial force) is lost. This is because one loop interlocking portion 2P has a high degree of rotational freedom, but two loop interlocking portions 2P continuous in the circumferential direction C have a low degree of rotational freedom.

The loop interlocking portion 2P has enhanced resistance to crushing (axial crushing) in the longitudinal direction A, by being connected to both ends of the straight-line portion 10 via the straight-line portion 10, like the stent 100PB shown in FIG. 47 etc. Preferably, the loop interlocking portions 2P are not arranged adjacent to each other in the second region E2 and are connected via the straight-line portion 10 as described above.

Here, the arrangement mode of the loop interlocking portions 2P in which the loop interlocking portions 2P are not arranged adjacent to each other in all the second regions E2 is called “first arrangement mode”. Further, as shown in FIGS. 47 to 51, the arrangement mode of the loop interlocking portions 2P in which at least one loop interlocking portion 2P is arranged in all the second regions E2 is referred to as a “second arrangement mode”.

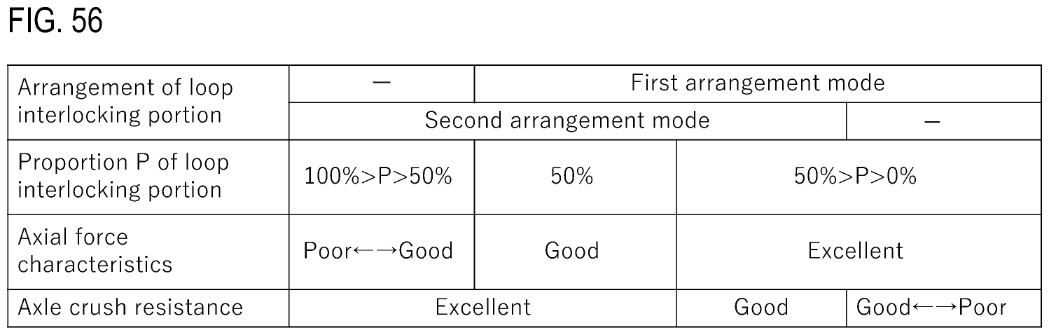

FIG. 56 is a diagram showing the difference in stent properties based on the first arrangement mode and the second arrangement mode.

The ratio P is the ratio (%) of the number of loop interlocking portions 2P to the total number of interlocking portions 2 and loop interlocking portions 2P of the stent P. In calculating the ratio P, only all the second regions E2 need to be considered, and the end regions need not be considered. Axial force characteristics indicate that the material is highly resistant to bending (low axial force). The axial crushing resistance property indicates that the resistance to crushing (axial crushing) in the longitudinal axis direction A is high.

When the arrangement mode of the loop interlocking portion 2P is the first arrangement mode, the axial force characteristics are high (excellent or good) because the degree of freedom of rotation is higher than when the arrangement mode is not the first arrangement mode. When the arrangement mode of the loop interlocking portion 2P is the second arrangement mode, at least one loop interlocking portion 2P having a resistance to axial crushing is arranged in any second region E2, so that the axial crushing resistance characteristic is high (excellent or good). In order for the stent 100P to have both the axial force characteristic and the axial crushing resistance characteristic, it is desirable that the loop interlocking portions 2P are arranged in the first arrangement mode and the second arrangement mode. In that case, the ratio P is desirably 50% or less. This is because if the ratio P exceeds 50%, the axial force characteristics are greatly impaired.

The present invention can be applied to stents formed by weaving wires or the like.

The present invention relates generally to stent devices and, in particular, to a stent device having stent wires interlocking with each other so as to prevent axial shortening and gain flexibility of the stent device at an ideal proportion, particularly when the stent device is bent. The calculated placement of the interlockings of the stent wires provide variations of positive effects to the stent and the patient in whom the stent is placed.

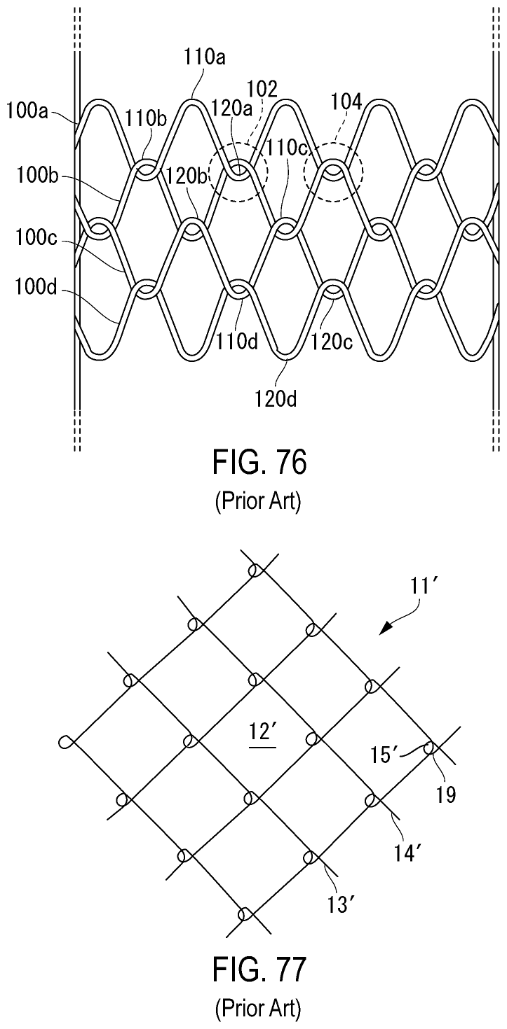

FIG. 76 is a figure of a stent device disclosed in the related art (U.S. Patent Application Publication No. 2013/0226282A1). FIG. 76 discloses the stent wires 100a, 100b, 100c, and 100d each having peaks 110a, 110b, 110c, and 110d and valleys 120a, 120b, 120c, and 120d. The peaks and valleys are occasionally “caught” by each other, for instance the peak 110b of the second stent wire 100b is caught by the valley 120a of the first stent wire 100a at location shown at the reference sign 102 in FIG. 76. On the other hand, the peaks and valleys are occasionally “uncaught” by each other, for instance the peak 110b of the second stent wire 100b is uncaught by the valley 120a of the first stent wire 100a at a location shown at the reference sign 104 in FIG. 76. According to the related art, it is preferred that the number of the peaks 110a, 110b, 110c, and 110d and the number of the valleys 120a, 120b, 120c, and 120d be set to multiples of 3, such that the peaks 110a, 110b, 110c, and 110d caught by the valleys 120a, 120b, 120c, and 120d and the peaks 110a, 110b, 110c, and 110d uncaught by the valleys 120a, 120b, 120c, and 120d are repeated in a ratio of 2:1. It is possible to make the ratio 3:1 or 2:2 instead of 2:1, but there is a risk of damage to connected portions between the stent wires 100 when an external force is applied, since the number of the connected portions between the stents 100a, 100b, 100c, and 100d is decreased.

FIG. 77 is a figure of another stent device disclosed in the related art (U.S. Pat. No. 6,221,100). FIG. 77 discloses a netting 11′ having a mesh pattern where a mesh 12′ is formed with filaments 13′ and 14′ (the term “mesh” or “meshes” refers to the actual cord or wire network, and not the spaces therebetween). The points of an intersection 15′ of the filaments 13′ and 14′ form an eye 19 at every instance, where only one of the filaments is looped around the other filament. The radial bearing strength is increased by the eye 19, although the shortening of the stent by axial compression would not be possible due to the eye 19.

The drawback of the related art stent devices include axial shortening of the stent device occurs due to axial compression after inserting the stent device into the human body. The axial shortening limits the range that the lumen of the stent device can be expanded within the human body. The drawbacks of the related art stent devices also include the inability of an axial shortening and lack of flexibility of the stent device due to the eye formed at every intersection of the stent wires.

Accordingly, there is a need for designing a stent device with an efficient structure in view of the practical usage, which would substantially obviate one or more of the issues due to limitations and disadvantages of related art stent device. An object of the present disclosure is to provide a stent device having an arrangement of looped interlocking regions and non-looped interlocking regions.

Embodiments of the disclosed stent device includes a first stent wire and a second stent wire forming a cylindrical stent body enclosing an interior void space, a primary interlocking structure, and a secondary interlocking structure. The primary interlocking structure includes a first loop formed of the first stent wire and defining a first loop opening and the second stent wire passing through the first loop opening and the secondary interlocking structure includes the first stent wire and the second stent wire passing over each other. The first stent wire includes a first peak and a first valley, and the first loop is located at the first peak or the first valley of the first stent wire.

In embodiments of the disclosed stent device, the secondary interlocking structure may not including a loop.

In embodiments of the disclosed stent device, in the secondary interlocking structure, the first stent wire and the second stent wire may pass over each without forming a loop.

In embodiments of the disclosed stent device, the second stent wire may include a second peak and a second valley, wherein a portion of the first stent wire forming the secondary interlocking structure may be the first peak, wherein a portion of the second stent wire forming the secondary interlocking structure may be the second valley, and wherein, in the secondary interlocking structure, the first peak may be located in the second valley.

Embodiments of the disclosed stent device further comprises the primary interlocking structure including a second loop.

In embodiments of the disclosed stent device, the second loop may be formed of the second stent wire and may define a second loop opening.

In embodiments of the disclosed stent device, a portion of the second stent wire forming the second loop opening may pass through the first loop opening.

In embodiments of the disclosed stent device, the second loop may be formed at a peak or a valley in the second stent wire.

In embodiments of the disclosed stent device, the second loop may be formed of the first stent wire and may define a second loop opening, and wherein the primary interlocking structure may include the second loop formed of the first stent wire.

In embodiments of the disclosed stent device, the first loop and the second loop may be part of a double-loop structure and wherein the first loop may be the most distal of the first loop and the second loop.

In embodiments of the disclosed stent device, the primary interlocking structure may include a third loop.

In embodiments of the disclosed stent device, the third loop may be formed of one of the first stent wire and the second stent wire and may define a third loop opening.

In embodiments of the disclosed stent device, the primary interlocking structure may include a fourth loop.

In embodiments of the disclosed stent device, the fourth loop formed of one of the first stent wire and the second stent wire and may define a fourth loop opening.

In embodiments of the disclosed stent device, the first loop, the second loop, the third loop, and the fourth loop may form two double-loop structures.

In embodiments of the disclosed stent device, a number of the primary interlocking structure may be equal to or less than the number of the secondary interlocking structure.

In embodiments of the disclosed stent device, the first stent wire may not include three consecutive loops along the alternating peaks and valleys.

In embodiments of the disclosed stent device, the first stent wire may include one loop among the four consecutive alternating peaks and valleys.

In embodiments of the disclosed stent device, further comprises the first stent wire may include two loops among the four consecutive alternating peaks and valleys.

Embodiments of the disclosed stent device may further include a stent delivery system including a sheath having a capability to carry the stent device, and a pusher for pushing out the stent device from the sheath.

The specific type of a loop can vary and the non-looped interlocking regions contribute to ease of bending of the stent device and the looped interlocking regions contribute to prevent axial shortening when the stent device is bent. In a longitudinally direction of the stent device parallel to the longitudinal axis, i.e., in the axial direction, the looped interlocking regions can be arranged continuously or non-continuously. In other embodiments, the looped interlocking regions are continuous over two or more, alternatively two to four, sequentially arranged looped interlocking regions. Such improved stent devices have an efficient structure and provide practical administration of the associated medical procedure. At least one or some of the objectives is achieved by the stent device disclosed herein.

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

Additional features and advantages will be set forth in the description that follows, and in part will be apparent from the description, or may be learned by practice of the invention. The objectives and other advantages of the disclosed stent device will be realized and attained by the structure particularly pointed out in the written description and claims thereof, as well as the appended drawings.

The term “patient,” as used herein, comprises any and all organisms and includes the term “subject.” A patient can be a human or an animal.

Other systems, methods, features, and advantages will be, or will become, apparent to one with skill in the art upon examination of the following figures and detailed description. It is intended that all such additional systems, methods, features, and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed stent device as claimed.

The following detailed description of preferred embodiments can be read in connection with the accompanying drawings in which like numerals designate like elements.

Throughout all of the drawings, dimensions of respective constituent elements are appropriately adjusted for clarity. For ease of viewing, in some instances only some of the named features in the figures are labeled with reference numerals.

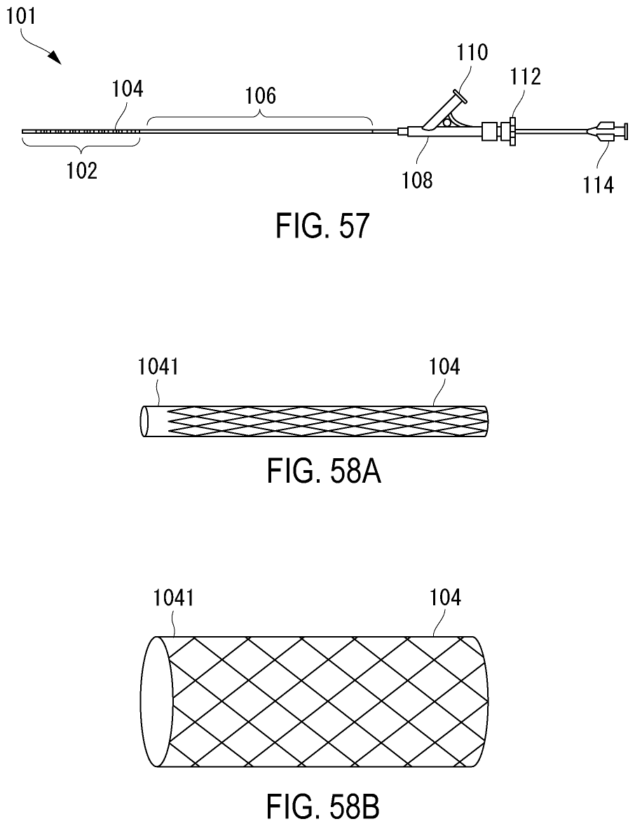

The following detailed description of preferred embodiments can be read in connection with the accompanying drawings in which like numerals designate like elements and in which: FIG. 57 is an illustration of a stent device delivering system 101. The stent delivery system 101 includes a tip portion 102, the stent device 104, a sheath 106, a two port hub 108, a side port 110, a rotatable handle lock 112, and an inner handle 114. The sheath 106 has a two-layered structure with an inner sheath and an outer sheath. The sheath 106 has the stent device 104 in a reduced diameter held between the two layers at the tip portion 102. The tip portion 102 is connected to the inner sheath and the inner handle 114. The outer sheath is connected to the two port hub 108 and the rotatable handle lock 112. After the stent delivery system 101 places the tip portion 102 and the stent device 104 to the desired position, the outer sheath at the tip portion 102 slides toward the proximal side by fixing the inner handle 110 and pulling the rotatable handle lock 112 toward the proximal side of the delivery system 101, the stent device 104 is caused to self-expand from the reduced diameter to the designed diameter. After the outer sheath finishes sliding the entire length of the stent device 104, the delivery system 101 and the stent device 104 are separated, the stent device 104 leaves to be implanted in the patient's body.

FIG. 58A is an illustration of the stent device 104 with the stent body in a contracted state. The extent of the axial shortening occurring as the stent body contracts is dependent on the interlocking structures of the stent wires. The stent device 104 is inserted into the stent delivery system 101 in the contracted state in order for the delivery of the stent device 104 to occur through blood vessels of the patient and other narrow space. As shown in FIG. 58B, after the stent device 104, which is a self-expandable stent, reaches the treating portion and is pushed out from the stent delivery system 101, the stent body self-expands into the size for which the stent was designed for conducting treatment by expanding the treatment portion.

FIG. 59 illustrates a pattern of the stent wires forming the stent body of the stent device 104. The stent wires forms a cylindrical stent body (hereinafter “stent body”). The stent body includes an interior void space. The stent body defines an inner luminal side of the stent body. As disclosed in FIG. 59, the stent wires cross over each other and form cells enclosed by the stent wires, such as a stent cell 302. The interconnection or overlap of the stent wires can be seen in FIG. 59 by, for example, observing the positional relationship of the stent wire 304 as the stent wire 304 intersects with stent wires 306, 308, and 310. The stent wire 304 intersects with the stent wire 306 at an intersection 312, where the stent wire 304 goes under the stent wire 306. The stent wire 304 then intersects with the stent wire 308 at an intersection 314, where the stent wire 304 goes over the stent wire 308. Then the stent wire 304 goes under the stent wire 310 at the next intersection 316. The alternating under and over location of the stent wire 304 with respect to the intersecting wires at each intersection repeats throughout the stent body shown in FIG. 59.

FIG. 60 illustrates another pattern of the stent wires forming the stent body of the stent device 104. As with FIG. 59, the stent wires of the stent device 104 cross over each other and form cells enclosed by the stent wires, such as a stent cell 402. The interconnection or overlap of the stent wires in the stent body shown in FIG. 60 is more complex compared to that in FIG. 59. For example, the stent wires 404 and 406 cross over each other at an intersection 408, but each of the stent wires 404 and 406 bend and form an interlocking structure with a respective further stent wire, such as the stent wires 404 and 410 forming an interlocking intersection 412. Because the stent wires 404 and 410 can move independent of each other, the stent wires 404 and 410 in the region of an interlocking intersection 412 can form an interlocking stent cell 414.

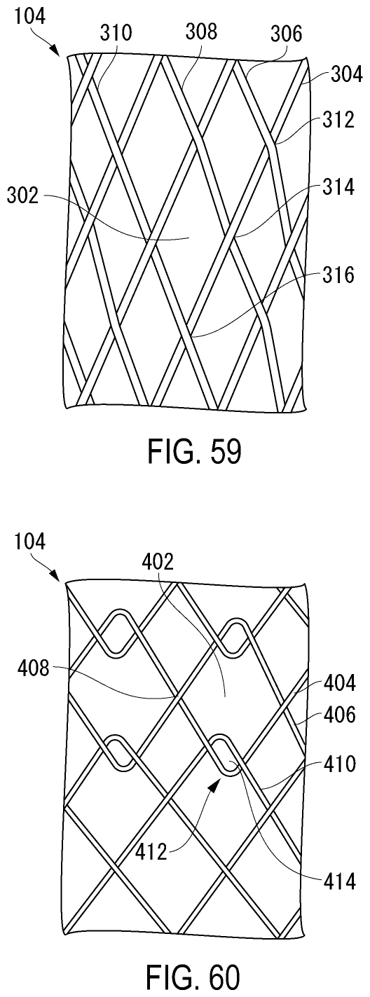

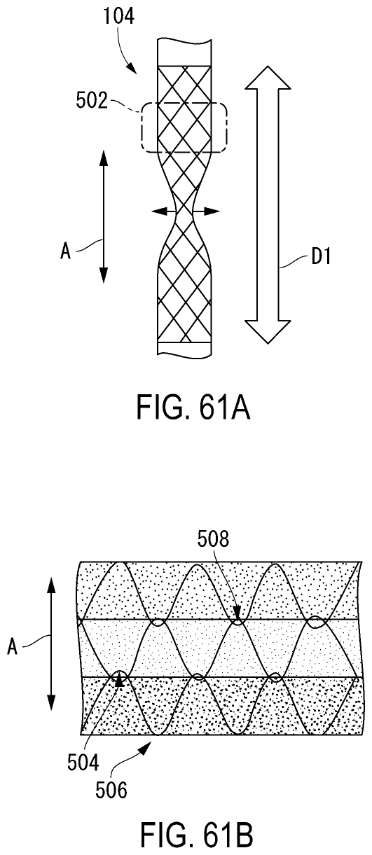

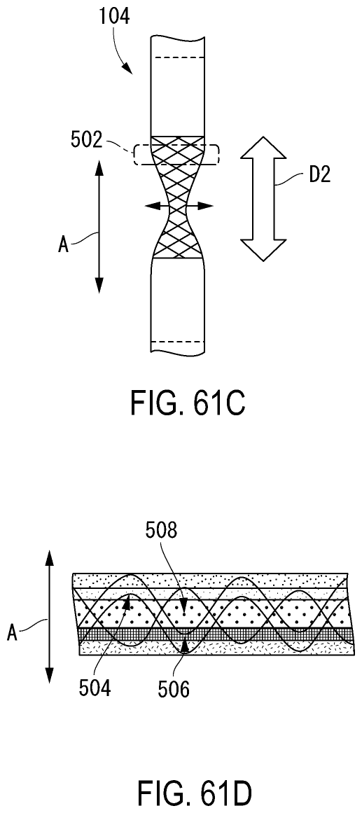

FIGS. 61A to 61D illustrate the mechanism of the axial shortening occurring in the stent device 104. FIG. 61B shows the magnified view of the area 502 in FIG. 61A and illustrates the stent wires of the stent body of the stent device 104 in which the axial shortening has not occurred. In FIG. 61B, each stent wire is separated in the axial direction (represented by arrow A shown in FIG. 61A) with the interlocking portions maximally separated in the axial direction with peaks 504 and valleys 506 of adjacent stent wires intersecting with each other. Depending on the similarity in structure of the adjacent stent wires, the locations of an intersection 508 can be evenly distributed. FIG. 61D shows the magnified view of the area 502 in FIG. 61C and illustrates an example of axial shortening. In FIG. 61D, the stent wires are more closely packed with each other with the peaks 504 and the valleys 506 of the adjacent stent wires no longer forming locations of the intersection 508 and, because the stent wires have moved toward an overlapping arrangement, the stent wires are intermingled with each other. The axial shortening is apparent by comparing distance D1 in FIG. 61A with distance D2 in FIG. 61C.

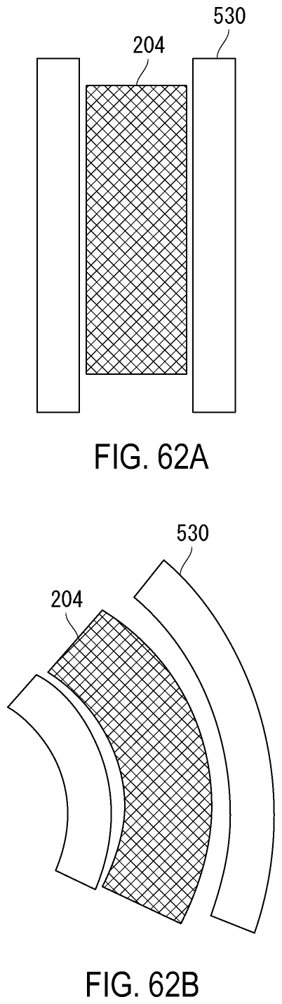

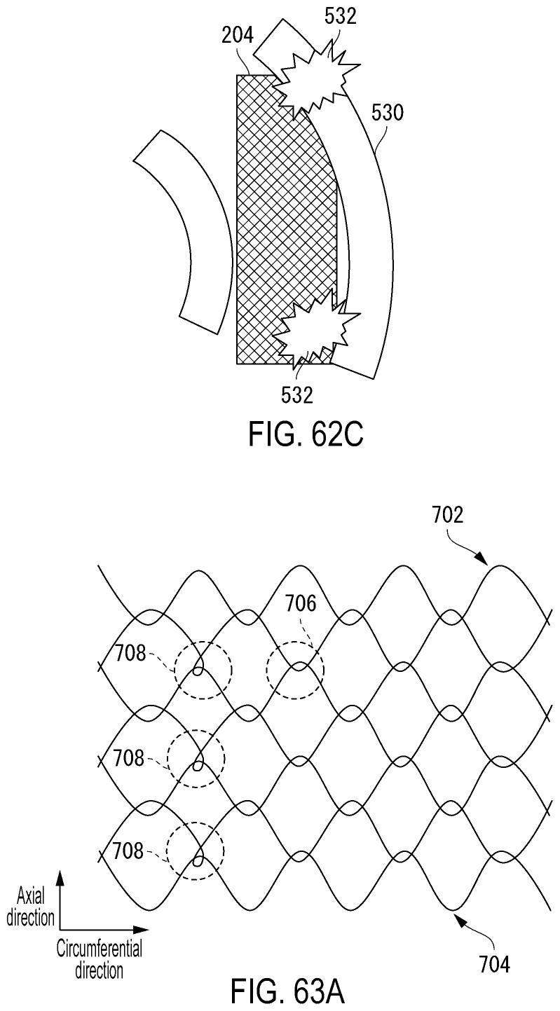

FIGS. 62A to 62C illustrate benefits of flexibility of the stent device 204 after insertion into the patient's body. FIG. 62A discloses the stent device 204 inserted into a treatment portion 530 of a patient. In FIG. 62A, the stent device 204 is in a straight configuration. FIG. 62B discloses the stent device 204 inserted into a treatment portion 530 and in a bent configuration. Due to having adequate flexibility, the stent device 204 is able to bend in accordance with the bending angle of the treatment portion 530. However, as disclosed in FIG. 62C, when the stent device lacks adequate flexibility or even no flexibility would not be able to bend in accordance with the bending angle of the treatment portion 530. Due to this lack of flexibility, it requires an advanced technique for inserting the stent device 204 into the treatment portion 530 for preventing the stent device 204 from contacting with the portion 532 shown in FIG. 62C.

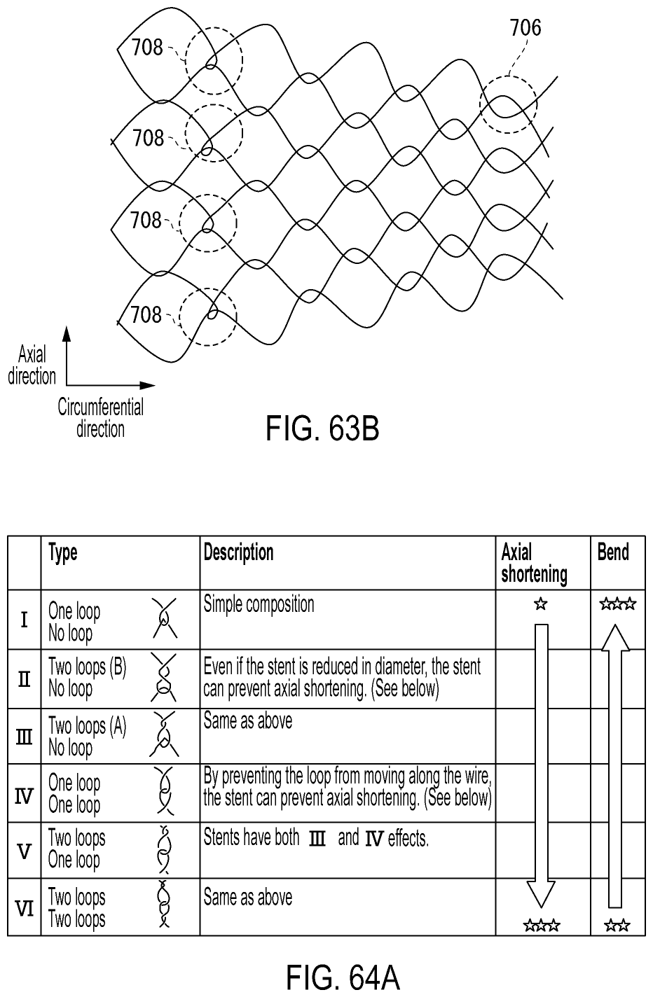

FIGS. 63A and 63B illustrate two types of interlocking structures of stent wires forming a stent body of a stent device. The stent wires typically form peaks and valleys (i.e. the peak 702 and the valley 704). The peaks and valleys in a given stent wire alternately repeats in the circumferential direction of the stent body. A first interlocking type 706 (also called a secondary interlocking structure) is shown in FIGS. 63A and 63B. The first interlocking type 706 is formed by two stent wires interlocking with each other by passing the first stent wire over the second stent wire. In particular, the first interlocking type 706 is characterized by passing the first stent wire over the second stent wire so that when the stent device is expanded in the axial direction, a peak 702 of a first stent wire is located at and passes over a valley 704 of the second stent wire. Because of this arrangement of the first stent wire and the second stent wire in the first interlocking type 706, the peak 702 of one of the stent wires is located in the valley 704 of the other stent wire. A second interlocking type 708 (also called a primary interlocking structure) is also shown in FIGS. 63A and 63B and is formed by two stent wires interlocking with each other by a first stent wire forming a loop over a second stent wire (resulting in the second stent wire passing through the opening formed by the loop in the first stent wire). The first interlocking type 706 may be formed without including a loop.

As illustrated in FIG. 63B, the two stent wires forming the first interlocking type 706 can move relative to each other and loosen, which results in an axial shortening in the case where a force is applied to the stent device in the axial direction. For example, the peak 702 of the first stent wire can move relative to the valley 704 of the other stent wire and the peak 702 become disengaged from the valley 704, e.g., the peak 702 of one of the stent wire can become unhung from the valley 704 of the other stent wire. In contrast and as also illustrated in FIG. 63B, the stent wires forming the second interlocking type 708 are constrained from moving relative to each other by the loop structure in that the stent wires forming the second interlocking type 708 do not become disengaged from each other, and the stent body having second interlocking type 708 structures do not exhibit axial shortening even in the case where a force is applied to the stent device in the axial direction. However, even though the stent wires forming the second interlocking type 708 do not become disengaged from each other, the first stent wire passing through the loop structure of the first stent wire can, in some embodiments, move such that the first stent wire moves in the circumferentially direction relative to the stent wire with the loop structure. Additionally, to the extent the first stent wire has a construction with some axial positional variation (such as a pattern of peaks and valleys), the two stent wires can, in some embodiments, also have relative motion in the axial direction, but such axial motion will be constrained by the distance from a peak to a valley on the respective first stent wire passing through the loop structure. Combinations of a relative circumferential movement and a relative axial movement may also occur in the stent devices incorporating the second interlocking type 708.

Generally speaking, the stent device may be made by multiple stent wires or from a single stent wire. The interlocking structures may seem to require more than one stent wires to intertwine with each other, but a single stent wire may be used to construct the entire cylindrical stent structure through forming various interlocking structures by the single stent wire.

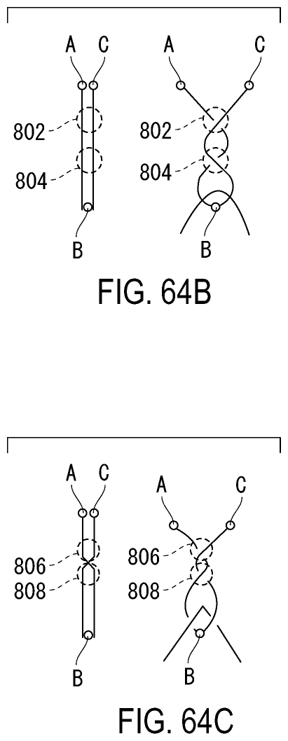

FIG. 64A is a chart disclosing the six exemplary types of interlocking portions that can be used to form the second interlocking type 708 structures of the stent body. The various exemplary types of second interlocking type 708 structures each includes at least one loop using the stent wires. In some embodiments, the second interlocking type 708 structure includes one loop on only first stent wire, and the other stent wire has no loop (i.e., is non-looped), in other embodiments, the second interlocking type 708 structure includes combinations of one or more loops on one of the two stent wires and none or one or more loops on the other of the two stent wires.

The following description of six exemplary types of interlocking portions is made with reference to FIGS. 64A to 64C. Second interlocking type No. I is formed by first stent wire forming a single loop and the other stent wire not forming a loop. Second interlocking type No. I is also shown in FIGS. 63A and 63B. Second interlocking type No. II is formed by first stent wire forming a double-loop and the other stent wire not forming a loop. The stent wire without the loop can pass through the opening of either one or the other of the double-loop, although typically, the stent wire without the loop will pass through the distal of the two loops (as shown in FIG. 64A). FIG. 64B discloses the schematic view of Second interlocking type No. II. A double-loop is formed between A and C, forming two intersections 802 and 804. Here, A to B is the “outward wire” and B to C is the “inward wire”. In FIG. 64B, the outward wire passes under the inward wire at both intersection 802 and 804. Second interlocking type No. III is similar to Second interlocking type No. II. FIG. 64C discloses the schematic view of Second interlocking type No. III. A double-loop is formed between A and C, forming two intersections 806 and 808. Here, A to B is the “outward wire” and B to C is the “inward wire”. In FIG. 64C, the outward wire passes under the inward wire at intersection 802 and outward wire passes over the inward wire at 804. In other words, in Second interlocking type No. III, the stent wire that is on the top at the intersection 806 is different from the stent wire that is on top at the intersection point 808. Thus, unlike Second interlocking type No. II, the outward and inward wires are twisted in Second interlocking type No. III, making the double-loop for Second interlocking type No. III tighter than Second interlocking type No. II. The stent wire without the loop can pass through the opening of either one or the other of the double-loop, although typically, the stent wire without the loop will pass through the distal of the two loops (as shown in FIG. 64A). Second interlocking type No. IV is formed by both stent wires forming a single loop. The two single loops are interconnected with each other as shown in FIG. 64A. Second interlocking type No. V is formed by first stent wire forming a double-loop and the other stent wire forming a single loop. The single loop is interconnected with double-loop by the wire of the single loop passing through one of the openings of either one or the other of the double-loop, although typically, the single loop will pass through the distal of the two loops (as shown in FIG. 64A). Finally, second interlocking type No. VI is formed by both stent wires forming a double-loop. The loop of the first stent wire is interconnected with a loop of the second stent wire, although typically, the distal loops of both double-loop structures will pass through each other (as shown in FIG. 64A). As disclosed in FIG. 64C, the intertwining of the outward and inward wires may be made more complex structure (i.e. twisted) in order to make the double-loop structure for Second interlocking types No. V and No. VI tighter as compared to the structure disclosed in FIG. 64B.

Depending on the combination of non-loop, single loop, and double-loop on the two stent wires, the axial shortening and flexibility (bending) can vary. For example, and as shown in FIG. 64A, the level of axial shortening increases from second interlocking type No. I to No. VI and the level of flexibility of the stent device increases from second interlocking type No. VI to No. I, due to the number of loops involved.

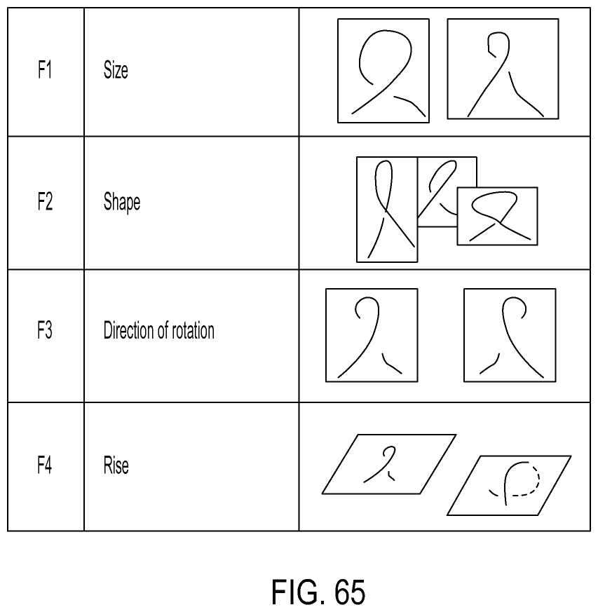

FIG. 65 is a chart disclosing the four factors (factors F1 to F4) that may affect the functions of each loops included in the various second interlocking types. Factor F1 is the size of the loop. Increasing the size of the loop increases the degree of freedom of the stent wires at the interlocking portions and improves the overall flexibility of the stent device. Factor F2 is the shape of the loop. The asymmetrical shape of the loop may create unevenness in the flexibility of the stent wires forming the loop, resulting in unevenness in the flexibility of the interlocking portion. Factor F3 is direction of the loop rotation. By changing the direction of the rotation of the loop, it is possible to adjust the direction of the increased flexibility and decreased flexibility of the stent wires constituting the interlocking portion. Factor F4 is the rise of the loop. The raised structure of the loop makes the surface of the stent device uneven, which may minimize or prevent migration of the stent device within the treatment portion of the patient.

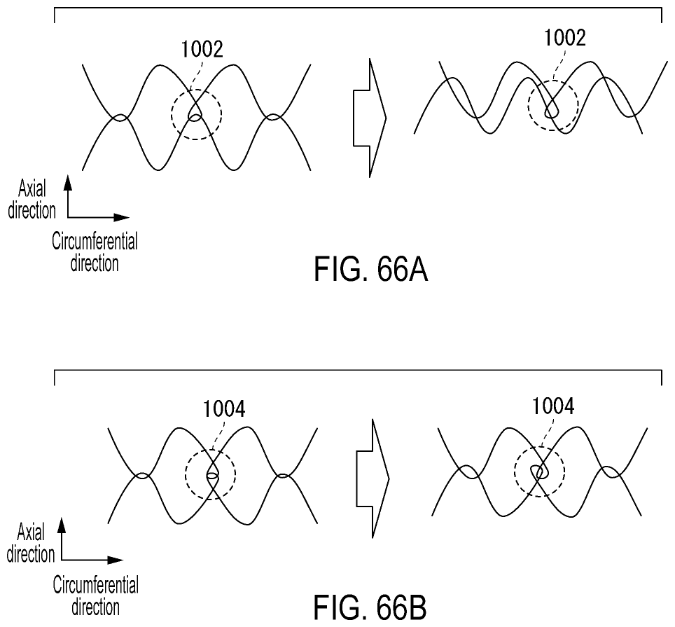

FIGS. 66A and 66B illustrate how forming loops in both stent wires (i.e. second interlocking type No. IV, No. V, and No. VI) to prevent an axial shortening of the stent device. FIG. 66A shows two stent wires forming a single Second interlocking portion type No. I. In case a force is applied to the stent device in the axial direction, the loop 1002 in the Second interlocking portion type No. I slides along the other stent wire, e.g., movement in the circumferential direction, reducing the distance (in the axial direction) between stent wires and allowing axial shortening of the stent device to occur. In contrast, as shown in FIG. 66B, the two stent wires forming a single Second interlocking portion type No. IV with one loop formed on each of the stent wires, the loops 1004 formed on both stent wires prevent the loops 1004 from sliding and contribute to maintaining the distance (in the axial direction) between stent wires and prevent an axial shortening of the stent device from occurring. Second interlocking portions No. V and No. VI, in which both first wire and the second wire form a loop, have the same effect as Second interlocking portion type No. IV.

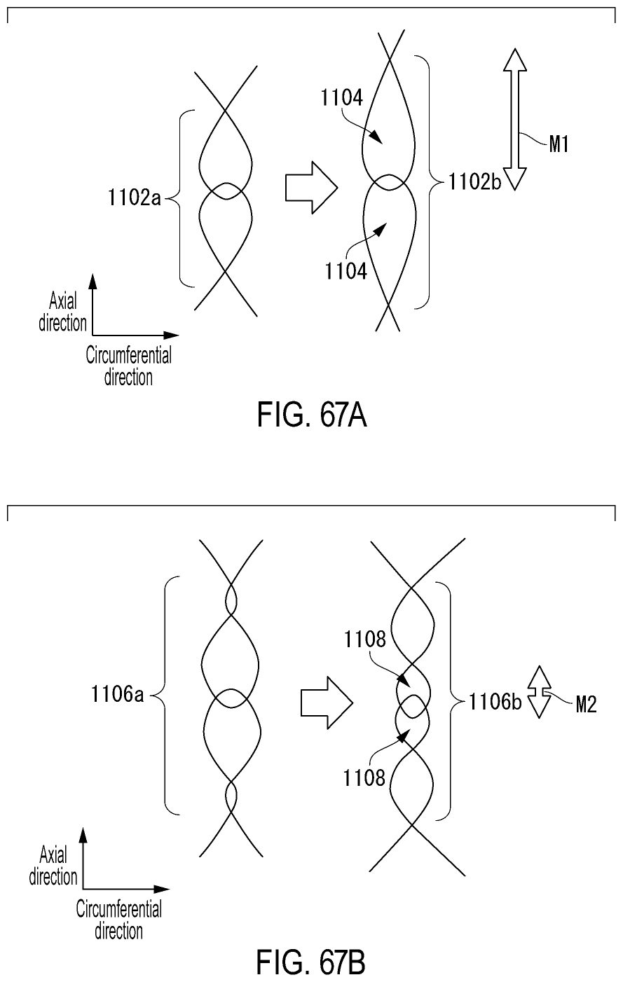

FIGS. 67A and 67B illustrate how forming double-loops in both stent wires (i.e. Second interlocking portion type No. IV) to prevent an axial shortening of the stent device. FIG. 67A discloses two stent wires forming Second interlocking portion type No. IV with one loop formed on each of the stent wires. Through contraction of the stent device, such as is necessary for embedding the stent delivery system as described in FIG. 58A, a force in the circumferential direction is applied to the stent wires. As disclosed in FIG. 67A, after the force applied in the circumferential direction, the shape of the loop is extended in the axial direction (compare distance 1102a to distance 1102b), which can provide more space for an axial shortening to occur in case a force is applied in the axial direction. For example, the open space 1104 within each loop provides freedom of movement in the axial direction to the stent wires, which can move as indicated by arrow M1 shown in FIG. 67A. In contrast, as shown in FIG. 67B, when the two stent wires form Second interlocking portion type No. VI with double-loops formed on both stent wires, the difference in the axial distance between the shape of the loops when a force is applied in the circumferential direction (right side of FIG. 67B) and when a force is not applied in the circumferential direction (left side of FIG. 67B) (compare distance 1106a to distance 1106b) is much less than in the FIG. 67A configuration. Although the overall length in the axial direction is related to the axial length of the double-loop structure, the open space 1108 in the loops that are interconnected provide space for freedom of movement in the axial direction for the stent wires which is limited as indicated by arrow M2 shown in FIG. 67B. As the distance M2 is less than the distance M1, the double-loops in FIG. 67B minimizes and prevents axial shortening in the case a force is applied in the axial direction upon or after the stent device is put in a contracting state to a greater extent than the single loop structure in FIG. 67A.

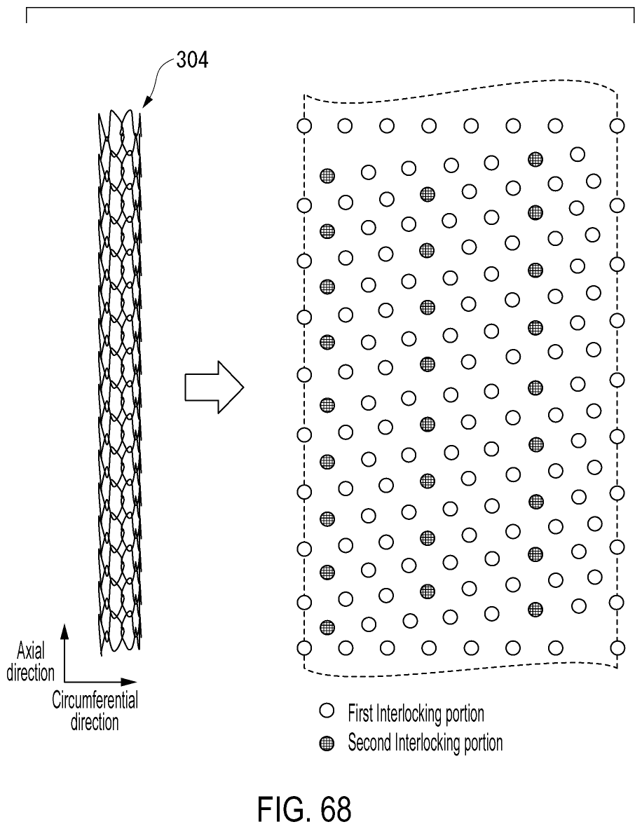

FIG. 68 illustrates the allocation of the two variations of the interlocking portions, the First Interlocking portion and the Second Interlocking portion within the stent device. The First Interlocking portion has connections between the stent wires consistent with the first interlocking portion types disclosed herein and the Second Interlocking portion has connections between the stent wires consistent with the Second interlocking portion types disclosed herein. The schematic on the right shows the location of the two different interlocking portions of the cylindrical stent device, where the cylindrical stent device has been illustrated as a sheet-like structure. The open dot represents the First Interlocking portion, e.g., the structure having no loop within the interlock structure, and the filled dot represents the Second Interlocking portion, e.g., the structure having at least one loop within the interlock structure. In the FIG. 68 embodiment, the Second Interlocking portions are linearly located in the axial direction.

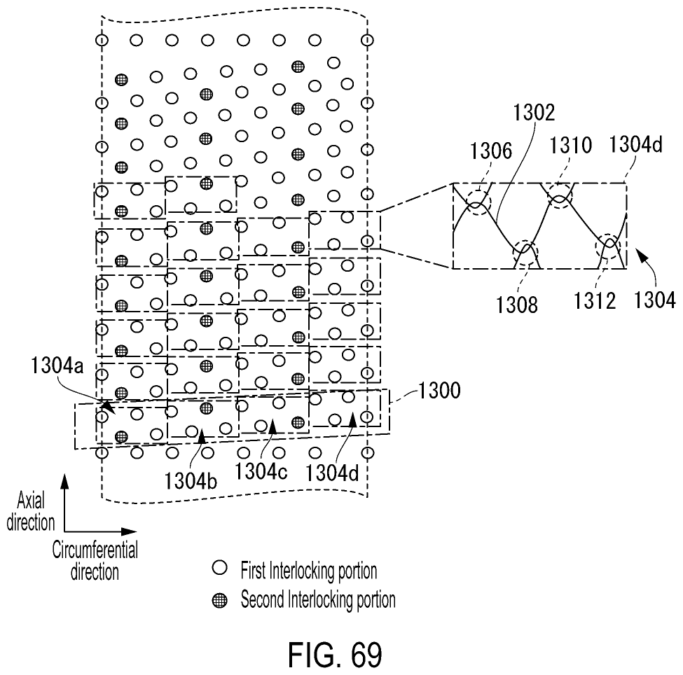

FIG. 69 illustrates the relationship of the four neighboring interlocking portions (in the circumferential direction) sharing a single stent wire 1302, forming an interlocking block 1304. As disclosed in FIG. 69, an interlocking block requires a stent wire to interlock with the two adjacent stent wires forming four consecutive interlocking portions. There is a plurality of such interlocking blocks 1304 that extend across the length of the stent device in the circumferential direction (for example, the four interlocking blocks 1304 in area 1300 shown in FIG. 69 forming a row of the interlocking blocks 1304). A plurality of such rows of the interlocking blocks 1304 extend in the axial direction. Within each row of the interlocking blocks 1304, the arrangement of the four neighboring interlocking portions contained within one interlocking block 1304 varies. For example, each of the interlocking block 1304a, 1304b, and 1304c in area 1300 has one Second Interlocking portion and three First Interlocking portion (as indicated by the open dot (representing First Interlocking portion) and the filled dot (representing the Second Interlocking portion)) and the interlocking block 1304d within the area 1300 has no Second Interlocking portion. A magnified view of the interlocking block 1304d is shown in FIG. 69. In the magnified view of the interlocking block 1304d, the single stent wire 1302 interlocks with the other two stent wires at the interlocking portions 1306, 1308, 1310, and 1312, each of the four interlocking portions being a First Interlocking portion.

As shown in FIG. 69, the location of the Second Interlocking portion among the four neighboring interlocking portions in any one interlocking block 1304 changes within the stent device as a function of a location in the circumferential direction. Thus, in the interlocking block 1304a, the Second Interlocking portion is the second location; in the interlocking block 1304b, the Second Interlocking portion is the third location; and in the interlocking block 1304c, the Second Interlocking portion is the fourth location. The stent wires are interconnected in a repeated pattern. The first two consecutive peaks of the first stent wire are caught by the first two consecutive valleys of the adjacent stent wire and then one subsequent peak of the first stent wire is uncaught by one subsequent valley of the adjacent stent wire. The first stent wire may be formed without including three consecutive loops along the alternating peaks and valleys.

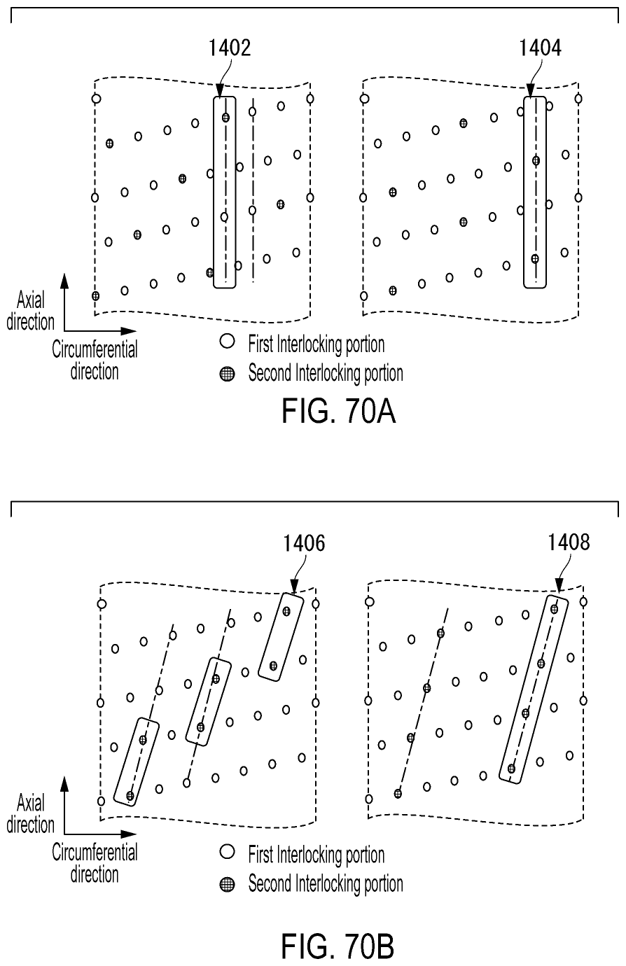

FIG. 70A is a schematic representation of a stent body showing alternate allocations (numbers and locations) of the First Interlocking portions and the Second Interlocking portions. Interlocking block 1402 discloses two interlocking portions aligned in the axial direction where one of the interlocking portions is a First Interlocking portion and the other is a Second Interlocking portion. Interlocking block 1404 discloses two interlocking portions aligned in the axial direction where both of the interlocking portion is the Second Interlocking portion. Interlocking block 1404, two or more of the Second Interlocking portions are placed in the same line in the axial direction. This configuration can prevent axial shortening of the stent device better than Interlocking block 1402. The Second Interlocking portions do not necessarily need to align in a continuous manner. A plurality of the Second Interlocking portions arranged in the same axial direction contributes to preventing axial shortening of the stent device.

FIG. 70B is another schematic representation of a stent body showing alternate allocations (numbers and locations) of the First Interlocking portions and the Second Interlocking portions. The Interlocking block 1406 discloses two consecutive Second Interlocking portions aligned in the inclined direction. In other words, in the Interlocking block 1406, the Second Interlocking portions are formed on the continuous peak and valley of the stent wire. The structure of the interlocking bloc 1406 prevents an axial shortening of the stent device more than the Interlocking block 1402. Interlocking block 1408 discloses four consecutive Second Interlocking portions aligned in the inclined. This configuration in the Interlocking block 1408 can prevent an axial shortening of the stent device better than the Interlocking block 1406. Placing more the Second Interlocking portions in the inclined direction than the Interlocking block 1406 will contribute to preventing an axial shortening of the stent device, but the number of the Second Interlocking portions need not be four.

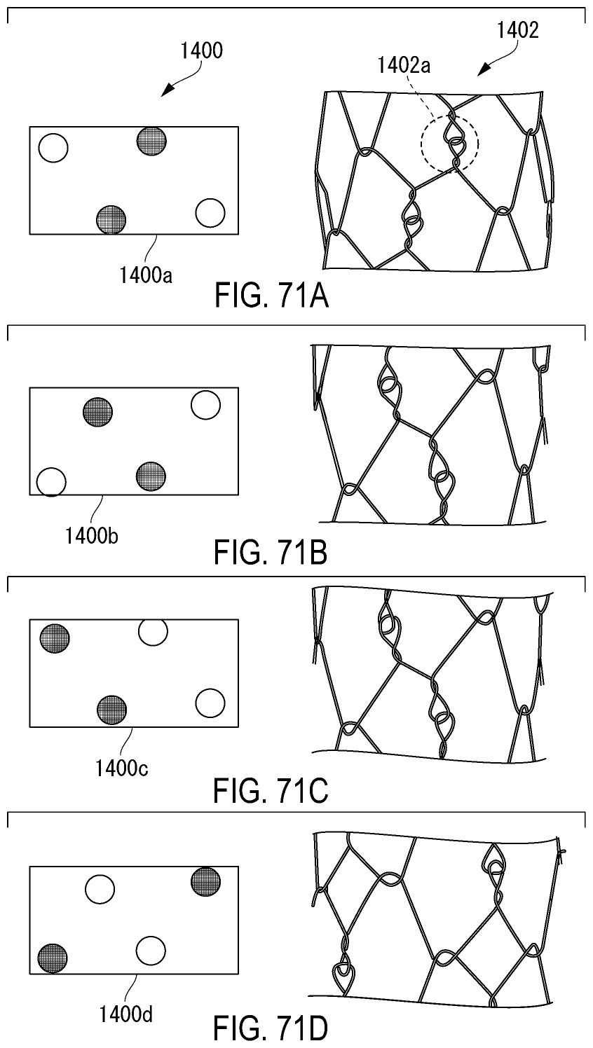

FIGS. 71A to 71D illustrate various interlocking blocks 1400 relative to the actual implementation 1402 in the stent device. FIGS. 71A to 71D disclose interlocking blocks 1400a, 1400b, 1400c, and 1400d, each of which include two First Interlocking portions (i.e. including no loop) (indicated by open dot) and two Second Interlocking portions (i.e. including at least one loop) (indicated by filled dot). FIGS. 71A to 71D disclose the Second Interlocking portion 1402a in the form of the second interlocking type No. VI, in which both stent wires forming the interlocking portions include double-loops.

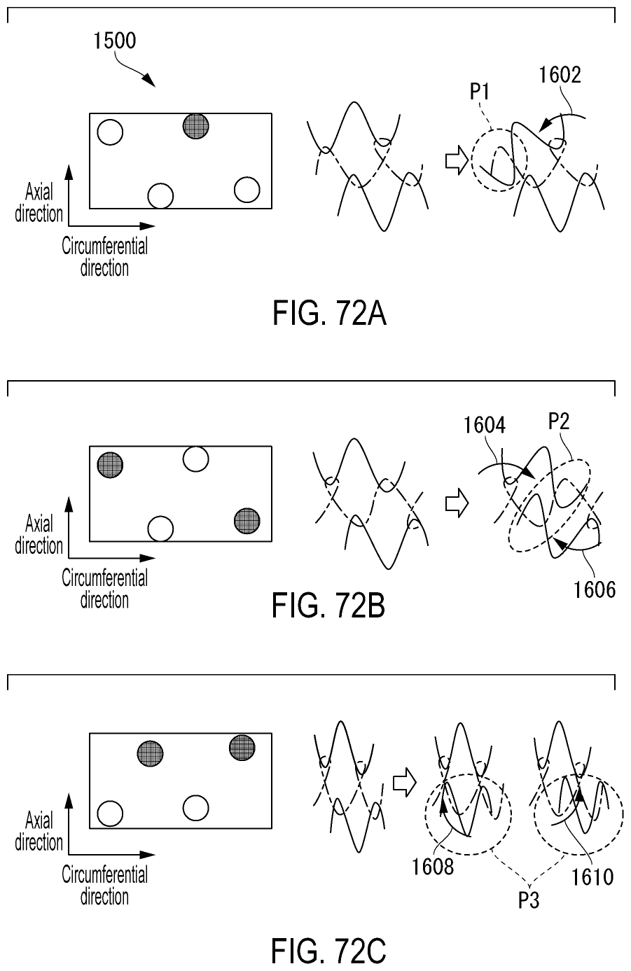

FIGS. 72A to 72C illustrate the relationship between the allocation, e.g., number and location, of the Second Interlocking portions within an interlocking block 1500 and the effects of such different allocations on the flexibility of the stent device. FIG. 72A discloses an interlocking block with one Second Interlocking portion (the second interlocking type No. I) (represented by filled dots) and three First Interlocking portions (the first interlocking type, i.e. with no loop) (represented by open dots). The line schematics illustrate the relationships of the stent wires and how the Second Interlocking portion interacts with the First Interlocking portions. The line schematics show that when a force is applied to the stent device in the circumferential direction (arrow 1602 shown in FIG. 72A), the loosely interlocked the First Interlocking portions provide leeway to the stent wires to intermingle, resulting in flexibility of the stent device (see e.g., the region P1 where the stent wires in the First Interlocking portion have moved apart and separated). However, the Second Interlocking portions maintain their interlocking relationship.

FIGS. 72B and 16C disclose other examples of interlocking blocks with two Second Interlocking portions (the second interlocking type No. I) (represented by filled dots) and two First Interlocking portions (the first interlocking type, i.e. with no loop) (represented by open dots). The line schematics also show that when a force is applied to the stent device in the circumferential direction (arrows 1604, 1606, 1608, and 1610 shown in FIGS. 72B and 72C), the loosely interlocked the First Interlocking portions provide leeway to the stent wires to intermingle, resulting in flexibility of the stent device (see e.g., regions P2 and P3, where the stent wires in the First Interlocking portions have moved apart and separated). However, the Second Interlocking portions maintain their interlocking relationship.

The ratio of a number of the Second Interlocking portions against the First Interlocking portions is ideally 1:3 or 2:2, or alternatively 0.15 to 0.60, 0.15 to 0.40, 0.15 to 0.30, 0.25 to 0.40, or 0.40 to 0.60.

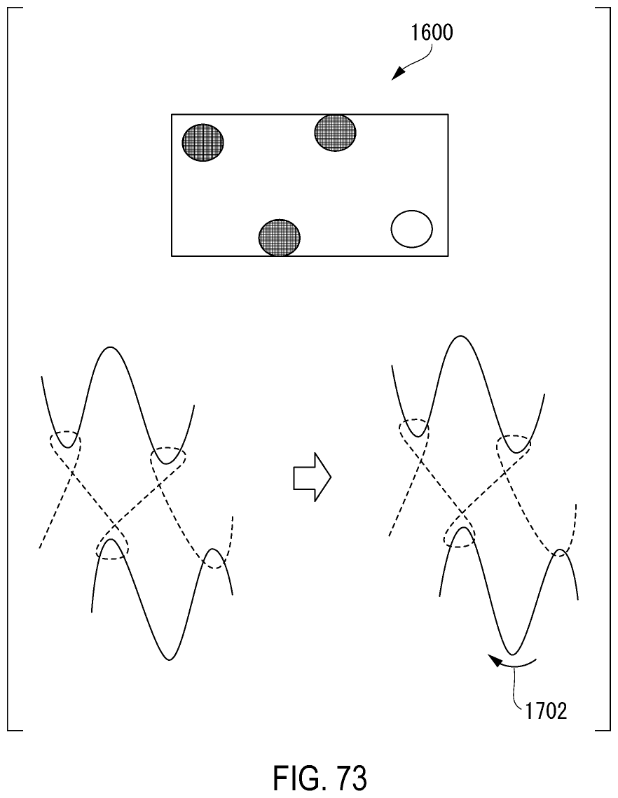

FIG. 73 illustrates an interlocking block 1600 with three Second Interlocking portions (the second interlocking type No. I) (represented by filled dots) and one First Interlocking portion (the first interlocking type, i.e. with no loop) (represented by open dots). The line schematics show that, in this embodiment, when a force is applied to the stent device in the circumferential direction (arrow 1702 shown in FIG. 73), the loosely interlocked the First Interlocking portion does not provide leeway to the stent wires to intermingle, resulting in no flexibility of the stent device. This lack of flexibility is generally seen in the interlocking blocks including three or more Second Interlocking portions.

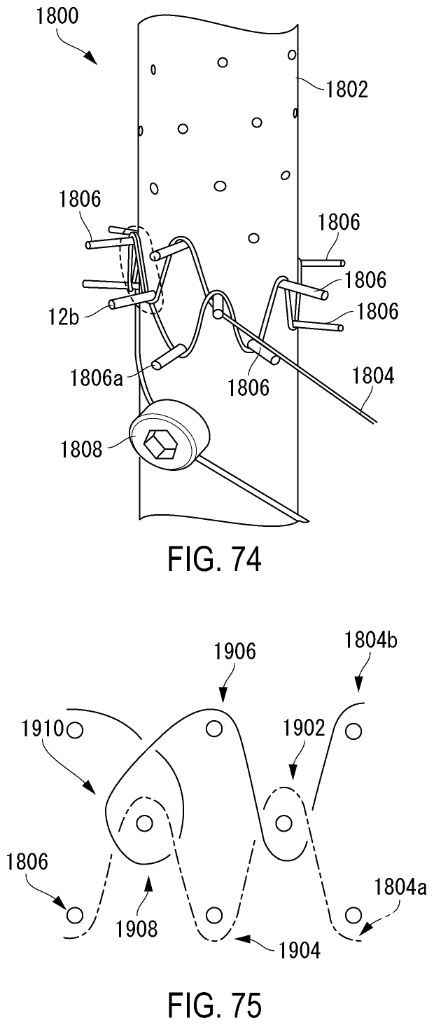

FIG. 74 illustrates an example of a method for manufacturing of the stent device. The method of manufacturing a stent device includes a process of preparing a jig 1800 having a cylindrical shaft 1802, and a braiding process of winding at least one stent wire 1804 from a proximal end of the shaft 1802 to a distal end of the shaft 1802 in a spiral around a longitudinal axis of the shaft 1802. The jig 1800 has a plurality of pins 1806 that are attached to the outer periphery of the shaft 1802, and holes are formed at transition points on the outer periphery of the shaft 1802 for inserting the pins 1806. The holes in the shaft 1802 correspond to the open dots of the First Interlocking portion and filled dots of the Second Interlocking portion illustrated in FIG. 68. The holes in the shaft 1802 are located at the intersection of a plurality of the circumferential dividing lines that extend in the longitudinal direction of the shaft 1802 and equally divide the circumference of the shaft 1802 into a plurality of pieces, and a plurality of the length dividing lines that extend in the circumferential direction of the shaft 1802 and equally divide the length of the shaft 1802 into a plurality of portions. In the process of preparing the jig 1800, a pin 1806 is attached to each hole of the shaft 1802. The multiple pins 1806 attached to the holes are arranged along a spiral path around the longitudinal axis of the shaft 1802.

During the braiding process, one end of the stent wire 1804 is secured to an anchor pin 1808, and the stent wire 1804 is extended from the anchor pin 1808 to the starting pin 1806a, which is the nearest pin located on the length division line. The stent wire 1804 is extended in the circumferential direction of the shaft 1802 from the starting pin 1806a and wound in a zigzag manner around the longitudinal axis of the shaft 1802. The process forms a plurality of wound stent wires 1804. At this time, the stent wire 1804 is extended in a zigzag manner in the circumferential direction while alternately passing through the pins 1806 on one length division line and the pins 1806 on other length division lines adjacent to the distal side of one length division line. This forms the peak on the pin 1806 on one length division line and the valley on the pin 1806 on the other length division line.

FIG. 75 further illustrates an example of a method for manufacturing of the stent device. As described in FIG. 75, the stent wire 1804a extends in the circumferential direction while alternately passing through the pins 1806 in a zigzag manner forming peak 1902 and valley 1904. The stent wire 1804b also extends in the circumferential direction while alternately passing through the pins 1806 in a zigzag manner forming peak 1906 and valley 1908. The stent wire 1804b may form a loop around the pins 1806 and interlock with the stent wire 1804a, forming a loop 1910 at the second valley 1908 of the stent wire 1804b, which may be a single loop or multiple loops, forming the various types of interlocking portions described in the aforementioned embodiments.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

Embodiments of the disclosed stent device further comprises a ratio of a number of the primary interlocking structure to a number of the secondary interlocking structure being 0.15 to 0.60, alternatively 0.15 to 0.40 or 0.15 to 0.30 or 0.25 to 0.40 or 0.40 to 0.60.

Embodiments of the disclosed stent device further comprises a stent cover, wherein the stent cover covers at least a portion of an outer circumferential surface of the cylindrical stent body.

Embodiments of the disclosed stent device further comprises a stent cover, wherein the stent cover covers at least a portion of an inner circumferential surface of the cylindrical stent body.

Embodiments of the disclosed stent device further comprises a stent cover, wherein a first portion of the stent cover covers at least a portion of an outer circumferential surface of the cylindrical stent body and wherein a second portion of the stent cover covers at least a portion of an inner circumferential surface of the cylindrical stent body.

In embodiments of the disclosed stent device, the two loops may be consecutively placed along the alternating peaks and valleys.

Embodiments of the disclosed stent device further comprises the two loops being not consecutively placed along the alternating peaks and valleys.

Embodiments of the disclosed stent device further comprises the two loops interlocking with the second stent wire.

Embodiments of the disclosed stent device further comprises the first loop being asymmetric.

Embodiments of the disclosed stent device further comprises the first loop protruding outward as viewed from the inner lumen of the stent device.

Embodiments of the disclosed stent device further comprises the first stent wire and second stent wire being single wires.

The improved stent devices have an efficient structure and provide practical administration of the associated medical procedure.

A first aspect is a stent device including:
a first stent wire and a second stent wire forming a cylindrical stent body, wherein the cylindrical stent body encloses an interior void space;
a primary interlocking structure; and
a secondary interlocking structure,
wherein the primary interlocking structure includes a first loop formed of the first stent wire and defining a first loop opening and the second stent wire passing through the first loop opening,
wherein the secondary interlocking structure includes the first stent wire and the second stent wire passing over each other, and
wherein the first stent wire includes a first peak and a first valley, and the first loop is located at the first peak or the first valley of the first stent wire.

A second aspect is the stent device according to the first aspect, wherein the secondary interlocking structure does not include a loop.

A third aspect is the stent device according to the first aspect, wherein, in the secondary interlocking structure, the first stent wire and the second stent wire pass over each without forming a loop.

A fourth aspect is the stent device according to the second aspect, wherein the second stent wire includes a second peak and a second valley, wherein a portion of the first stent wire forming the secondary interlocking structure is the first peak, wherein a portion of the second stent wire forming the secondary interlocking structure is the second valley, and
wherein, in the secondary interlocking structure, the first peak is located in the second valley.

A fifth aspect is the stent device according to the first aspect, wherein the primary interlocking structure includes a second loop.

A sixth aspect is the stent device according to the fifth aspect, wherein the second loop is formed of the second stent wire and defines a second loop opening.

A seventh aspect is the stent device according to the sixth aspect, wherein a portion of the second stent wire forming the second loop opening passes through the first loop opening.

An eighth aspect is the stent device according to the sixth aspect, wherein the second loop is formed at a peak or a valley in the second stent wire.

A ninth aspect is the stent device according to the fifth aspect, wherein the second loop is formed of the first stent wire and defines a second loop opening, and wherein the primary interlocking structure includes the second loop formed of the first stent wire.

A tenth aspect is the stent device according to the ninth aspect, wherein the first loop and the second loop are part of a double-loop structure, and wherein the first loop is the most distal of the first loop and the second loop.

An eleventh aspect is the stent device according to the fifth aspect, wherein the primary interlocking structure includes a third loop.

An twelfth aspect is the stent device according to the eleventh aspect, wherein the third loop is formed of one of the first stent wire and the second stent wire and defines a third loop opening.

A thirteenth aspect is the stent device according to the eleventh aspect, wherein the primary interlocking structure includes a fourth loop.

A fourteenth aspect is the stent device according to the thirteenth aspect, wherein the fourth loop is formed of one of the first stent wire and the second stent wire and defines a fourth loop opening.

A fifteenth aspect is the stent device according to the fourteenth aspect, wherein the first loop, the second loop, the third loop, and the fourth loop form two double-loop structures.

A sixteenth aspect is the stent device according to the first aspect, wherein a number of the primary interlocking structure is equal to or less than the number of the secondary interlocking structure.

A seventeenth aspect is the stent device according to the first aspect, wherein the first stent wire does not include three consecutive loops along the alternating peaks and valleys.

An eighteenth aspect is the stent device according to the first aspect, wherein the first stent wire includes one loop among the four consecutive alternating peaks and valleys.

A nineteenth aspect is the stent device according to the first aspect, wherein the first stent wire includes two loops among the four consecutive alternating peaks and valleys.

A twelfth aspect is the stent device according to the first aspect, wherein the stent device comprises a stent delivery system including:
a sheath having a capability to carry the stent device; and
a pusher for pushing out the stent device from the sheath.

What is claimed is:

1. A stent formed by weaving wires, comprising:
a plurality of straight-line crossing portions, which are formed by crossing at least two straight-line portions of the wires and are arranged adjacent to each other in a circumferential direction of the stent; and
a plurality of interlocking portions configured by intersecting a peak-shaped bent portion, in which the wire is bent in a first direction side which is one side of a longitudinal axis direction of the stent and becomes convex, and a valley-shaped bent portion, in which the wire is bent in a second direction side which is the other side of the longitudinal axis direction and becomes convex, and arranged so as to be adjacent to each other in the circumferential direction of the stent,
wherein the interlocking portions and the straight-line crossing portions are arranged alternately in the longitudinal axis direction.

2. The stent according to claim 1, further comprising:
an upper interlocking portion and a lower interlocking portion, which are the interlocking portions; and
a first straight-line portion that is the straight-line portion continuous with the upper interlocking portion on the first direction side and continuous with the lower interlocking portion on the second direction side,
wherein the first straight-line portion constitutes the straight-line crossing portion with two or more other straight-line portions between the upper interlocking portion and the lower interlocking portion.

3. The stent according to claim 1, wherein
the plurality of straight-line crossing portions include
a first straight-line crossing portion where the first straight-line portion and the second straight-line portion intersect,
a second straight-line crossing portion where the first straight-line portion and the third straight-line portion intersect,
a third straight-line crossing portion where the third straight-line portion and the fourth straight-line portion intersect, and
a fourth straight-line crossing portion where the second straight-line portion and the fifth straight-line portion intersect,
the plurality of interlocking portions include
a first interlocking portion where a first peak, which is the peak-shaped bent portion, and a first valley, which is the valley-shaped bent portion, which are connected to the first direction side of the first straight-line portion intersect,
a second interlocking portion where a second peak, which is the peak-shaped bent portion, and a second valley, which is the valley-shaped bent portion, which are connected to the first direction side of the second straight-line portion and the fourth straight-line portion intersect and
a third interlocking portion where a third peak, which is the peak-shaped bent portion, and a third valley, which is the valley-shaped bent portion, which are connected to the first direction side of the third straight-line portion intersect,
a fourth interlocking portion where a fourth valley, which is the valley-shaped bent portion, and a fourth peak, which is the peak-shaped bent portion, which are connected to the second direction side of the first straight-line portion intersect,
a fifth interlocking portion where a fifth valley, which is the valley-shaped bent portion, and a fifth peak, which is the peak-shaped bent portion, which are connected to the second direction side of the third straight-line portion and the fifth straight-line portion intersect, and
a sixth interlocking portion where a sixth valley, which is the valley-shaped bend, and a sixth peak, which is the peak-shaped bend, which are connected to the second direction side of the second straight-line portion intersect.

4. The stent according to claim 1, wherein
the plurality of straight-line crossing portions include
a first straight-line crossing portion where the first straight-line portion and the second straight-line portion intersect,
a second straight-line crossing portion where the first straight-line portion and the third straight-line portion intersect,
a third straight-line crossing portion where the first straight-line portion and the fourth straight-line portion intersect,
a fourth straight-line crossing portion where the third straight-line portion and the fifth straight-line portion intersect, and
a fifth straight-line crossing portion where the third straight-line portion and the sixth straight-line portion intersect,
the plurality of interlocking portions include
a first interlocking portion where a first peak, which is the peak-shaped bent portion, and a first valley, which is the valley-shaped bent portion, which are connected to the first direction side of the first straight-line portion intersect,
a second interlocking portion where a second peak, which is the peak-shaped bent portion, and a second valley, which is the valley-shaped bent portion, which are connected to the first direction side of the second straight-line portion and the fifth straight-line portion intersect,
a third interlocking portion where a third peak, which is the peak-shaped bent portion, and a third valley, which is the valley-shaped bent portion, which are connected to the first direction side of the third straight-line portion intersect,
a fourth interlocking portion where a fourth valley, which is the valley-shaped bent portion, and a fourth peak, which is the peak-shaped bent portion, which are connected to the second direction side of the first straight-line portion intersect,
a fifth interlocking portion where a fifth valley, which is the valley-shaped bent portion, and a fifth peak, which is the peak-shaped bent portion, which are connected to the second direction side of the fourth straight-line portion and the sixth straight-line portion intersect, and
a sixth interlocking portion where a sixth valley, which is the valley-shaped bend, and a sixth peak, which is the peak-shaped bend, which are connected to the second direction side of the third straight-line portion intersect.

5. The stent according to claim 1, wherein
a first region, in which the plurality of straight-line crossing portions are spirally arranged along the longitudinal axis direction, and a second region, in which the plurality of interlocking portions are spirally arranged along the longitudinal axis direction, are alternately arranged in the longitudinal direction, and
the first regions arranged on both sides in the longitudinal direction across the second region are formed of different wires.

6. The stent according to claim 5, wherein the first regions arranged on both sides of the second region in the longitudinal direction have different numbers of overlapping wires.

7. The stent according to claim 1, wherein
the plurality of straight-line crossing portions have a central straight-line crossing portion where the first straight-line portion of the wire and the second straight-line portion of the wire intersect,
the plurality of interlocking portions include
a first interlocking portion where a first peak, which is the peak-shaped bent portion continuous with the first direction side of the first straight-line portion, and a first valley, which is the valley-shaped bent portion, intersect, and
a second interlocking portion where a second peak, which is the peak-shaped bent portion continuous with the first direction side of the second straight-line portion, and a second valley, which is the valley-shaped bent portion, intersect, and
the first interlocking portion and the second interlocking portion are arranged at different positions in the longitudinal axis direction.

8. The stent according to claim 7, wherein
the wire has a first wire and a second wire different from the first wire,
the first valley and the second peak are formed by the first wire, and
the first peak and the second valley are formed by the second wire.

9. The stent according to claim 7, wherein
the wire has a first wire and a second wire different from the first wire,
the first peak and the first valley are formed by the first wire, and
the second peak and the second valley are formed by the second wire.

10. The stent according to claim 7, wherein
the central straight-line crossing portion is arranged between the first interlocking portion and the second interlocking portion in the circumferential direction of the stent.

11. The stent according to claim 7, wherein
the first interlocking portion is arranged between the second interlocking portion and the central straight-line crossing portion in the longitudinal axis direction.

12. The stent according to claim 7, wherein
the plurality of interlocking portions further include
a third interlocking portion where a third valley, which is the valley-shaped bent portion continuous with the second direction side of the first straight-line portion, and a third peak, which is the peak-shaped bent portion, intersect, and
a fourth interlocking portion where a fourth valley, which is the valley-shaped bent portion continuous with the second direction side of the second straight-line portion, and a fourth peak, which is the peak-shaped bent portion, intersect, and
the third interlocking portion and the fourth interlocking portion are arranged at different positions in the longitudinal axis direction.

13. The stent according to claim 12, wherein the central straight-line crossing portion is arranged between the third interlocking portion and the fourth interlocking portion in the circumferential direction of the stent.

14. The stent according to claim 12, wherein the third interlocking portion is arranged between the central straight-line crossing portion and the fourth interlocking portion in the longitudinal axis direction.

15. The stent according to claim 1, wherein the first regions where the plurality of straight-line crossing portions are arranged and the second regions where the plurality of interlocking portions are arranged are arranged alternately in the longitudinal axis direction.

16. The stent according to claim 15, wherein the first region is spirally arranged along the longitudinal axis.

17. The stent according to claim 15, wherein the second region is spirally arranged along the longitudinal axis.

18. The stent according to claim 12, wherein
in the first interlocking portion, the first peak and the first valley intersect at the first crossing portion and the second crossing portion closer to the central straight-line crossing portion than the first crossing portion,
in the third interlocking portion, the third peak and the third valley intersect at the fourth crossing portion and the third crossing portion closer to the central straight-line crossing portion than the fourth crossing portion,
at the first crossing portion, the first peak passes outside the first valley,
at the second crossing portion, the first peak passes inside the first valley,
at the central straight-line crossing portion, the first straight-line portion passes outside the second straight-line portion,
at the third crossing portion, the third valley passes inside the third peak, and
at the fourth crossing portion, the third valley passes outside the third peak.

19. The stent according to claim 12, wherein
in the first interlocking portion, the first peak and the first valley intersect at the first crossing portion and the second crossing portion closer to the central straight-line crossing portion than the first crossing portion,
in the third interlocking portion, the third peak and the third valley intersect at the fourth crossing portion and the third crossing portion closer to the central straight-line crossing portion than the fourth crossing portion,
at the first crossing portion, the first peak passes inside the first valley,
at the second crossing portion, the first peak passes outside the first valley,
at the central straight-line crossing portion, the first straight-line passes outside the second straight line,
at the third crossing portion, the third valley passes outside the third peak, and
at the fourth crossing portion, the third valley passes inside the third peak.

20. The stent according to claim 12, wherein
in the first interlocking portion, the first peak and the first valley intersect at the first crossing portion and the second crossing portion closer to the central straight-line crossing portion than the first crossing portion,
in the third interlocking portion, the third peak and the third valley intersect at the fourth crossing portion and the third crossing portion closer to the central straight-line crossing portion than the fourth crossing portion,
at the first crossing portion, the first peak passes inside the first valley,
at the second crossing portion, the first peak passes outside the first valley,
at the central straight-line crossing portion, the first straight-line passes outside the second straight line,
at the third crossing portion, the third valley passes inside the third peak, and
at the fourth crossing portion, the third valley passes outside the third peak.

21. The stent according to claim 12, wherein
in the first interlocking portion, the first peak and the first valley intersect at the first crossing portion and the second crossing portion closer to the central straight-line crossing portion than the first crossing portion,
in the third interlocking portion, the third peak and the third valley intersect at the fourth crossing portion and the third crossing portion closer to the central straight-line crossing portion than the fourth crossing portion,
the central straight-line crossing portion includes
a first straight-line crossing portion in which
at the first crossing portion, the first peak passes outside the first valley,
at the second crossing portion, the first peak passes inside the first valley,
at the central straight-line crossing portion, the first straight-line passes outside the second straight line,
at the third crossing portion, the third valley passes inside the third peak, and
at the fourth crossing portion, the third valley passes outside the third peak,
a second straight-line crossing portion in which
at the first crossing portion, the first peak passes inside the first valley,
at the second crossing portion, the first peak passes outside the first valley,
at the central straight-line crossing portion, the first straight-line passes outside the second straight line,
at the third crossing portion, the third valley passes outside the third peak, and
at the fourth crossing portion, the third valley passes inside the third peak.
